(19) United States
(12) Patent Application Publication
(10) Pub. No.: US 2008/0051384 A1
(43) Pub. Date: Feb. 28, 2008

(54) ANTIVIRAL AGENTS

(75) Inventors: Franz Ulrich Schmitz, Mill Valley, CA (US); Christopher Don Roberts, Belmont, CA (US); Ronald Conrad Griffith, Escondido, CA (US); Janos Botyanszki, Fremont, CA (US); Son Minh Pham, San Francisco, CA (US)

Correspondence Address: FOLEY & LARDNER LLP, 975 PAGE MILL ROAD, PALO ALTO, CA, 94304, US

(73) Assignee: Genelabs Technologies, Inc.

(21) Appl. No.: 11/777,154

(22) Filed: Jul. 12, 2007

Related U.S. Application Data
(60) Provisional application No. 60/831,040, filed on Jul. 14, 2006.

(51) Int. Cl. A61K 31/397; A61K 31/407; A61K 31/4709; A61K 31/497; A61K 31/506; A61K 31/5377; A61P 31/14; C07D 205/02; C07D 215/00; C07D 239/02; C07D 279/12; C07D 401/02; C07D 401/14; C07D 403/02; C07D 403/14; C07D 413/02; C07D 413/14; C07D 495/04

(52) U.S. Cl. 514/210.18; 514/228.2; 514/233.8; 514/235.2; 514/254.08; 514/274; 514/311; 514/314; 514/338; 514/412; 544/128; 544/130; 544/316; 544/373; 544/58.2; 544/58.6; 546/152; 546/167; 546/276.7; 548/453; 548/953

(57) ABSTRACT

Disclosed are compounds and compositions of Formula (I) and their uses for treating Flaviviridae family virus infections.

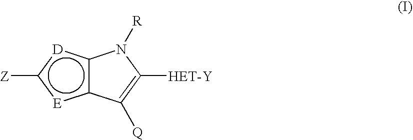

### CROSS-REFERENCE TO RELATED APPLICATION

This application claims priority to co-pending provisional application U.S. Ser. No. 60/831,040 filed on Jul. 14, 2006, which is incorporated herein by reference in its entirety.

### BACKGROUND OF THE INVENTION

1. Field of the Invention

The invention relates to the field of pharmaceutical chemistry, in particular to compounds, compositions and methods for treating viral infections in mammals mediated, at least in part, by a virus in the Flaviviridae family of viruses.

### REFERENCES

The following publications are cited in this application as superscript numbers
1. Szabo, E. et al., Pathol. Oncol. Res. 2003, 9:215-221.
2. Hoofnagle J. H., Hepatology 1997, 26:15 S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J. Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem. 39, 223-237, 2004.
9. Watashi, K. et al., Molecular Cell, 19, 111-122, 2005
10. Horsmans, Y. et al., Hepatology, 42, 724-731, 2005

2. State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al. show how antiviral activity can be achieved by inhibiting host cell cyclophilins.⁹ Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

The present invention is directed to novel compounds, compositions, and methods for treating of viral infections in mammals mediated, at least in part, by a member of the Flaviviridae family viruses such as HCV. Specifically, compounds of this invention are represented by Formula (I) or a pharmaceutically acceptable salt, ester, stereoisomer, prodrug, or tautomer thereof: 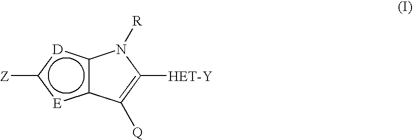
wherein:

Y is selected from the group consisting of aryl, heteroaryl, substituted aryl, and substituted heteroaryl;

HET is selected from the group consisting of a 6-membered arylene ring, a 6-membered heteroarylene ring containing 1, 2, or 3 heteroatoms selected from N, O, or S, and a bicyclic ring having the formula 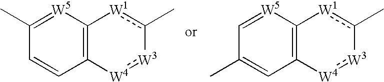
wherein HET is optionally substituted with (X)ₜ, X is selected from the group consisting of alkyl, substituted alkyl, alkoxy, substituted alkoxy, amino, substituted amino, halo, hydroxy, and nitro; t is an integer equal to 0, 1 or 2; W¹, W⁴, and W⁵ are independently N or CH; W³ is N, CH, or is a bond provided that no more than one nitrogen in the bicyclic ring is optionally oxidized to form an N-oxide; and each dashed line independently represents a single or double bond between the two adjoining atoms, provided that when one of dashed lines is a single bond, the adjoining atoms are each substituted with 1 or 2 hydrogen atoms to satisfy its valency;

one of D or E is C—R^{a} and the other of D or E is S;

R^{a} and R are independently selected from the group consisting of hydrogen, alkyl, and substituted alkyl;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²² and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

### DETAILED DESCRIPTION OF THE INVENTION

### Definitions

It is to be understood that the terminology used herein is for the purpose of describing particular embodiments only and is not intended to limit the scope of the present invention. In this specification and in the claims that follow, reference will be made to a number of terms that shall be defined to have the following meanings:

As used herein, “alkyl” refers to monovalent alkyl groups having from 1 to 10 carbon atoms, preferably from 1 to 5 carbon atoms and more preferably 1 to 3 carbon atoms. This term is exemplified by groups such as methyl, ethyl, n-propyl, iso-propyl, n-butyl, t-butyl, n-pentyl and the like.

“Substituted alkyl” refers to an alkyl group having from 1 to 3, and preferably 1 to 2, substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, cycloalkenyl, substituted cycloalkenyl, heteroaryl, substituted heteroaryl, heterocyclic, and substituted heterocyclic.

“Alkoxy” refers to the group “alkyl-O—” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O—”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)— cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)—.

“Acylamino” refers to the group —C(O)NR^{f}R^{g} where R^{f} and R^{g} is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{f} and R^{g} are joined to form together with the nitrogen atom a heterocyclic or substituted heterocyclic ring.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—.

“Alkenyl” refers to alkenyl group having from 2 to 10 carbon atoms, preferably having from 2 to 6 carbon atoms, and more preferably 2 to 4 carbon atoms and having at least 1 and preferably from 1-2 sites of alkenyl unsaturation.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to a vinyl carbon atom.

“Alkynyl” refers to alkynyl group having from 2 to 10 carbon atoms, preferably having from 2 to 6 carbon atoms, and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy ester, cycloalkyl, substituted cycloalkyl, heteroaryl, substituted heteroaryl, heterocyclic, and substituted heterocyclic provided that any hydroxyl substitution is not pendent to an acetylenic carbon atom.

“Amino” refers to the group —NH₂.

“Substituted amino” refers to the group —NR^{h}Rⁱ where R^{h} and Rⁱ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{h} and Rⁱ are joined, together with the nitrogen bound thereto to form a heterocyclic or substituted heterocyclic group provided that R^{h} and Rⁱ are both not hydrogen. When R^{h} is hydrogen and Rⁱ is alkyl, the substituted amino group is sometimes referred to herein as alkylamino. When R^{h} and Rⁱ are alkyl, the substituted amino group is sometimes referred to herein as dialkylamino.

“Aminoacyl” refers to the groups —NR^{j}C(O)alkyl, —NR^{j}C(O)substituted alkyl, —NR^{j}C(O)-cycloalkyl, —NR^{j}C(O)substituted cycloalkyl, —NR^{j}C(O)alkenyl, —NR^{j}C(O)substituted alkenyl, —NR^{j}C(O)alkynyl, —NR^{j}C(O)substituted alkynyl, —NR^{j}C(O)aryl, —NR^{j}C(O)substituted aryl, —NRⁱC(O)heteroaryl, —NR^{j}C(O)substituted heteroaryl, —NR^{j}C(O)heterocyclic, and —NR^{j}C(O)substituted heterocyclic where R^{j} is hydrogen or alkyl.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is to an aromatic ring atom. Preferred aryls include phenyl and naphthyl.

“Aralkyl” or “arylalkyl” refers to the group aryl-alkyl- and includes, for example, benzyl.

“Substituted aryl” refers to aryl groups which are substituted with from 1 to 3 substituents, and preferably 1 to 2 substituents, selected from the group consisting of hydroxy, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxy, carboxy esters, cyano, thiol, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Arylene” and “substituted arylene” refer to divalent aryl and substituted aryl groups as defined above. “Phenylene” is a 6-membered optionally substituted arylene group and includes, for example, 1,2-phenylene, 1,3-phenylene, and 1,4-phenylene.

“Aryloxy” refers to the group aryl-O— that includes, by way of example, phenoxy, naphthoxy, and the like.

“Substituted aryloxy” refers to substituted aryl-O— groups.

“Carboxy” refers to —C(═O)OH or salts thereof.

“Carboxy esters” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic. Preferred carboxy esters are —C(O)O-alkyl, —C(O)O-substituted alkyl, —C(O)O-aryl, and —C(O)O-substituted aryl.

“Cycloalkyl” refers to cyclic alkyl groups of from 3 to 10 carbon atoms having single or multiple cyclic rings optionally comprising 1 to 3 exo carbonyl or thiocarbonyl groups. Suitable cycloalkyl groups include, by way of example, adamantyl, cyclopropyl, cyclobutyl, cyclopentyl, cyclohexyl, cyclooctyl, 3-oxocyclohexyl, and the like. In multiple condensed rings, one or more of the rings may be other than cycloalkyl (e.g., aryl, heteroaryl or heterocyclic) provided that the point of attachment is to a carbon ring atom of the cycloalkyl group. In one embodiment, the cycloalkyl group does not comprise 1 to 3 exo carbonyl or thiocarbonyl groups. In another embodiment, the cycloalkyl group does comprise 1 to 3 exo carbonyl or thiocarbonyl groups. It is understood, that the term “exo” refers to the attachment of a carbonyl or thiocarbonyl to a carbon ring atom of the cycloalkyl group.

“Substituted cycloalkyl” refers to a cycloalkyl group, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic.

“Cycloalkenyl” refers to cyclic alkenyl but not aromatic groups of from 5 to 10 carbon atoms having single or multiple cyclic rings optionally comprising 1 to 3 exo carbonyl or thiocarbonyl groups. Suitable cycloalkenyl groups include, by way of example, cyclopentyl, cyclohexenyl, cyclooctenyl, 3-oxocyclohexenyl, and the like. In multiple condensed rings, one or more of the rings may be other than cycloalkenyl (e.g., aryl, heteroaryl or heterocyclic) provided that the point of attachment is to a carbon ring atom of the cycloalkyl group. In one embodiment, the cycloalkenyl group does not comprise 1 to 3 exo carbonyl or thiocarbonyl groups. In another embodiment, the cycloalkenyl group does comprise 1 to 3 exo carbonyl or thiocarbonyl groups. It is understood, that the term “exo” refers to the attachment of a carbonyl or thiocarbonyl to a carbon ring atom of the cycloalkenyl group.

“Substituted cycloalkenyl” refers to cycloalkenyl groups having from 1 to 5 substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic provided that for hydroxyl substituents the point of attachment is not to a vinyl carbon atom.

“Cycloalkoxy” refers to —O-cycloalkyl groups.

“Substituted cycloalkoxy” refers to —O-substituted cycloalkyl groups.

The term “guanidino” refers to the group —NHC(═NH)NH₂ and the term “substituted guanidino” refers to —NR^{p}C(═NR^{p})N(R^{p})₂ where each R^{p} is independently hydrogen or alkyl.

“Halo” or “halogen” refers to fluoro, chloro, bromo and iodo and preferably is fluoro or chloro.

“Haloalkyl” refers to an alkyl group substituted with 1 to 5 halogen groups. An example of haloalkyl is CF₃.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur, within the ring. Preferably, such heteroaryl groups are aromatic groups of from 1 to 15 carbon atoms, preferably from 1 to 10 carbon atoms, and 1 to 4 heteroatoms selected from the group consisting of oxygen, nitrogen, and sulfur within the ring. Such heteroaryl groups can have a single ring (e.g., pyridyl or furyl) or multiple condensed rings (e.g., indolizinyl or benzothienyl). The sulfur atom(s) in the heteroaryl group may optionally be oxidized to sulfoxide and sulfone moieties.

“Substituted heteroaryl” refers to heteroaryl groups that are substituted with from 1 to 3 substituents selected from the same group of substituents defined for substituted aryl.

When a specific heteroaryl is defined as “substituted”, e.g., substituted quinoline, it is understood that such a heteroaryl contains the 1 to 3 substituents as recited above.

“Heteroarylene” and “substituted heteroarylene” refer to divalent heteroaryl and substituted heteroaryl groups as defined above.

“Heteroaryloxy” refers to the group —O-heteroaryl and “substituted heteroaryloxy” refers to the group —O-substituted heteroaryl.

“Heterocycle” or “heterocyclic” or “heterocyclyl” refers to a saturated or unsaturated group having a single ring or multiple condensed rings, from 1 to 10 carbon atoms and from 1 to 4 hetero atoms selected from the group consisting of nitrogen, sulfur or oxygen within the ring which ring may optionally comprise 1 to 3 exo carbonyl or thiocarbonyl groups. Preferably, such heterocyclic groups are saturated or unsaturated group having a single ring or multiple condensed rings, from 1 to 10 carbon atoms and from 1 to 4 hetero atoms selected from the group consisting of nitrogen, sulfur, or oxygen within the ring. The sulfur atom(s) in the heteroaryl group may optionally be oxidized to sulfoxide and sulfone moieties.

In multiple condensed rings, one or more of the rings may be other than heterocyclic (e.g., aryl, heteroaryl or cycloalkyl) provided that the point of attachment is to a heterocyclic ring atom. In one embodiment, the heterocyclic group does not comprise 1 to 3 exo carbonyl or thiocarbonyl groups. In a preferred embodiment, the heterocyclic group does comprise 1 to 3 exo carbonyl or thiocarbonyl groups. It is understood, that the term “exo” refers to the attachment of a carbonyl or thiocarbonyl to a carbon ring atom of the heterocyclic group.

“Substituted heterocyclic” refers to heterocycle groups that are substituted with from 1 to 3 of the same substituents as defined for substituted cycloalkyl. Preferred substituents for substituted heterocyclic groups include heterocyclic groups having from 1 to 5 having substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxy, carboxy esters, cycloalkyl, substituted cycloalkyl, heteroaryl, substituted heteroaryl, heterocyclic, and substituted heterocyclic.

When a specific heterocyclic is defined as “substituted”, e.g., substituted morpholino, it is understood that such a heterocycle contains the 1 to 3 substituents as recited above.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

The term “thiol” refers to the group —SH.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR^{k}, —PO₂(R^{k})₂, —CN, —PO₃(R^{k})₂, —OR^{k}, —SR^{k}, —NHCOR^{k}, —N(R^{k})₂, —CON(R^{k})₂, —CONH(O)R^{k}, —CONHNHSO₂R^{k}, —COHNSO₂R^{k}, and —CONR^{k}CN, where R^{k} is selected from hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, thio, thioalkyl, alkylthio, sulfonyl, alkyl, alkenyl or alkynyl, aryl, aralkyl, cycloalkyl, heteroaryl, heterocycle, and CO₂R^{m} where R^{m} is hydrogen alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred isosteres contemplated by this invention: 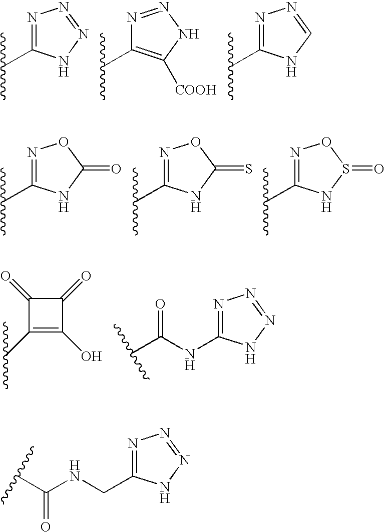
where the atoms of said ring structure may be optionally substituted at one or more positions with R^{k}. The present invention contemplates that when chemical substituents are added to a carboxylic isostere then the inventive compound retains the properties of a carboxylic isostere. The present invention contemplates that when a carboxylic isostere is optionally substituted with one or more moieties selected from R^{k}, then the substitution cannot eliminate the carboxylic acid isosteric properties of the inventive compound. The present invention contemplates that the placement of one or more R^{k} substituents upon the carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

“Carboxylic acid bioisosteres” are compounds that behave as isosteres of carboxylic acids under biological conditions.

Other carboxylic acid isosteres not specifically exemplified or described in this specification are also contemplated by the present invention

“Metabolite” refers to any derivative produced in a subject after administration of a parent compound. The metabolite may be produced from the parent compound by various biochemical transformations in the subject such as, for example, oxidation, reduction, hydrolysis, or conjugation. Metabolites include, for example, oxides and demethylated derivatives.

“Thiocarbonyl” refers to the group C(═S).

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Prodrug” refers to art recognized modifications to one or more functional groups which functional groups are metabolized in vivo to provide a compound of this invention or an active metabolite thereof. Such functional groups are well known in the art including acyl groups for hydroxyl and/or amino substitution, esters of mono-, di- and tri-phosphates wherein one or more of the pendent hydroxyl groups have been converted to an alkoxy, a substituted alkoxy, an aryloxy or a substituted aryloxy group, and the like.

“Treating” or “treatment” of a disease in a refers to 1) preventing the disease from occurring in a patient that is predisposed or does not yet display symptoms of the disease; 2) inhibiting the disease or arresting its development; or 3) ameliorating or causing regression of the disease. “Patient” refers to mammals and includes humans and non-human mammals.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moeity such as pyrazoles, imidazoles, benzimidazoles, triazoles, and tetrazoles.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—; the term “alkylaryloxy” refers to the group alkyl-aryl-O—; the term “arylalkyloxy” refers to the group aryl-alkyl-O—, “thioalkyl” refers to SH-alkyl-; “alkylthio” refers to alkyl-S— etc. Various substituents may also have alternate but equivalent names. For example, the term 2-oxo-ethyl and the term carbonylmethyl both refer to the —C(O)CH₂— group.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups or a hydroxy group alpha to ethenylic or acetylenic unsaturation). Such impermissible substitution patterns are well known to the skilled artisan.

Accordingly, provided are compounds of Formula (I) or a pharmaceutically acceptable salt, ester, stereoisomer, prodrug, or tautomer thereof: 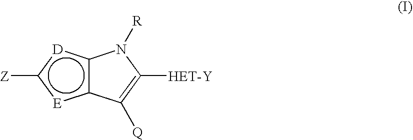
wherein:

Y is selected from the group consisting of aryl, heteroaryl, substituted aryl, and substituted heteroaryl;

HET is selected from the group consisting of a 6-membered arylene ring, a 6-membered heteroarylene ring containing 1, 2, or 3 heteroatoms selected from N, O, or S, and a bicyclic ring having the formula 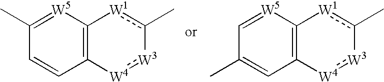
wherein HET is optionally substituted with (X)ₜ, X is selected from the group consisting of alkyl, substituted alkyl, alkoxy, substituted alkoxy, amino, substituted amino, halo, hydroxy, and nitro; t is an integer equal to 0, 1 or 2; W¹, W⁴, and W⁵ are independently N or CH; W³ is N, CH, or is a bond provided that no more than one nitrogen in the bicyclic ring is optionally oxidized to form an N-oxide; and each dashed line independently represents a single or double bond between the two adjoining atoms, provided that when one of dashed lines is a single bond, the adjoining atoms are each substituted with 1 or 2 hydrogen atoms to satisfy its valency;

one of D or E is C—R^{a} and the other of D or E is S;

R^{a} and R are independently selected from the group consisting of hydrogen, alkyl, and substituted alkyl;

Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and

Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²² and R²³ are independently hydrogen, alkyl, substituted alkyl, cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl, R¹ is selected from —OR⁷ and —NR⁸R⁹ where R⁷ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
   R² and R^{2′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
   or, alternatively, R² and R^{2′} as defined are taken together with the carbon atom pendent thereto to form a cycloalkyl, substituted cycloalkyl, heterocyclic or substituted heterocyclic group,
   or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
   R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

In another embodiment, provided is a compound having Formula (Ia) or a pharmaceutically acceptable salt or tautomer thereof: 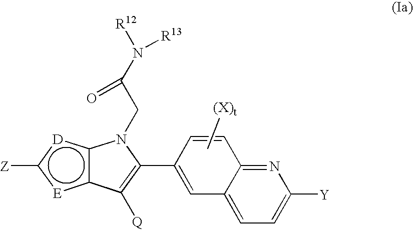
wherein:

Y is selected from the group consisting of substituted aryl and substituted heteroaryl;

X is independently selected from the group consisting of amino, nitro, alkyl, haloalkyl, and halo;

t is an integer equal to 0, 1 or 2;

Q is selected from the group consisting of cyclohexyl and cyclopentyl;

R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms;

one of A or B is C—R^{a} and the other of A or B is S;

R^{a} is selected from the group consisting of hydrogen, alkyl, and substituted alkyl; and

Z is selected from the group consisting of carboxy, carboxy ester, and a carboxylic acid isostere.

In other embodiments, the present invention provides compounds of Formulae (Ib)-(Is): 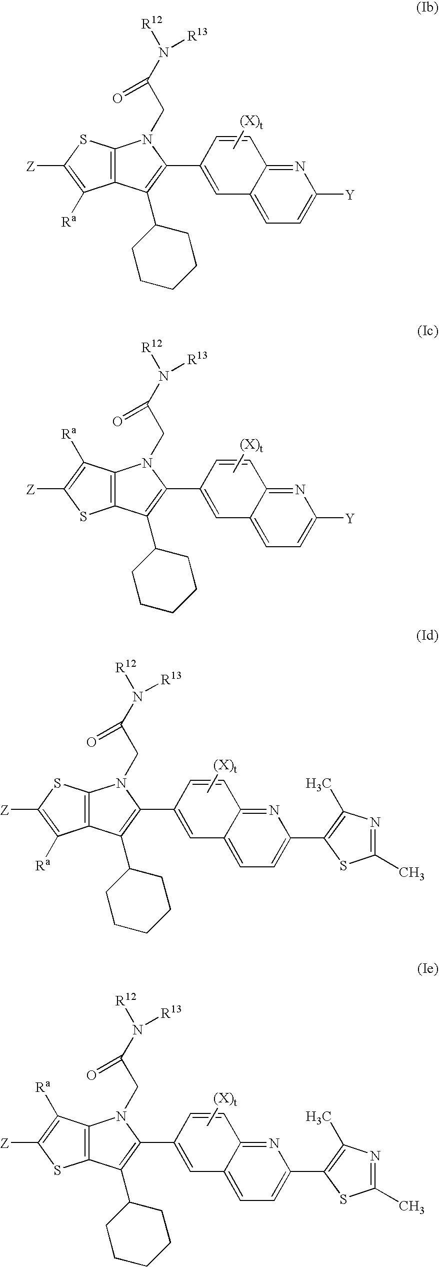 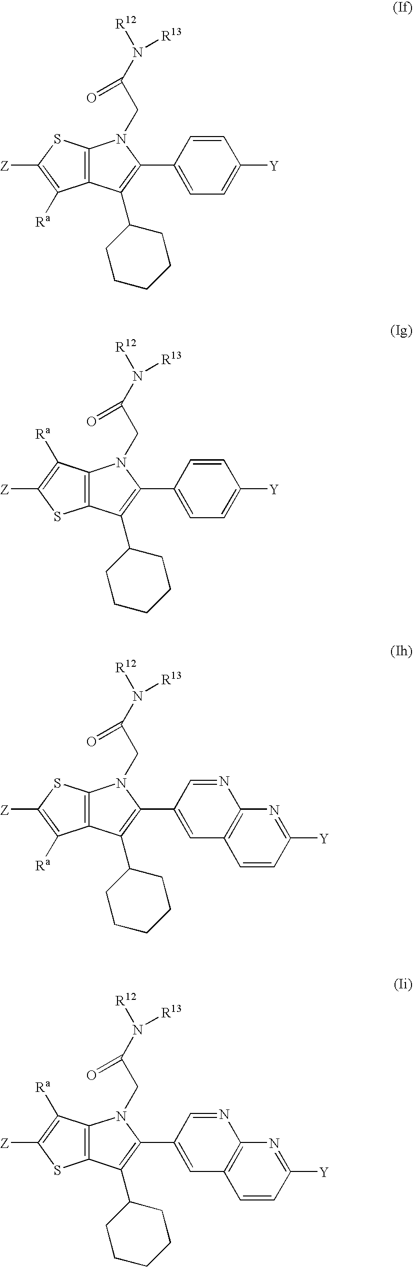 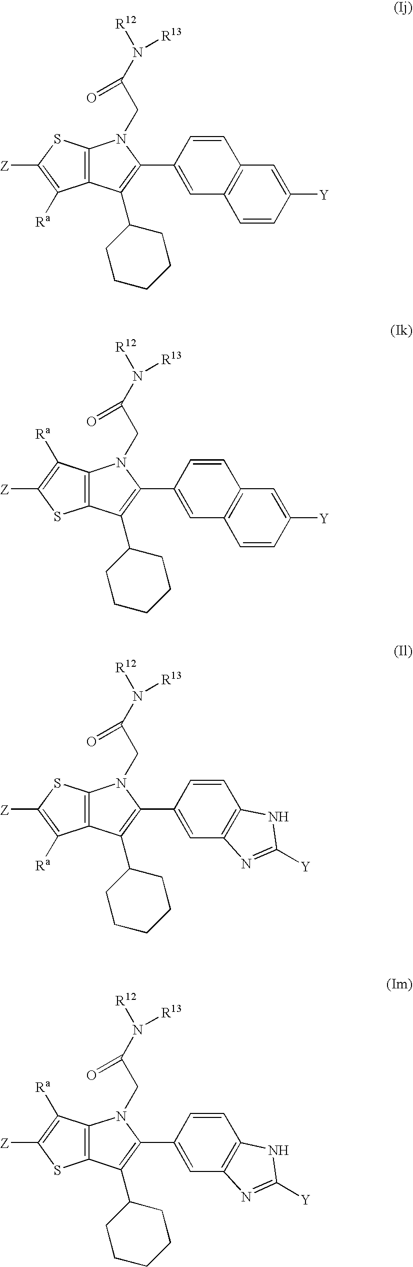 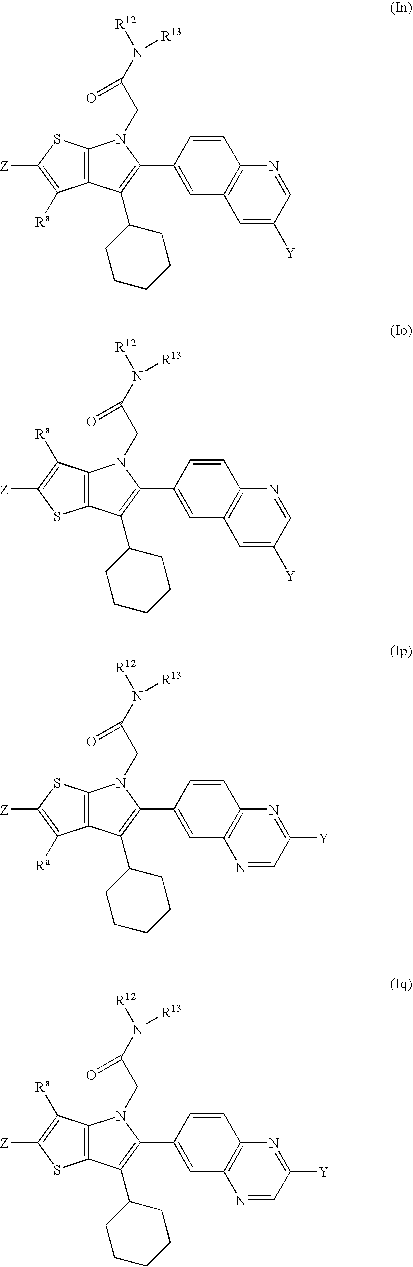 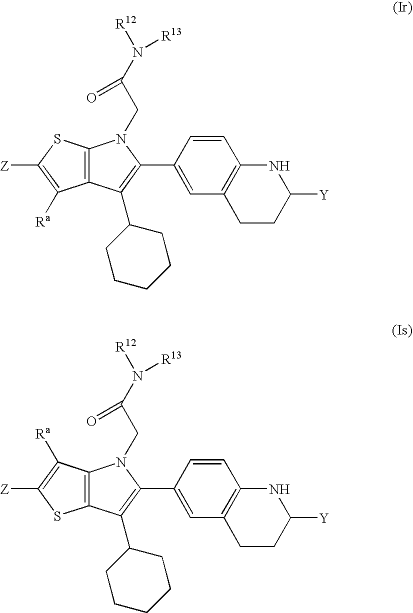
wherein Z, R^{a}, and Y are as previously defined in Formula (I) and R¹² and R¹³ are as previously defined for Formula (Ia).

In some embodiments of each of Formula (I) and (Ia) E is S. In other embodiments, D is CH and E is S.

In some embodiments of each of Formula (I) and (Ia)-(Is) where appropriate, R^{a} is hydrogen. In other embodiments, R^{a} is substituted alkyl, substituted amino, or substituted aminoalkyl. In some aspects, R^{a} is selected from the following substituents:

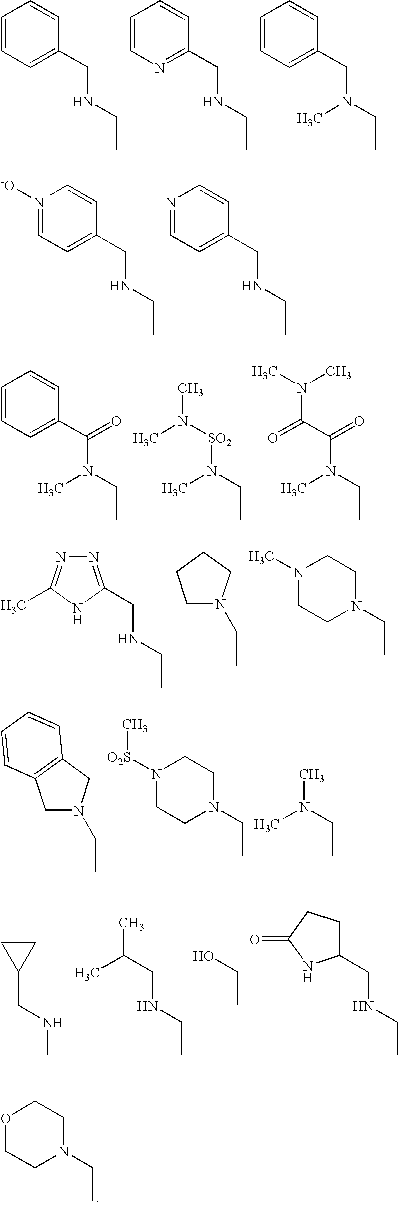

In some embodiments of each of Formula (I) and (Ia)-(Is) where appropriate, Q is cycloalkyl or substituted cycloalkyl. In some embodiments Q is cycloalkyl. In other embodiments, Q is cycloalkenyl. In another embodiment Q is cyclohexyl. In another embodiment Q is cyclohexenyl. In yet another embodiment T is cyclopentyl.

In some embodiments of each of Formula (I) and (Ia)-(Is) where appropriate, Z is carboxy or carboxy ester. In another embodiment Z is selected from —C(═O)OH, and —C(═O)OR″ where R″ is alkyl. In another embodiment Z is selected from carboxy, methyl carboxylate, and ethyl carboxylate. In yet another embodiment Z is —C(═O)OH.

In another embodiment Z is a carboxylic acid isostere. In another embodiment the carboxylic acid isostere is a carboxylic acid bioisostere. In another embodiment the carboxylic acid isostere is selected from 1H-tetrazol-5-yl and 5-oxo-4,5-dihydro-1,2,4-oxadiazol-3-yl.

In another embodiment Z is —C(═O)NR⁸R⁹ where R⁸ is hydrogen and R⁹ is selected from the group consisting of alkyl, substituted alkyl, aryl, substituted aryl, heteroaryl, substituted heteroaryl, heterocyclic and substituted heterocyclic. In another embodiment where Z is —C(═O)NR⁸R⁹ and R⁸ is hydrogen, R⁹ is substituted alkyl. In another embodiment where Z is —C(═O)NR⁸R⁹ and R⁸ is hydrogen, and R⁹ is substituted alkyl, the substituted alkyl comprises 1 to 2 substituents selected from the group consisting of sulfonic acid (SO₃H), carboxy, carboxy ester, amino, substituted amino, aryl, substituted aryl, heteroaryl and substituted heteroaryl. In another embodiment where Z is —C(═O)NR⁸R⁹ and R⁸ is hydrogen, and R⁹ is substituted alkyl, the substituted alkyl group is selected from the group consisting of 3,4-dimethoxybenzyl, 3,4-dihydroxybenzyl, 3-methoxy-4-hydroxybenzyl, 4-aminosulfonylbenzyl, 4-methylsulfonylbenzyl, (1-methyl-piperidin-3-yl)methyl, (1-methyl-pyrrolidin-3-yl)methyl, fur-2-ylmethyl, 6-methylpyridin-2-ylmethyl, 2-(1-methyl-pyrrolidin-3-yl)ethyl, 1-phenylethyl, 1-(3-methoxyphenyl)-ethyl, 1-(4-methoxyphenyl)-ethyl, N′,N′-dimethylaminoethyl, and 2-(1H-pyrazol-1-yl)ethyl.

In another embodiment Z is selected from N-methyl carboxamide, N,N-dimethylcarboxamido, N-isopropyl-carboxamido, N-allyl-carboxamido, and 5-hydroxy-tryptophan-carbonyl.

In another embodiment Z is —C(═O)NR⁸R⁹ wherein R⁹ is aryl or substituted aryl. In another embodiment where Z is —C(═O)NR⁸R⁹, R⁹ is substituted aryl. In another embodiment where Z is —C(═O)NR⁸R⁹, R⁹ is selected from the group consisting of 7-hydroxynaphth-1-yl, 6-hydroxynaphth-1-yl, 5-hydroxynaphth-1-yl, 6-carboxynaphth-2-yl, (4-HOOCCH₂-)phenyl, (3,4-dicarboxy)phenyl, 3-carboxyphenyl, 3-carboxy-4-hydroxyphenyl and 2-biphenyl.

In another embodiment Z is —C(═O)NR⁸R⁹ where R⁹ is heteroaryl or substituted heteroaryl. In another embodiment where Z is —C(═O)NR⁸R⁹, R⁹ is substituted heteroaryl. In another embodiment where Z is —C(═O)NR⁸R⁹ and R⁹ is substituted heteroaryl, the substituted heteroaryl is selected from the group consisting of 4-methyl-2-oxo-2H-chromen-7-yl, 1-phenyl-4-carboxy-1H-pyrazol-5-yl, 5-carboxypyrid-2-yl, 2-carboxypyrazin-3-yl, and 3-carboxythien-2-yl.

In another embodiment Z is —C(═O)NR⁸R⁹ where R⁹ is heterocyclic. In another embodiment where Z is —C(═O)NR⁸R⁹ and R⁹ is heterocyclic, the heterocyclic group is N-morpholino, tetrahydrofuranyl, and 1,1-dioxidotetrahydrothienyl.

In another embodiment Z is —C(═O)NR⁸R⁹ where R⁸ and R⁹, together with the nitrogen atom pendent thereto, form a heterocyclic or substituted heterocyclic ring. In another embodiment where Z is —C(═O)NR⁸R⁹ and R⁸ and R⁹, together with the nitrogen atom pendent thereto form a ring, the heterocyclic and substituted heterocyclic rings comprise 4 to 8 membered rings containing 1 to 3 heteroatoms. In another embodiment where Z is —C(═O)NR⁸R⁹ and R⁸ and R⁹, together with the nitrogen atom pendent thereto form an optionally substituted heterocyclic ring, the 1 to 3 heteroatoms comprises 1 to 2 nitrogen atoms. In another embodiment where Z is —C(═O)NR⁸R⁹ and R⁸ and R⁹, together with the nitrogen atom pendent thereto form an optionally substituted heterocyclic ring, the heterocyclic or substituted heterocyclic ring is selected from the group consisting of piperidine, substituted piperidine, piperazine, substituted piperazine, morpholino, substituted morpholino, thiomorpholino and substituted thiomorpholino wherein the sulfur atom of the thiomorpholino or substituted thiomorpholino ring is optionally oxidized to provide for sulfoxide and sulfone moieties. In another embodiment where Z is —C(═O)NR⁸R⁹ and R⁸ and R⁹, together with the nitrogen atom pendent thereto form an optionally substituted heterocyclic ring, the heterocyclic or substituted heterocyclic ring is selected from the group consisting of 4-hydroxypiperidin-1-yl, 1,2,3,4-tetrahydro-3-carboxy-isoquinolin-2-yl, 4-methylpiperizin-1-yl, morpholin-4-yl, thiomorpholin-4-yl, 4-methyl-piperazin-1-yl, and 2-oxo-piperazinyl.

In another embodiment, Z is —C(X)N(R³)CR²R^{2′}C(═O)R¹.

In another embodiment, Z is —C(O)NHCHR²C(═O)R¹.

In another embodiment when Z is —C(X)N(R³)CR²R^{2′}C(═O)R¹ or —C(O)NHCHR²C(═O)R¹, R² is selected from the group consisting of hydrogen, alkyl, substituted alkyl, cycloalkyl, substituted cycloalkyl, aryl, substituted aryl, heteroaryl and substituted heteroaryl. In another embodiment where Z is —C(X)N(R³)CR²R^{2′}C(═O)R¹ or —C(O)NHCHR²C(═O)R¹, R² is selected from the group consisting of hydrogen, alkyl, substituted alkyl, cycloalkyl, and substituted cycloalkyl. In another embodiment where Z is —C(X)N(R³)CR²R^{2′}C(═O)R¹ or —C(O)NHCHR²C(═O)R¹, R² is selected from the group consisting of hydrogen, methyl, 1-methylprop-1-yl, sec-butyl, hydroxymethyl, 1-hydroxyeth-1-yl, 4-amino-n-butyl, 2-carboxyeth-1-yl, carboxymethyl, benzyl, (1H-imidazol-4-yl)methyl, (4-phenyl)benzyl, (4-phenylcarbonyl)benzyl, cyclohexylmethyl, cyclohexyl, 2-methylthioeth-1-yl, iso-propyl, carbamoylmethyl, 2-carbamoyleth-1-yl, (4-hydroxy)benzyl, and 3-guanindino-n-propyl.

In another embodiment when Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino. In another embodiment where Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino, and R² and R³, together with the carbon atom and nitrogen atom bound thereto respectively, are joined to form a heterocyclic or substituted heterocyclic group. In another embodiment where Z is —C(X)N(R³)CR²R^{2′}C(═O)R¹ or —C(O)NHCHR²C(═O)R¹, R¹ is selected from the group consisting of hydroxy, alkoxy, amino(N-morpholino), amino, and substituted amino and R² and R³, together with the carbon atom and nitrogen atom bound thereto respectively, are joined to form a heterocyclic or substituted heterocyclic group, the heterocyclic and substituted heterocyclic groups are selected from the group consisting of pyrrolidinyl, 2-carboxy-pyrrolidinyl, 2-carboxy-4-hydroxypyrrolidinyl, and 3-carboxy-1,2,3,4-tetrahydroisoquinolin-3-yl.

In another embodiment, Z is selected from 1-carboxamidocyclopent-1-ylaminocarbonyl, 1-carboxamido-1-methyl-eth-1-ylaminocarbonyl, 5-carboxy-1,3-dioxan-5-ylaminocarbonyl, 1-(N-methylcarboxamido)-1-(methyl)-eth-1-ylaminocarbonyl, 1-(N,N-dimethylcarboxamido)-1-(methyl) -eth-1-ylaminocarbonyl, 1-carboxy-1-methyl-eth-1-ylaminocarbonyl, 1-(N-methylcarboxamido)-cyclobutanaminocarbonyl, 1-carboxamido-cyclobutanaminocarbonyl, 1-(N,N-dimethylcarboxamido)-cyclobutanaminocarbonyl, 1-(N-methylcarboxamido)-cyclopentanaminocarbonyl, 1-(N,N-dimethylcarboxamido)-cyclopentanaminocarbonyl, 1-(carboxamido)-cyclopentanaminocarbonyl, 3-[N-(4-(2-aminothiazol-4-yl)phenyl)aminocarbonyl]-piperidin-3-ylaminocarbonyl, 3-carboxamido-pyrrolidin-3-ylaminocarbonyl, [1-(4-(acrylic acid)-phenyl)aminocarbonyl) -cyclobutan-1-yl]aminocarbonyl, and [1-methyl-1-(4-(acrylic acid)-phenyl)aminocarbonyl) -eth-1-yl]aminocarbonyl.

In another embodiment, Z is —C(O)NR²¹S(O)₂R⁴. In another embodiment where Z is —C(O)NR²¹S(O)₂R⁴, R⁴ is selected from the group consisting of alkyl, substituted alkyl, aryl, substituted aryl, heteroaryl and substituted heteroaryl. In another embodiment where Z is —C(O)NR²¹S(O)₂R⁴, R⁴ is methyl, ethyl, isopropyl, propyl, trifluoromethyl, 2,2,2-trifluoroethyl, phenyl, benzyl, phenethyl, 4-bromophenyl, 4-nitrophenyl or 4-methylphenyl, 4-methoxyphenyl, 2-aminoethyl, 2-(dimethylamino)ethyl, 2-N-benzyloxyaminoethyl, pyridinyl, thienyl, 2-chlorothien-5-yl, 2-methoxycarbonylphenyl, naphthyl, 3-chlorophenyl, 2-bromophenyl, 2-chlorophenyl, 4-trifluoromethoxyphenyl, 2,5-difluorophenyl, 4-fluorophenyl, 2-methylphenyl, 6-ethoxybenzo[d]thiazo-2-yl, 4-chlorophenyl, 3-methyl-5-fluorobenzo[b]thiophen-1-yl, 4-acetylaminophenyl, quinolin-8-yl, 4-t-butylphenyl, cyclopropyl, 2,5-dimethoxyphenyl, 2,5-dichloro-4-bromo-thien-3-yl, 2,5-dichloro-thien-3-yl, 2,6-dichlorophenyl, 1,3-dimethyl-5-chloro-1H-pyrazol-4-yl, 3,5-dimethylisoxazol-4-yl, benzo[c][1,2,5]thiadiazol-4-yl, 2,6-difluorophenyl, 6-chloro-imidazo[2,1-b]thiazol-5-yl, 2-(methylsulfonyl)phenyl, isoquinolin-8-yl, 2-methoxy-4-methylphenyl, 1,3,5-trimethyl-1H-pyrazol-4-yl, 1-phenyl-5-methyl-1H-pyrazol-4-yl, 2,4,6-trimethylphenyl, and 2-carbamoyl-eth-1-yl.

In another embodiment, Z is selected from hydrogen, halo, alkyl, alkoxy, amino, substituted amino, and cyano.

In another embodiment, Z is —C(X²)—N(R³)CR²⁵R²⁶R²⁷, wherein X² and R³ are defined above, and R²⁵, R²⁶ and R²⁷ are alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group.

In another embodiment, Z is selected from 1-(6-(3-carboxyprop-2-en-1-yl)-1H-benzo[d]imidazol-2-yl)cyclobutanaminocarbonyl, 3-(6-(3-carboxyprop-2-en-1-yl)-1H-benzo[d]imidazol-2-yl)-1-methylpyrrolidin-3-aminocarbonyl, 1-(1-methyl-6-(3-carboxyprop-2-en-1-yl)-1H-benzo[d]imidazol-2-yl)cyclobutanaminocarbonyl, 1-(benzofuran-2-yl)-5-carboxy-cyclobutanaminocarbonyl, 1-(2-methylthiazol-4-yl)-cyclobutanaminocarbonyl, 1-(2-acetylamino-thiazol-4-yl)-cyclobutanamino, 1-(2-methylamino-thiazol-4-yl)-cyclobutanaminocarbonyl, 1-(2-ethylthiazol-4-yl)-cyclobutanaminocarbonyl, and 1-(cyano)-cyclobutanaminocarbonyl.

In still other embodiments of each of Formula (I) and (Ia)-(Is) where appropriate, Z is carboxy, carboxy ester, carboxylic acid isostere, —C(O)NR⁸R⁹, or —C(O)NHS(O)₂R⁴, wherein R⁸ and R⁹ are as defined above and R⁴ is alkyl or aryl. In other embodiments Z is carboxy, methyl carboxylate, ethyl carboxylate, 6-(β-D-glucuronic acid) ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl. In still other embodiments Z is carboxy. In yet other embodiments Z is —C(═O)OH.

In some embodiments of each of Formula (I) and (Ia)-(Is) where appropriate, Z¹ is selected from the group consisting of hydrogen, halo, alkyl, and haloalkyl.

In some embodiments of each of Formula (I) and (Ia)-(Is) where appropriate, R is CᵥH₂ᵥ—C(O)—OR²³ where v is 1, 2 or 3; and R²³ is hydrogen, alkyl or substituted alkyl. In another embodiment where R is CᵥH₂ᵥ—C(O)—OR²³, v is 1. In another embodiment where R is CᵥH₂ᵥ—C(O)—OR²³, R is carboxymethyl or methylcarboxymethyl.

In another embodiment R is hydrogen.

In another embodiment R is CᵥH₂ᵥ—C(O)—NR¹²R¹³ where v is 1, 2 or 3; R¹² and R¹³ are selected from hydrogen, alkyl, substituted alkyl, alkenyl, substituted alkenyl, alkynyl, substituted alkynyl alkoxy, substituted alkoxy and —(CH₂)₀₋₃R¹⁶; and R¹⁶ is aryl, heteroaryl, heterocyclic, —NR¹⁷R¹⁸; and R¹⁷ and R¹⁸ are independently selected from hydrogen, and alkyl, or alternatively, R¹⁷ and R¹⁸ together with the nitrogen atom to which they are attached join to form a heterocyclic ring with 4 to 7 ring atoms; or, alternatively, R¹² and R¹³ and the nitrogen atom to which they are attached form a heterocyclic or substituted heterocyclic ring; provided that both R¹² and R¹³ are not alkoxy and/or substituted alkoxy. In another embodiment v is 1. In another embodiment where R is CᵥH₂ᵥ—C(O)—NR¹²R¹³, the NR¹²R¹³ group is selected from N,N-dimethylamino-carbonylmethyl, [N-(4-hydroxy-1,1-dioxidotetrahydro-3-thienyl)amino]-carbonylmethyl, (cyclopropylmethylamino)-carbonylmethyl, prop-2-yn-1-ylamino)-carbonylmethyl, (2-(morpholino)eth-1-ylamino)-carbonylmethyl, phenylsulfonylamino)-carbonylmethyl, [N-benzylamino]-carbonylmethyl, (N-(4-methylsulfonyl-benzyl)amino)-carbonylmethyl, tryptophanyl)-carbonylmethyl, (tyrosine)-carbonylmethyl, N-(1-carboxyprop-1-ylamino)-carbonylmethyl, (N-(2-carboxyeth-1-yl)-amino)-carbonylmethyl, N-(4-carboxybenzyl)-amino)-carbonylmethyl, N-[3-(N′-(4-(acrylic acid)-phenyl)carboxamido) pyrrolidin-3-yl]amino-carbonylmethyl, N-[4-(N′-(4-(acrylic acid)-phenyl)carboxamido) piperidin-4-yl]amino-carbonylmethyl, [2-(N,N-dimethylamino)eth-1-ylamino]-carbonylmethyl, [(1-(5-methyl-4H-1,2,4-triazol-3-yl)ethyl)amino]-carbonylmethyl, (1-methyl-1-[N-(1-methyl-2-carboxy-1H-indol-5-yl)aminocarbonyl]eth-1-ylamino-carbonylmethyl, [N-(1-methylpyrrolidin-3-yl-ethyl)-amino]-carbonylmethyl, (1-methyl-1-[N-(4-(acrylic acid)phenyl)aminocarbonyl]eth-1-ylamino-carbonylmethyl, (1-methyl-1-[N-(4-(2-carboxy-furan-5-yl)phenyl)aminocarbonyl]eth-1-ylamino-carbonylmethyl, (1-methyl-1-[N-(4-(4-carboxy-thiazol-2-yl)phenyl)aminocarbonyl]eth-1-ylamino-carbonylmethyl, (2-(4-methylpiperazin-1-yl)eth-1-ylamino)-carbonylmethyl, [(1-methylpyrrolidin-3-yl)methylamino]-carbonylmethyl, [N-(1-methylpiperidin-3-yl-methyl)-amino]-carbonylmethyl, (1-piperidin-1-ylcyclopentyl)methylamino]-carbonylmethyl, (1-(acetyl)-pyrrolidin-2-ylmethyl)amino)-carbonylmethyl, [(2-(N,N-dimethylamino)-carbonyl)methylamino]-carbonylmethyl, [N-(1,1-dioxidotetrahydro-3-thienyl)methylamino]-carbonylmethyl, (N-methyl-N-cyclohexyl-amino)-carbonylmethyl, (N-methyl-N-carboxymethyl-amino)-carbonylmethyl, [N-methyl-N-benzyl-amino]-carbonylmethyl, (N-methyl-N—(N′,N′-dimethylaminoacetyl)-amino)-carbonylmethyl, [N-methyl-N-phenyl-amino]-carbonylmethyl, (N-methyl-N-isopropyl-amino)-carbonylmethyl, (N-methyl-N—(N′-methylpiperidin-4-yl)amino)-carbonylmethyl, [N-methyl-N-(1-methylpiperidin-4-yl)amino]-carbonylmethyl, [N-methyl-N-(1-methylpiperidin-4-yl-methyl)-amino]-carbonylmethyl, [N-methyl-N-(1-methylpiperidin-3-yl-methyl)-amino]-carbonylmethyl, [N-methyl-N-(1-methylpyrazin-2-yl-methyl)-amino]-carbonylmethyl, [N-methyl-N-(5-methyl-1H-imidazol-2-ylmethyl)-amino]-carbonylmethyl, (N-methyl-N-[2-(hydroxy)eth-1-yl]amino)-carbonylmethyl, (N-methyl-N-[2-(N′,N′-dimethylamino)eth-1-yl]amino)-carbonylmethyl, N-methyl-N-[2-(N′,N′-diethylamino)eth-1-yl]amino)-carbonylmethyl, (N-methyl-N-[2-(pyridin-2-yl)eth-1-yl]amino)-carbonylmethyl, (N-methyl-N-[2-(pyridin-4-yl)eth-1-yl]amino)-carbonylmethyl, [N-methyl-N-(1-(1,3-thiazol-2-yl)ethyl)-amino]-carbonylmethyl, (N-methyl-N-[3-(N′,N′-dimethylamino)prop-1-yl]amino)-carbonylmethyl, (N-methyl-N-(1-carboxy-2-methylprop-1-yl)-amino)-carbonylmethyl, (N-ethyl-N-propyl-amino)-carbonylmethyl, (N-ethyl-N-[2-(methoxy)eth-1-yl]amino)-carbonylmethyl, (N-ethyl-N-[2-(N′,N′-diethylamino)eth-1-yl]amino)-carbonylmethyl, [7-methyl-2,7-diazaspiro[4.4]non-2-yl]-carbonylmethyl, (5-methyl-2,5-diazabicyclo[2.2.1]heptyl-2-yl)-carbonylmethyl, (4-methyl-1,4-diazepan-1-yl)-carbonylmethyl, (piperidinyl)-carbonylmethyl, (4-carboxy-piperidinyl)-carbonylmethyl, (3-carboxypiperidinyl)-carbonylmethyl, (4-hydroxypiperidinyl)-carbonylmethyl, (4-(2-hydroxyeth-1-yl)piperidin-1-yl)-carbonylmethyl, [4-(N,N-dimethylamino)-piperidin-1-yl]-carbonylmethyl, (3-(N,N-dimethylamino)-methylpiperidin-1-yl)-carbonylmethyl, (2-(2-(N,N-dimethylamino) -eth-1-yl)piperidin-1-yl)-carbonylmethyl, [4-(4-methyl-4H-1,2,4-triazol-3-yl) piperidin-1-yl]-carbonylmethyl, (4-pyrrolidinyl-piperidinyl)-carbonylmethyl, (3-pyrrolidinyl-piperidinyl)-carbonylmethyl, [4-(N,N-diethylamino)-piperidin-1-yl]-carbonylmethyl, (4-(azetidin-1-yl) -piperidin-1-yl)-carbonylmethyl, (4-(piperidin-1-yl)-piperidin-1-yl)-carbonylmethyl, (hexahydropyrrolo[1,2-a]pyrazin-2(1H)-yl)-carbonylmethyl, [(2-(N,N-dimethylamino)-methyl)morpholino]-carbonylmethyl, (3,5-dimethylmorpholino)-carbonylmethyl, (thiomorpholino)-carbonylmethyl, morpholino-carbonylmethyl, (pyrrolidinyl)-carbonylmethyl, (2-carboxy-pyrrolidin-1-yl)-carbonylmethyl, (2-(carboxy)-4-hydroxy-pyrrolidin-1-yl)-carbonylmethyl, (2-carboxamide-pyrrolidin-1-yl)-carbonylmethyl, (2-(N,N-dimethylaminocarbonyl)-pyrrolidin-1-yl)-carbonylmethyl, (3-(N′,N′-dimethylamino)-pyrrolidin-1-yl)-carbonylmethyl, (3-(N′,N′-diethylamino) -pyrrolidin-1-yl)-carbonylmethyl, (3-(pyridin-3-yl)-pyrrolidin-1-yl)-carbonylmethyl, (2-pyidin-4-ylpyrrolidin-1-yl)-carbonylmethyl, piperazin-1-yl-carbonylmethyl, (4-methylpiperazinyl)-carbonylmethyl, (4-(carboxymethyl)-piperazin-1-yl)-carbonylmethyl, (4-(2-hydroxyeth-1-yl)piperazin-1-yl)-carbonylmethyl, (4-(isopropyl)piperazin-1-yl)-carbonylmethyl, (4-(2-methoxyeth-1-yl)piperazin-1-yl)-carbonylmethyl, (4-(ethyl)piperazin-1-yl)-carbonylmethyl, (4-(N′,N′-dimethylaminoacetyl)-piperazin-1-yl)-carbonylmethyl, and (4-(6-methoxypyridin-2-yl) piperazin-1-yl)-carbonylmethyl.

In another embodiment, R is selected from morpholinocarbonylmethyl, N,N-dimethylaminocarbonylmethyl, (4-pyrrolidinyl-piperidin-1-yl)carbonylmethyl, piperazinylcarbonylmethyl. In some aspects, R is an oxide of morpholinocarbonylmethyl, N,N-dimethylaminocarbonylmethyl, (4-pyrrolidinyl-piperidin-1-yl)carbonylmethyl, piperazinylcarbonylmethyl.

In another embodiment, R is selected from [(N,N-dimethylamino)prop-2-en-1-yl]-carbonylmethyl, (N,N-dimethylpiperidin-4-aminium trifluoroacetate)acetyl, 2-(N,N-dimethylpiperidin-4-aminium trifluoroacetate)morpholino acetyl, (2-(diisopropyl)eth-1-yl)-carbonylmethyl, (pyridin-4-ylcarbonylhydrazino)-carbonylmethyl, (N-(4-carboxybenzyl)-amino)carbonylhydrazino)-carbonylmethyl, (acetylhydrazino)-carbonylmethyl, ((N′,N′-dimethylaminomethyl-carbonyl)hydrazino)-carbonylmethyl.

In still other embodiments, R is substituted alkyl, wherein said substituted alkyl is selected from the group consisting of aminoalkyl, substituted aminoalkyl, arylalkyl, substituted arylalkyl, heteroarylalkyl, substituted heteroarylalkyl, heterocyclylalkyl, substituted heterocyclylalkyl, —CH₂COOH, and —CH₂CONR¹²R¹³, wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

In other embodiments, R is —CH₂CONR¹²R¹³ and at least one of R¹² or R¹³ is alkyl, substituted alkyl, or heteroaryl. In some aspects at least one of R¹² or R¹³ is methyl, carboxymethyl, 2-hydroxyethyl, 2-morpholin-4-ylethyl, or tetrazoyl-5-yl. In other aspects R is 1-methyl-piperidin-4-yl, 1-methyl-piperidin-3-ylmethyl, and thiazol-2-yl carbamoyl methyl.

In yet other embodiments, R is —CH₂CONR¹²R¹³ and R¹² and R¹³ and the nitrogen atom to which they are attached form a substituted or unsubstituted heterocyclic ring. In some aspects R¹² and R¹³ and the nitrogen atom to which they are attached form a substituted or unsubstituted morpholino, substituted or unsubstituted piperidinyl, or a substituted or unsubstituted pyrrolidinyl ring. In other aspects the substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

In some embodiments, R¹² and R¹³ and the nitrogen atom to which they are attached together form a group selected from N,N-dimethylamino, N-(4-hydroxy-1,1-dioxidotetrahydro-3-thienyl)amino, cyclopropylmethylamino, prop-2-yn-1-ylamino, 2-(morpholino)eth-1-ylamino, phenylsulfonylamino, N-benzylamino, N-(4-methylsulfonyl-benzyl)amino, tryptophanyl, tyrosine, N-1-carboxyprop-1-ylamino, N-(2-carboxyeth-1-yl)-amino, N-(4-carboxybenzyl)-amino, N-[3-(N′-(4-(acrylic acid)-phenyl)carboxamido)pyrrolidin-3-yl]amino, N-[4-(N′-(4-(acrylic acid)-phenyl)carboxamido) piperidin-4-yl]amino, 2-(N,N-dimethylamino)eth-1-ylamino, (1-(5-methyl-4H-1,2,4-triazol-3-yl)ethyl)amino, 1-methyl-1-[N-(1-methyl-2-carboxy-1H-indol-5-yl)aminocarbonyl]eth-1-ylamino, N-(1-methylpyrrolidin-3-yl-ethyl)-amino, 1-methyl-1-[N-(4-(acrylic acid)phenyl)aminocarbonyl]eth-1-ylamino, 1-methyl-1-[N-(4-(2-carboxy-furan-5-yl)phenyl)aminocarbonyl]eth-1-ylamino, 1-methyl-1-[N-(4-(4-carboxy-thiazol-2-yl)phenyl)aminocarbonyl]eth-1-ylamino, 2-(4-methylpiperazin-1-yl)eth-1-ylamino, 1-methylpyrrolidin-3-yl)methylamino, N-(1-methylpiperidin-3-yl-methyl)-amino, (1-piperidin-1-ylcyclopentyl)methylamino, 1-(acetyl)-pyrrolidin-2-ylmethyl)amino, (2-(N,N-dimethylamino)-carbonyl)methylamino, N-(1,1-dioxidotetrahydro-3-thienyl)methylamino, N-methyl-N-cyclohexyl-amino, N-methyl-N-carboxymethyl-amino, N-methyl-N-benzyl-amino, N-methyl-N—(N′,N′-dimethylaminoacetyl)-amino, N-methyl-N-phenyl-amino, N-methyl-N-isopropyl-amino, N-methyl-N—N′-methylpiperidin-4-yl)amino, N-methyl-N-(1-methylpiperidin-4-yl)amino, N-methyl-N-(1-methylpiperidin-4-yl-methyl)-amino, N-methyl-N-(1-methylpiperidin-3-yl-methyl)-amino, N-methyl-N-(1-methylpyrazin-2-yl-methyl)-amino, N-methyl-N-(5-methyl-1H-imidazol-2-ylmethyl)-amino, N-methyl-N-[2-(hydroxy)eth-1-yl]amino, N-methyl-N-[2-(N′,N′-dimethylamino)eth-1-yl]amino, N-methyl-N-[2-(N′,N′-diethylamino)eth-1-yl]amino, N-methyl-N-[2-(pyridin-2-yl)eth-1-yl]amino, N-methyl-N-[2-(pyridin-4-yl)eth-1-yl]amino, N-methyl-N-(1-(1,3-thiazol-2-yl)ethyl)-amino, N-methyl-N-[3-(N′,N′-dimethylamino)prop-1-yl]amino, N-methyl-N-(1-carboxy-2-methylprop-1-yl)-amino, N-ethyl-N-propyl-amino, N-ethyl-N-[2-(methoxy)eth-1-yl]amino, N-ethyl-N-[2-(N′,N′-diethylamino)eth-1-yl]amino, 7-methyl-2,7-diazaspiro[4.4]non-2-yl, 5-methyl-2,5-diazabicyclo[2.2.1]heptyl-2-yl, 4-methyl-1,4-diazepan-1-yl, piperidinyl, 4-carboxy-piperidinyl, 3-carboxypiperidinyl, 4-hydroxypiperidinyl, 4-(2-hydroxyeth-1-yl)piperidin-1-yl, 4-(N,N-dimethylamino) -piperidin-1-yl, 3-(N,N-dimethylamino)-methylpiperidin-1-yl, 2-(2-(N,N-dimethylamino) -eth-1-yl)piperidin-1-yl, 4-(4-methyl-4H-1,2,4-triazol-3-yl)piperidin-1-yl, 4-pyrrolidinyl-piperidinyl, 3-pyrrolidinyl-piperidinyl, 4-(N,N-diethylamino)-piperidin-1-yl, 4-(azetidin-1-yl)-piperidin-1-yl, 4-(piperidin-1-yl)-piperidin-1-yl, hexahydropyrrolo[1,2-a]pyrazin-2(1H)-yl, (2-(N,N-dimethylamino)-methyl)morpholino, 3,5-dimethylmorpholino, thiomorpholino, morpholino, pyrrolidinyl, 2-carboxy-pyrrolidin-1-yl, 2-(carboxy)-4-hydroxy-pyrrolidin-1-yl, 2-carboxamide-pyrrolidin-1-yl, 2-(N,N-dimethylaminocarbonyl)-pyrrolidin-1-yl, 3-(N′,N′-dimethylamino) -pyrrolidin-1-yl, 3-(N′,N′-diethylamino)-pyrrolidin-1-yl, 3-(pyridin-3-yl) -pyrrolidin-1-yl, 2-pyidin-4-ylpyrrolidin-1-y, piperazin-1-yl, 4-methylpiperazinyl, 4-(carboxymethyl)-piperazin-1-yl, 4-(2-hydroxyeth-1-yl)piperazin-1-yl, 4-(isopropyl)piperazin-1-yl, 4-(2-methoxyeth-1-yl)piperazin-1-yl, 4-(ethyl)piperazin-1-yl, 4-(N′,N′-dimethylaminoacetyl)-piperazin-1-yl, 4-(6-methoxypyridin-2-yl)piperazin-1-yl, and 2-dimethylaminomethylmorpholin-4-yl.

In some embodiments HET is selected from quinolinylene and substituted quinolinylene. In another embodiment HET is selected from quinolinylene, isoquinolinylene, 7-methyl-quinolinylene, 7-trifluoromethyl-quinolinylene, 8-fluoro-quinolinylene and 7-fluoro-quinolinylene. In yet another embodiment HET is 2-[substituted]-quinolin-6-yl, 2-[substituted]-7-methyl-quinolinyl, 2-[substituted]-7-fluoro-quinolinyl, 2-[substituted]-7-trifluoromethyl-quinolinyl, and 2-[substituted]-8-fluoro-quinolinyl.

In some embodiments HET is 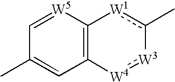
optionally substituted with (X)ₜ where X, t, W¹, W³, W⁴, and W⁵ are previously defined. In some aspects, W¹ is nitrogen. In other aspects where HET is selected from the group consisting of

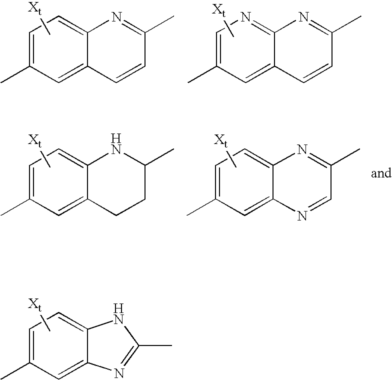

In some embodiments, HET is 1,4-phenylene optionally substituted with (X)ₜ where X and t are previously defined.

In some embodiments, t is 0.

In another embodiment, t is 1 and X is amino, nitro, methyl, or halo.

In other embodiments, HET is selected from the following groups:

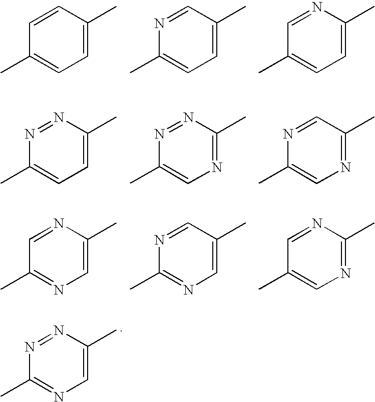

In some embodiments, Y is substituted aryl or substituted heteroaryl.

In some embodiments, Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized. In some embodiments Y is substituted 5-membered heteroaryl ring optionally fused to a phenyl ring and having one, two, or three heteroatoms independently selected from the group consisting of N, O, or S wherein the heteroatoms N or S are optionally oxidized.

In another embodiment —Y is —Ar¹-(G¹)_{q} where Ar¹ is selected from arylene and heteroarylene, G¹ is selected from halo, hydroxy, nitro, cyano, alkyl, substituted alkyl, alkoxy, substituted alkoxy, acyl, acylamino, aminoacyl, amino, substituted amino, carboxy and carboxy ester; and q is an integer from 1 to 3. In another embodiment where —Y is —Ar¹-(G¹)_{q}, Ar¹ is selected from phenyl, thiazolyl, furanyl, thienyl, pyridinyl, pyrazinyl, oxazolyl, isoxazolyl, pyrrolyl, imidazolyl, and pyrrolidinyl. In another embodiment where —Y is —Ar¹-(G¹)_{q}, G¹ is selected from bromo, chloro, methyl, hydroxy, methoxy, ethoxy, acetyl, acetamido, carboxy, and amino. In another embodiment Y is selected from 2,4-dimethylthiazol-5-yl, 3-bromo-4-aminophenyl, 3-amido-4-hydroxy-phenyl, 2-hydroxy-6-methoxy-phenyl, 4-(acetylamino)-phenyl, 2,4-dihydroxyphenyl, 2,4-dimethoxy-6-hydroxyphenyl, and 7-hydroxybenzofuranyl.

In another embodiment Y is —Ar¹—Ar²— where the —Ar¹—Ar²— group is selected from the group consisting of -aryl-aryl, -aryl-substituted aryl, -substituted aryl-aryl, -substituted aryl-substituted aryl, -aryl-heteroaryl, -aryl-substituted heteroaryl, -substituted aryl-heteroaryl, -substituted aryl-substituted heteroaryl, heteroaryl-aryl, heteroaryl-substituted aryl, substituted heteroaryl-aryl, substituted heteroaryl-substituted aryl, -aryl-cycloalkyl, -aryl-substituted cycloalkyl, -substituted aryl-cycloalkyl, -substituted aryl-substituted cycloalkyl, -aryl-heterocyclic, aryl-substituted heterocyclic, substituted aryl-heterocyclic, and substituted aryl-substituted heterocyclic.

In another embodiment where Y is —Ar¹—Ar²—, the —Ar¹—Ar²— group is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl) biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphenyl, 4-methoxybiphenyl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

In another embodiment where Y is —Ar¹—Ar²—, the —Ar¹—Ar²— group is selected from the group consisting of 4-(1H-imidazol-1-yl)phenyl, 2-furan-2-yl-5-methoxyphenyl, 5-methoxy-2-thiophen-2-ylphenyl, 2-(2,4-dimethoxypyrimidin-5-yl)-4-methoxyphenyl, 2-(pyrid-4-yl)phenyl, 3-amino-5-phenylthiophen-2-yl, 5-(4-chlorophenyl)-2-methylfuran-2-yl, 3-(4-chlorophenyl)-5-methylisoxazol-4-yl, 2-(4-chlorophenyl)-4-methylthiazol-5-yl, 3-(3,4-dichloro-phenyl)isoxazol-5-yl, 3,5-dimethyl-1-phenyl-1H-pyrazol-4-yl, 5-methyl-2-phenylthiophen-3-yl, and 1-phenyl-1H-pyrazol-4-yl.

In another embodiment where Y is —Ar¹—Ar²—, the —Ar¹—Ar²— group is selected from the group consisting of 2-cyclohexyl-N,N-dimethylamino-carbonylmethyl-5-methoxyphenyl, and 4-morpholinophenyl.

In still other embodiments, Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl. In some aspects, Y is substituted with one to three substitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester. In still other aspects, Y is 2,4-dimethylthiazol-5-yl.

In some embodiments, Y is selected from the corresponding Y groups in Table 1.

In some embodiments, -Het-Y is:

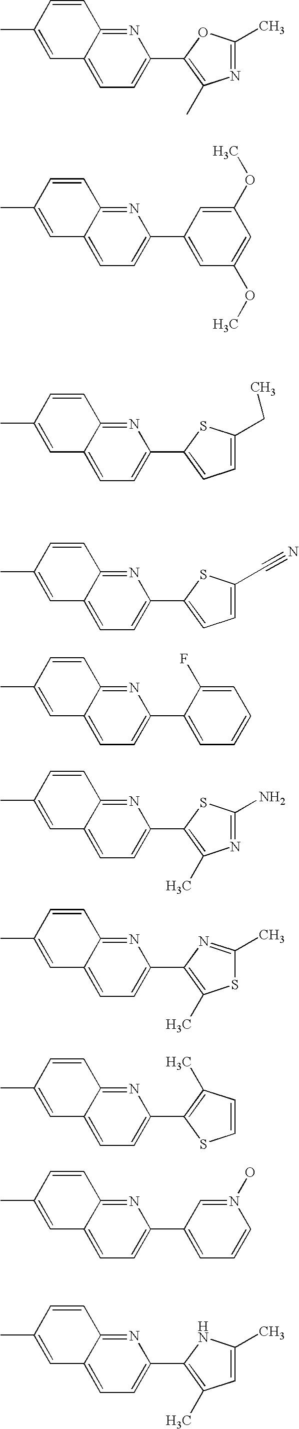

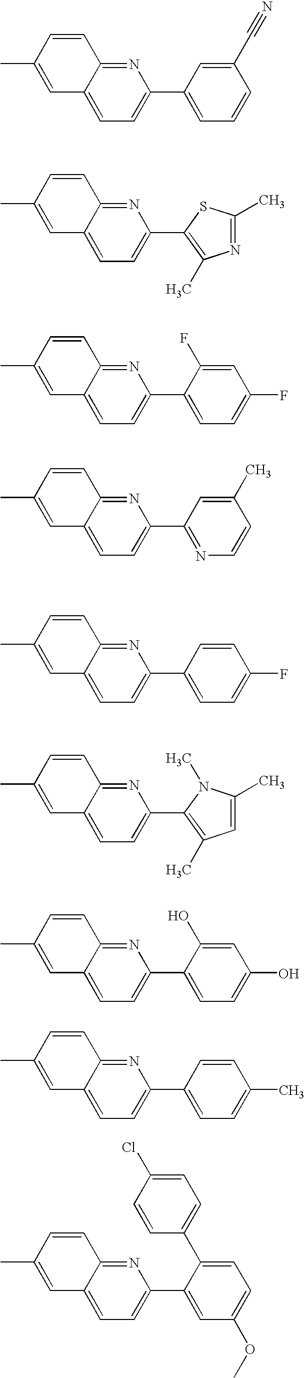

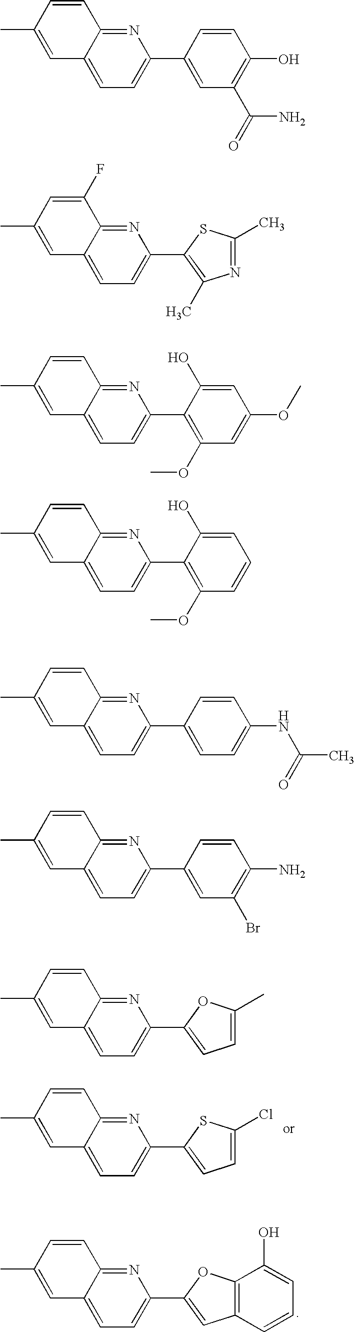

Preferred compounds of this invention or the pharmaceutically acceptable salts, partial salts, or tautomers thereof include those set forth in Table I below:

TABLE I

| Cmpd. | Structure | Name |
|---|---|---|
| 1 | 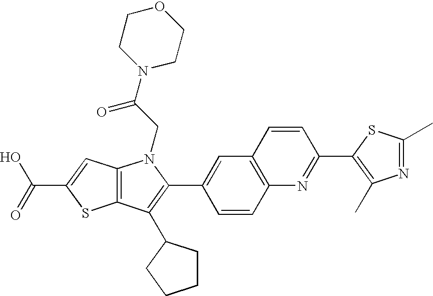 | 6-Cyclopentyl-5-[2-(2,4-dimethyl- thiazol-5-yl)-quinolin-6-yl]-4-(2- morpholin-4-yl-2-oxo-ethyl)-4H- thieno[3,2-b]pyrrole-2-carboxylic acid |
| 2 | 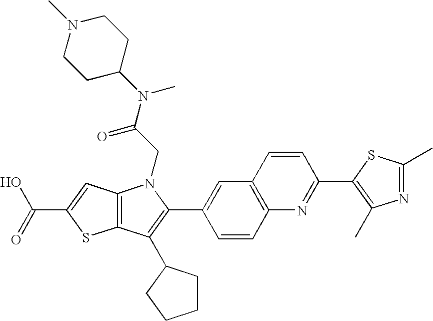 | 6-Cyclopentyl-5-[2-(2,4-dimethyl- thiazol-5-yl)-quinolin-6-yl]-4- {[methyl-(1-methyl-piperidin-4-yl)- carbamoyl]-methyl}-4H- thieno[3,2-b]pyrrole-2-carboxylic acid |
| 3 | 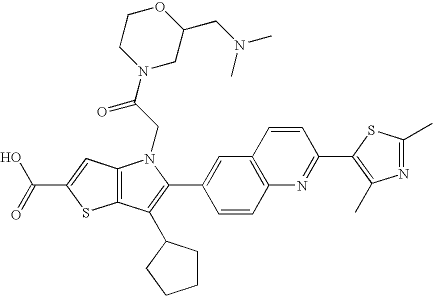 | 6-Cyclopentyl-4-[2-(2- dimethylaminomethyl-morpholin- 4-yl)-2-oxo-ethyl]-5-[2-(2,4- dimethyl-thiazol-5-yl)-quinolin-6- yl]-4H-thieno[3,2-b]pyrrole-2- carboxylic acid |
| 4 | 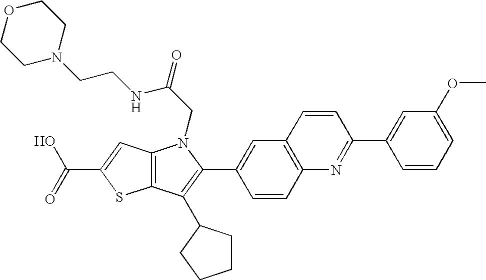 | 6-Cyclopentyl-5-[2-(3-methoxy- phenyl)-quinolin-6-yl]-4-[(2- morpholin-4-yl-ethylcarbamoyl)- methyl]-4H-thieno[3,2-b]pyrrole-2- carboxylic acid |
| 5 | 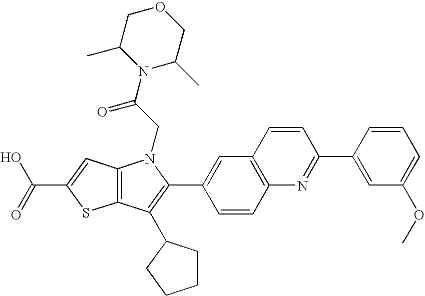 | 6-Cyclopentyl-4-[2-(3,5-dimethyl- morpholin-4-yl)-2-oxo-ethyl]-5-[2- (3-methoxy-phenyl)-quinolin-6-yl]- 4H-thieno[3,2-b]pyrrole-2- carboxylic acid |
| 6 | 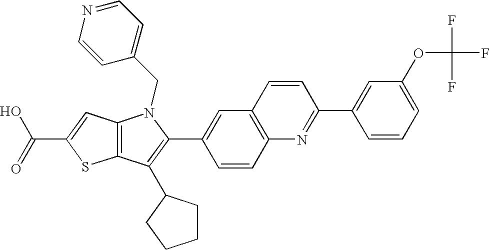 | 6-Cyclopentyl-4-pyridin-4- ylmethyl-5-[2-(3-trifluoromethoxy- phenyl)-quinolin-6-yl]-4H- thieno[3,2-b]pyrrole-2-carboxylic acid |
| 7 | 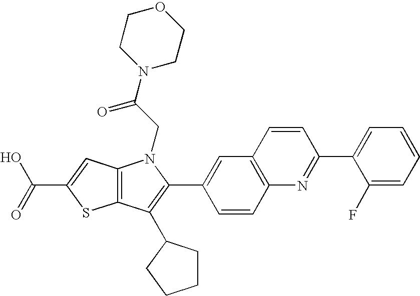 | 6-Cyclopentyl-5-[2-(2-fluoro- phenyl)-quinolin-6-yl]-4-(2- morpholin-4-yl-2-oxo-ethyl)-4H- thieno[3,2-b]pyrrole-2-carboxylic acid |
| 8 | 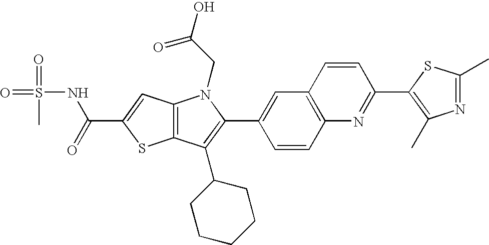 | {6-Cyclohexyl-5-[2-(2,4-dimethyl- thiazol-5-yl)-quinolin-6-yl]-2- methanesulfonylaminocarbonyl- thieno[3,2-b]pyrrol-4-yl}-acetic acid |
| 9 | 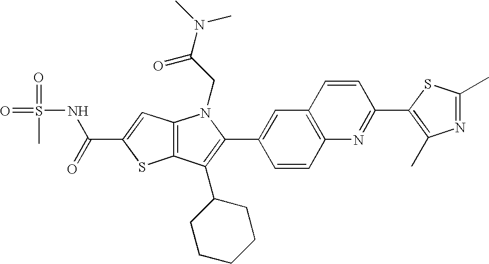 | 2-{6-Cyclohexyl-5-[2-(2,4- dimethyl-thiazol-5-yl)-quinolin-6- yl]-2- methanesulfonylaminocarbonyl- thieno[3,2-b]pyrrol-4-yl}-N,N- dimethyl-acetamide |
| 10 | 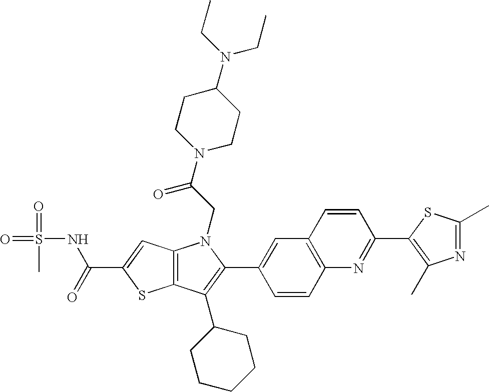 | N-{6-Cyclohexyl-4-[2-(4- diethylamino-piperidin-1-yl)-2- oxo-ethyl]-5-[2-(2,4-dimethyl- thiazol-5-yl)-quinolin-6-yl]-4H- thieno[3,2-b]pyrrole-2-carbonyl}- methanesulfonamide |
| 11 | 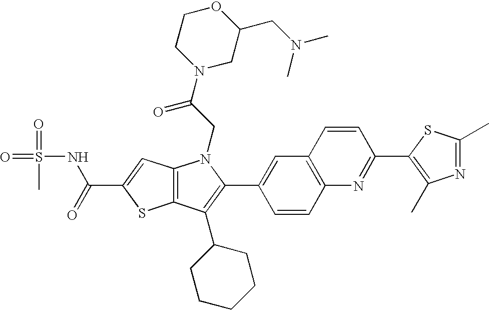 | N-{6-Cyclohexyl-4-[2-(2- dimethylaminomethyl-morpholin- 4-yl)-2-oxo-ethyl]-5-[2-(2,4- dimethyl-thiazol-5-yl)-quinolin-6- yl]-4H-thieno[3,2-b]pyrrole-2- carbonyl}-methanesulfonamide |
| 12 | 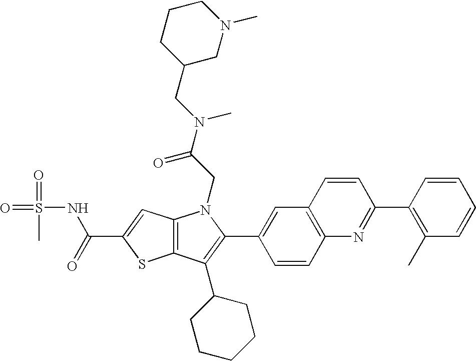 | 2-[6-Cyclohexyl-2- methanesulfonylaminocarbonyl-5- (2-o-tolyl-quinolin-6-yl)-thieno[3,2- b]pyrrol-4-yl]-N-methyl-N-(1- methyl-piperidin-3-ylmethyl)- acetamide |
| 13 | 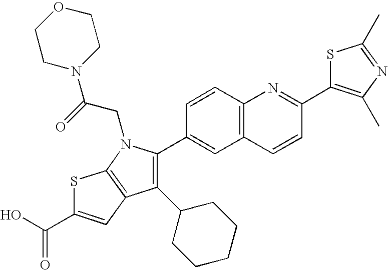 | 4-Cyclohexyl-5-[2-(2,4-dimethyl- thiazol-5-yl)-quinolin-6-yl]-6-(2- morpholin-4-yl-2-oxo-ethyl)-6H- thieno[2,3-b]pyrrole-2-carboxylic acid |
| 14 | 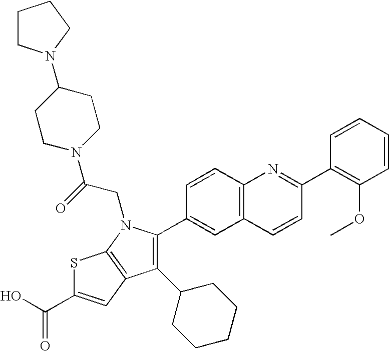 | 4-Cyclohexyl-5-[2-(2-methoxy- phenyl)-quinolin-6-yl]-6-[2-oxo-2- (4-pyrrolidin-1-yl-piperidin-1-yl)- ethyl]-6H-thieno[2,3-b]pyrrole-2- carboxylic acid |
| 15 | 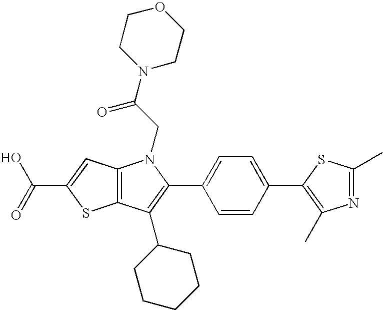 | 6-Cyclohexyl-5-[4-(2,4-dimethyl- thiazol-5-yl)-phenyl]-4-(2- morpholin-4-yl-2-oxo-ethyl)-4H- thieno[3,2-b]pyrrole-2-carboxylic acid |
| 16 | 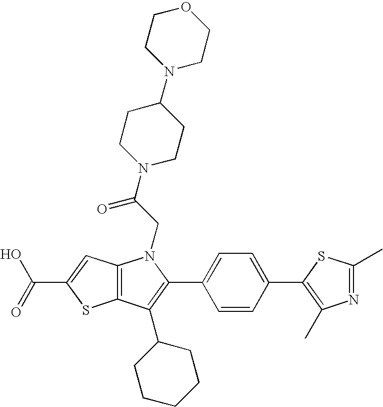 | 6-Cyclohexyl-5-[4-(2,4-dimethyl- thiazol-5-yl)-phenyl]-4-[2-(4- morpholin-4-yl-piperidin-1-yl)-2- oxo-ethyl]-4H-thieno[3,2- b]pyrrole-2-carboxylic acid |
| 17 | 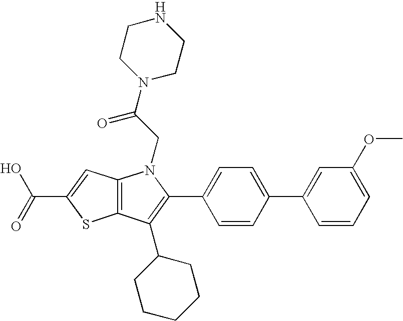 | 6-Cyclohexyl-5-(3′-methoxy- biphenyl-4-yl)-4-(2-oxo-2- piperazin-1-yl-ethyl)-4H- thieno[3,2-b]pyrrole-2-carboxylic acid |
| 18 | 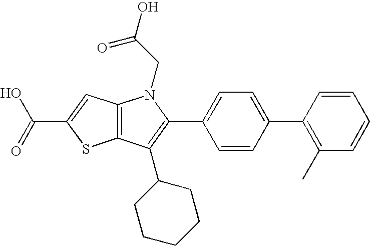 | 4-Carboxymethyl-6-cyclohexyl-5- (2′-methyl-biphenyl-4-yl)-4H- thieno[3,2-b]pyrrole-2-carboxylic acid |
| 19 | 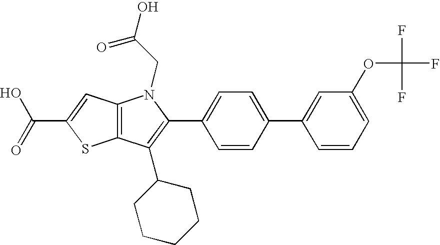 | 4-Carboxymethyl-6-cyclohexyl-5- (3′-trifluoromethoxy-biphenyl-4- yl)-4H-thieno[3,2-b]pyrrole-2- carboxylic acid |
| 20 | 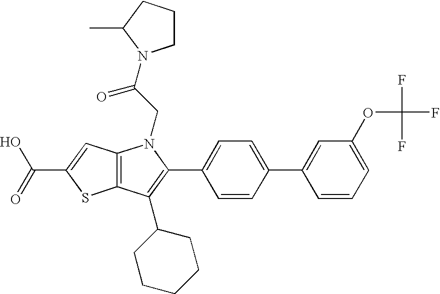 | 6-Cyclohexyl-4-[2-(2-methyl- pyrrolidin-1-yl)-2-oxo-ethyl]-5-(3′- trifluoromethoxy-biphenyl-4-yl)- 4H-thieno[3,2-b]pyrrole-2- carboxylic acid |
| 21 | 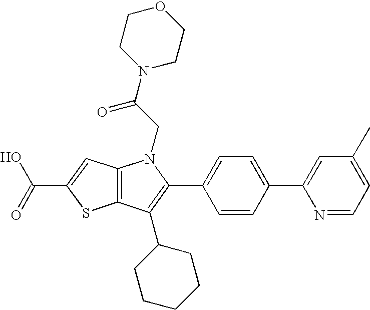 | 6-Cyclohexyl-5-[4-(4-methyl- pyridin-2-yl)-phenyl]-4-(2- morpholin-4-yl-2-oxo-ethyl)-4H- thieno[3,2-b]pyrrole-2-carboxylic acid |
| 22 | 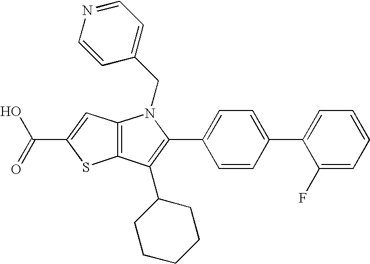 | 6-Cyclohexyl-5-(2′-fluoro- biphenyl-4-yl)-4-pyridin-4- ylmethyl-4H-thieno[3,2-b]pyrrole- 2-carboxylic acid |
| 23 | 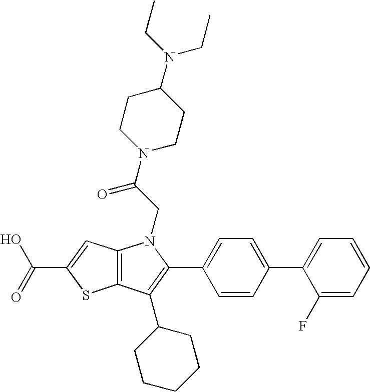 | 6-Cyclohexyl-4-[2-(4- diethylamino-piperidin-1-yl)-2- oxo-ethyl]-5-(2′-fluoro-biphenyl-4- yl)-4H-thieno[3,2-b]pyrrole-2- carboxylic acid |
| 24 | 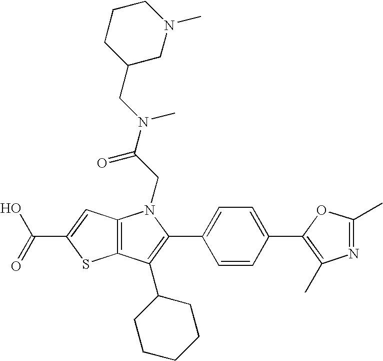 | 6-Cyclohexyl-5-[4-(2,4-dimethyl- oxazol-5-yl)-phenyl]-4-{[methyl-(1- methyl-piperidin-3-ylmethyl)- carbamoyl]-methyl}-4H- thieno[3,2-b]pyrrole-2-carboxylic acid |
| 25 | 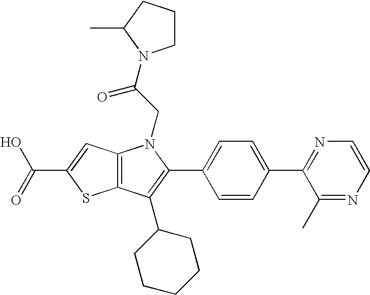 | 6-Cyclohexyl-5-[4-(3-methyl- pyrazin-2-yl)-phenyl]-4-[2-(2- methyl-pyrrolidin-1-yl)-2-oxo- ethyl]-4H-thieno[3,2-b]pyrrole-2- carboxylic acid |
| 26 | 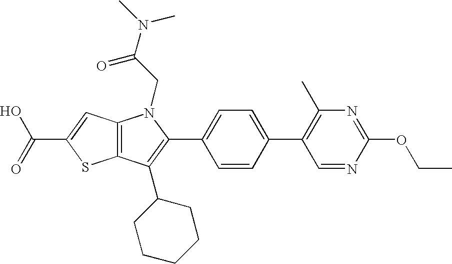 | 6-Cyclohexyl-4- dimethylcarbamoylmethyl-5-[4-(2- ethoxy-4-methyl-pyrimidin-5-yl)- phenyl]-4H-thieno[3,2-b]pyrrole-2- carboxylic acid |
| 27 | 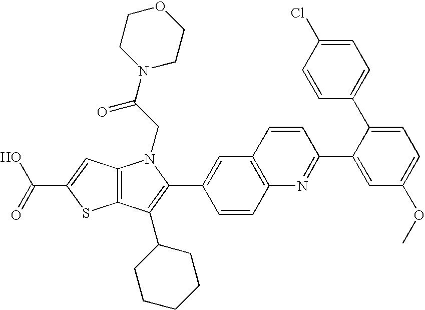 | 5-[2-(4′-Chloro-4-methoxy- biphenyl-2-yl)-quinolin-6-yl]-6- cyclohexyl-4-(2-morpholin-4-yl-2- oxo-ethyl)-4H-thieno[3,2- b]pyrrole-2-carboxylic acid |
| 28 | 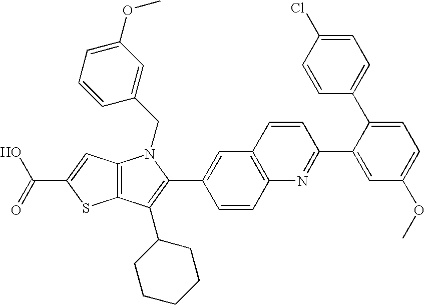 | 5-[2-(4′-Chloro-4-methoxy- biphenyl-2-yl)-quinolin-6-yl]-6- cyclohexyl-4-(3-methoxy-benzyl)- 4H-thieno[3,2-b]pyrrole-2- carboxylic acid |
| 29 | 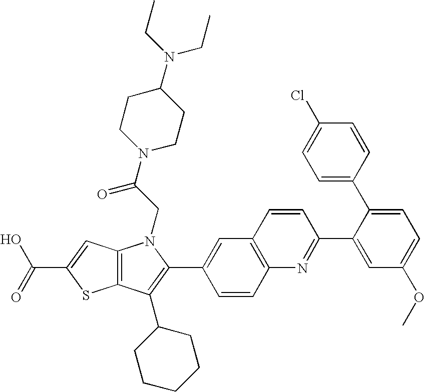 | 5-[2-(4′-Chloro-4-methoxy- biphenyl-2-yl)-quinolin-6-yl]-6- cyclohexyl-4-[2-(4-diethylamino- piperidin-1-yl)-2-oxo-ethyl]-4H- thieno[3,2-b]pyrrole-2-carboxylic acid |
| 30 | 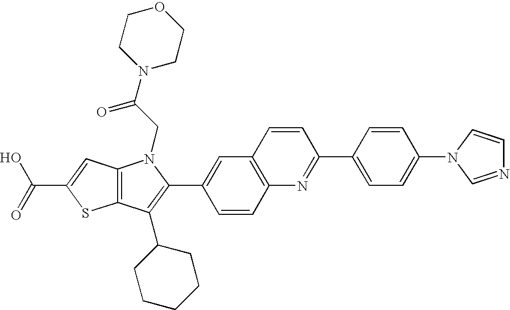 | 6-Cyclohexyl-5-[2-(4-imidazol-1- yl-phenyl)-quinolin-6-yl]-4-(2- morpholin-4-yl-2-oxo-ethyl)-4H- thieno[3,2-b]pyrrole-2-carboxylic acid |
| 31 | 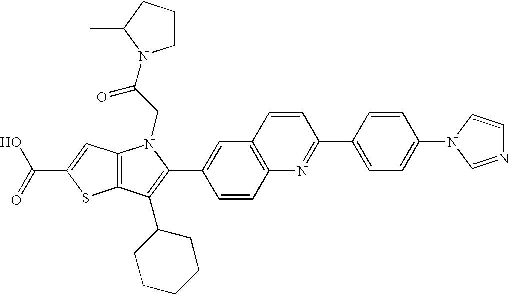 | 6-Cyclohexyl-5-[2-(4-imidazol-1- yl-phenyl)-quinolin-6-yl]-4-[2-(2- methyl-pyrrolidin-1-yl)-2-oxo- ethyl]-4H-thieno[3,2-b]pyrrole-2- carboxylic acid |
| 32 | 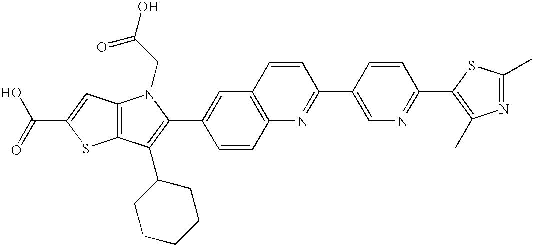 | 4-Carboxymethyl-6-cyclohexyl-5- [2-(2,4-dimethyl-thiazol-5-yl)- quinolin-6-yl]-4H-thieno[3,2- b]pyrrole-2-carboxylic acid |
| 33 | 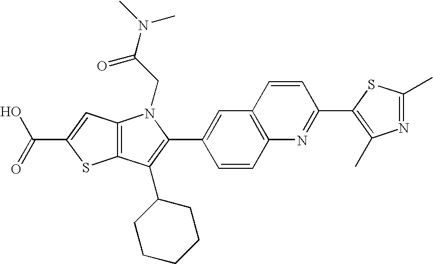 | 6-Cyclohexyl-4- dimethylcarbamoylmethyl-5-[2- (2,4-dimethyl-thiazol-5-yl)- quinolin-6-yl]-4H-thieno[3,2- b]pyrrole-2-carboxylic acid |
| 34 | 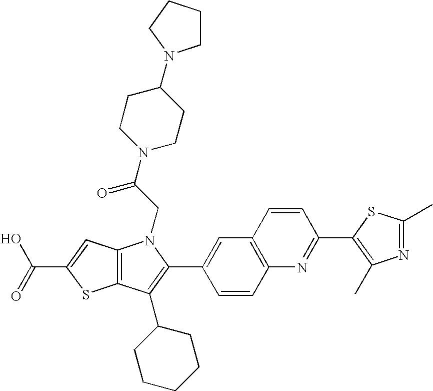 | 6-Cyclohexyl-5-[2-(2,4-dimethyl- thiazol-5-yl)-quinolin-6-yl]-4-[2- oxo-2-(4-pyrrolidin-1-yl-piperidin- 1-yl)-ethyl]-4H-thieno[3,2- b]pyrrole-2-carboxylic acid |
| 35 | 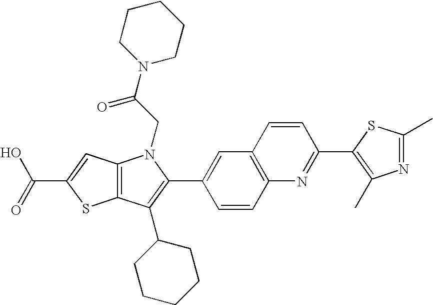 | 6-Cyclohexyl-5-[2-(2,4-dimethyl- thiazol-5-yl)-quinolin-6-yl]-4-(2- oxo-2-piperidin-1-yl-ethyl)-4H- thieno[3,2-b]pyrrole-2-carboxylic acid |
| 36 | 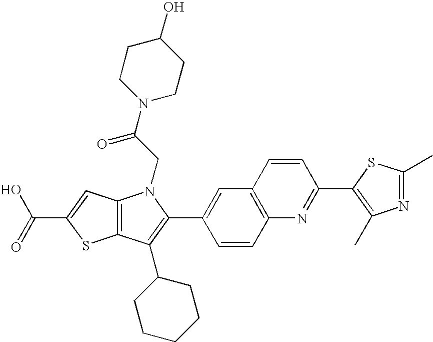 | 6-Cyclohexyl-5-[2-(2,4-dimethyl- thiazol-5-yl)-quinolin-6-yl]-4-[2-(4- hydroxy-piperidin-1-yl)-2-oxo- ethyl]-4H-thieno[3,2-b]pyrrole-2- carboxylic acid |
| 37 | 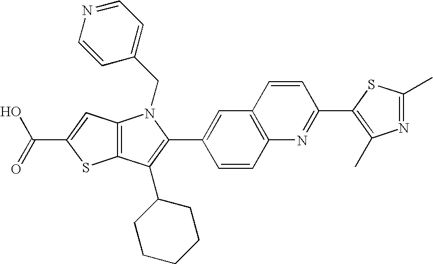 | 6-Cyclohexyl-5-[2-(2,4-dimethyl- thiazol-5-yl)-quinolin-6-yl]-4- pyridin-4-ylmethyl-4H-thieno[3,2- b]pyrrole-2-carboxylic acid |
| 38 | 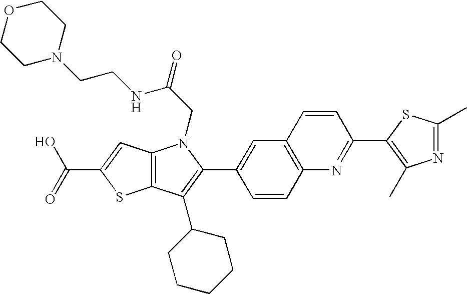 | 6-Cyclohexyl-5-[2-(2,4-dimethyl- thiazol-5-yl)-quinolin-6-yl]-4-[(2- morpholin-4-yl-ethylcarbamoyl)- methyl]-4H-thieno[3,2-b]pyrrole-2- carboxylic acid |
| 39 | 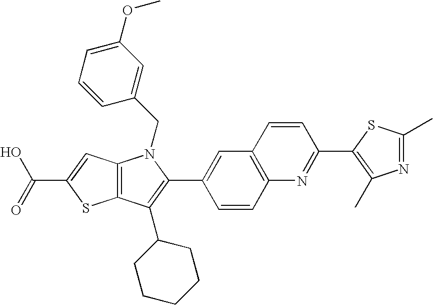 | 6-Cyclohexyl-5-[2-(2,4-dimethyl- thiazol-5-yl)-quinolin-6-yl]-4-(3- methoxy-benzyl)-4H-thieno[3,2- b]pyrrole-2-carboxylic acid |
| 40 | 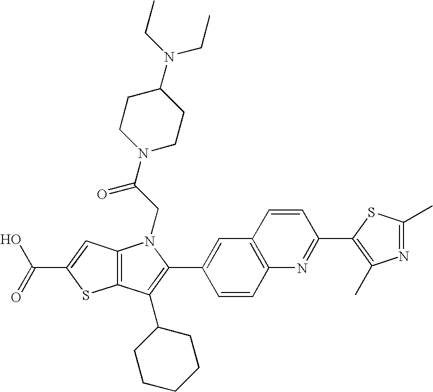 | 6-Cyclohexyl-4-[2-(4- diethylamino-piperidin-1-yl)-2- oxo-ethyl]-5-[2-(2,4-dimethyl- thiazol-5-yl)-quinolin-6-yl]-4H- thieno[3,2-b]pyrrole-2-carboxylic acid |
| 41 | 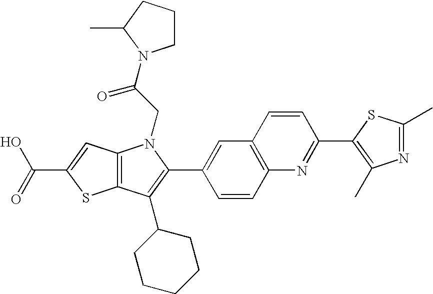 | 6-Cyclohexyl-5-[2-(2,4-dimethyl- thiazol-5-yl)-quinolin-6-yl]-4-[2-(2- methyl-pyrrolidin-1-yl)-2-oxo- ethyl]-4H-thieno[3,2-b]pyrrole-2- carboxylic acid |
| 42 | 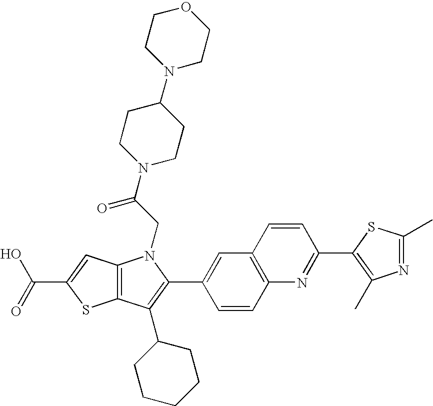 | 6-Cyclohexyl-5-[2-(2,4-dimethyl- thiazol-5-yl)-quinolin-6-yl]-4-[2-(4- morpholin-4-yl-piperidin-1-yl)-2- oxo-ethyl]-4H-thieno[3,2- b]pyrrole-2-carboxylic acid |
| 43 | 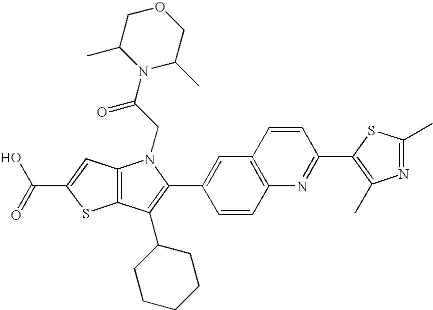 | 6-Cyclohexyl-4-[2-(3,5-dimethyl- morpholin-4-yl)-2-oxo-ethyl]-5-[2- (2,4-dimethyl-thiazol-5-yl)- quinolin-6-yl]-4H-thieno[3,2- b]pyrrole-2-carboxylic acid |
| 44 | 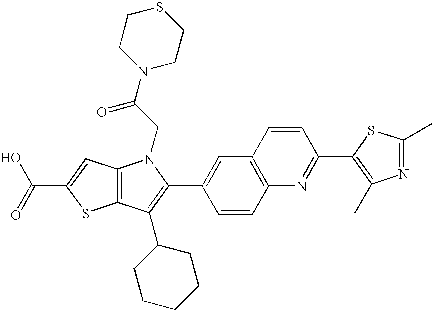 | 6-Cyclohexyl-5-[2-(2,4-dimethyl- thiazol-5-yl)-quinolin-6-yl]-4-(2- oxo-2-thiomorpholin-4-yl-ethyl)- 4H-thieno[3,2-b]pyrrole-2- carboxylic acid |
| 45 | 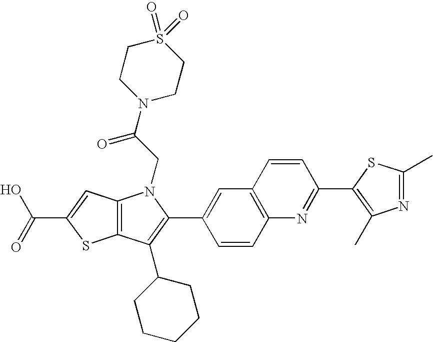 | 6-Cyclohexyl-5-[2-(2,4-dimethyl- thiazol-5-yl)-quinolin-6-yl]-4-[2- (1,1-dioxo-1-thiomorpholin-4-yl)- 2-oxo-ethyl]-4H-thieno[3,2- b]pyrrole-2-carboxylic acid |
| 46 | 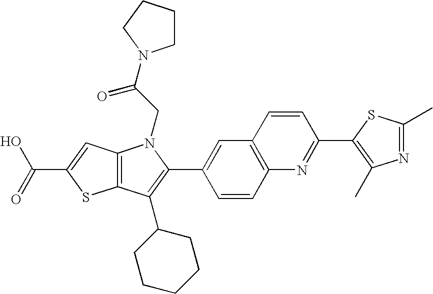 | 6-Cyclohexyl-5-[2-(2,4-dimethyl- thiazol-5-yl)-quinolin-6-yl]-4-(2- oxo-2-pyrrolidin-1-yl-ethyl)-4H- thieno[3,2-b]pyrrole-2-carboxylic acid |
| 47 | 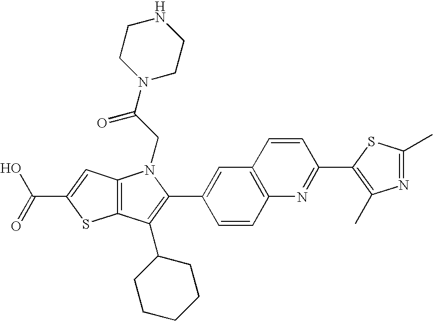 | 6-Cyclohexyl-5-[2-(2,4-dimethyl- thiazol-5-yl)-quinolin-6-yl]-4-(2- oxo-2-piperazin-1-yl-ethyl)-4H- thieno[3,2-b]pyrrole-2-carboxylic acid |
| 48 | 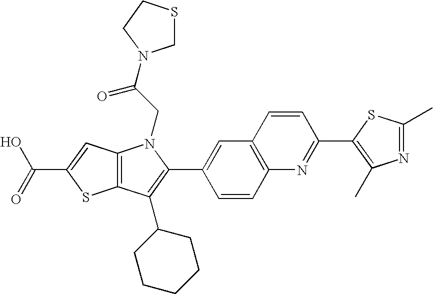 | 6-Cyclohexyl-5-[2-(2,4-dimethyl- thiazol-5-yl)-quinolin-6-yl]-4-(2- oxo-2-thiazolidin-3-yl-ethyl)-4H- thieno[3,2-b]pyrrole-2-carboxylic acid |
| 49 | 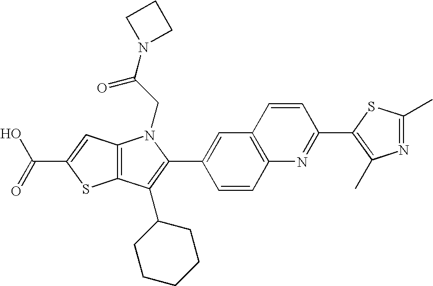 | 4-(2-Azetidin-1-yl-2-oxo-ethyl)-6- cyclohexyl-5-[2-(2,4-dimethyl- thiazol-5-yl)-quinolin-6-yl]-4H- thieno[3,2-b]pyrrole-2-carboxylic acid |
| 50 | 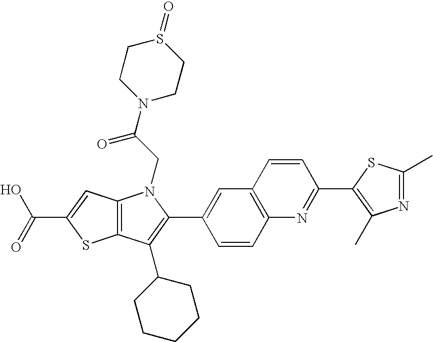 | 6-Cyclohexyl-5-[2-(2,4-dimethyl- thiazol-5-yl)-quinolin-6-yl]-4-[2- oxo-2-(1-oxo-1-thiomorpholin-4- yl)-ethyl]-4H-thieno[3,2-b]pyrrole- 2-carboxylic acid |
| 51 | 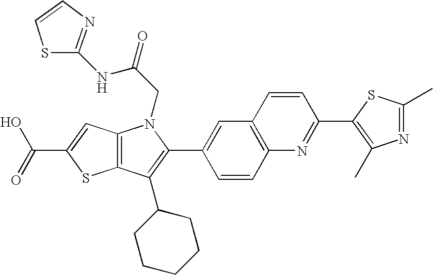 | 6-Cyclohexyl-5-[2-(2,4-dimethyl- thiazol-5-yl)-quinolin-6-yl]-4- (thiazol-2-ylcarbamoylmethyl)-4H- thieno[3,2-b]pyrrole-2-carboxylic acid |
| 52 | 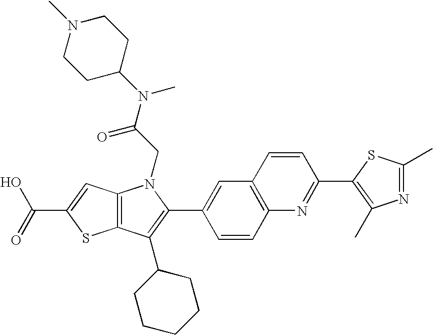 | 6-Cyclohexyl-5-[2-(2,4-dimethyl- thiazol-5-yl)-quinolin-6-yl]-4- {[methyl-(1-methyl-piperidin-4-yl)- carbamoyl]-methyl}-4H- thieno[3,2-b]pyrrole-2-carboxylic acid |
| 53 | 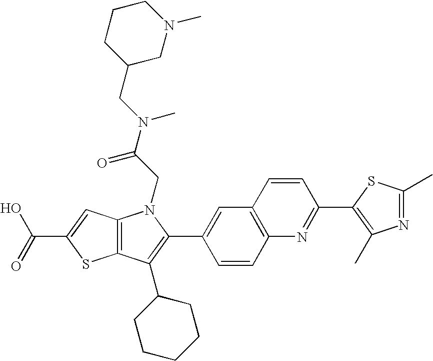 | 6-Cyclohexyl-5-[2-(2,4-dimethyl- thiazol-5-yl)-quinolin-6-yl]-4- {[methyl-(1-methyl-piperidin-3- ylmethyl)-carbamoyl]-methyl}-4H- thieno[3,2-b]pyrrole-2-carboxylic acid |
| 54 | 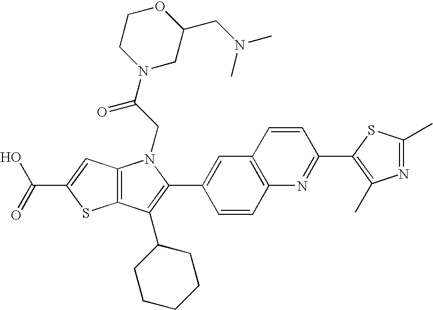 | 6-Cyclohexyl-4-[2-(2- dimethylaminomethyl-morpholin- 4-yl)-2-oxo-ethyl]-5-[2-(2,4- dimethyl-thiazol-5-yl)-quinolin-6- yl]-4H-thieno[3,2-b]pyrrole-2- carboxylic acid |
| 55 | 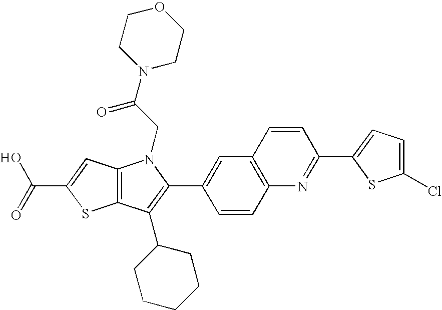 | 5-[2-(5-Chloro-thiophen-2-yl)- quinolin-6-yl]-6-cyclohexyl-4-(2- morpholin-4-yl-2-oxo-ethyl)-4H- thieno[3,2-b]pyrrole-2-carboxylic acid |
| 56 | 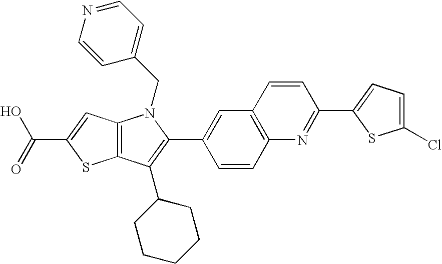 | 5-[2-(5-Chloro-thiophen-2-yl)- quinolin-6-yl]-6-cyclohexyl-4- pyridin-4-ylmethyl-4H-thieno[3,2- b]pyrrole-2-carboxylic acid |
| 57 | 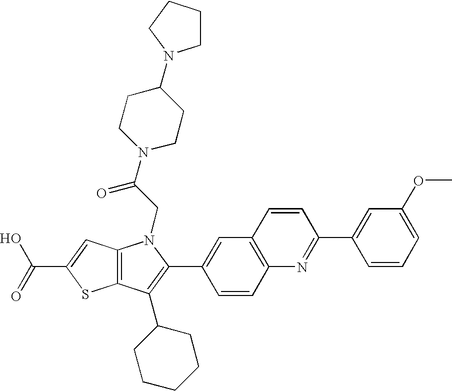 | 6-Cyclohexyl-5-[2-(3-methoxy- phenyl)-quinolin-6-yl]-4-[2-oxo-2- (4-pyrrolidin-1-yl-piperidin-1-yl)- ethyl]-4H-thieno[3,2-b]pyrrole-2- carboxylic acid |
| 58 | 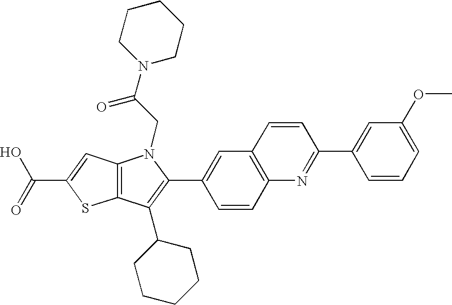 | 6-Cyclohexyl-5-[2-(3-methoxy- phenyl)-quinolin-6-yl]-4-(2-oxo-2- piperidin-1-yl-ethyl)-4H- thieno[3,2-b]pyrrole-2-carboxylic acid |
| 59 | 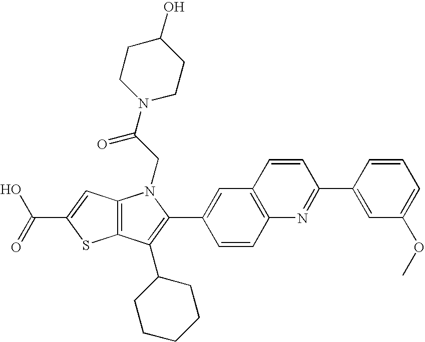 | 6-Cyclohexyl-4-[2-(4-hydroxy- piperidin-1-yl)-2-oxo-ethyl]-5-[2- (3-methoxy-phenyl)-quinolin-6-yl]- 4H-thieno[3,2-b]pyrrole-2- carboxylic acid |
| 60 | 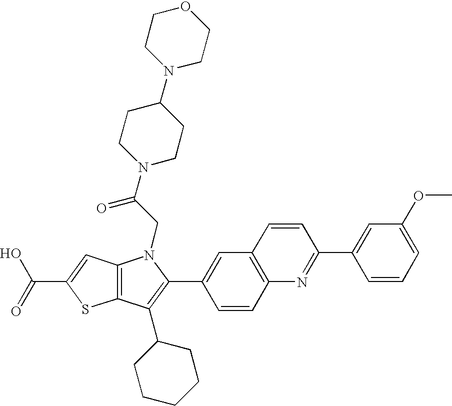 | 6-Cyclohexyl-5-[2-(3-methoxy- phenyl)-quinolin-6-yl]-4-[2-(4- morpholin-4-yl-piperidin-1-yl)-2- oxo-ethyl]-4H-thieno[3,2- b]pyrrole-2-carboxylic acid |
| 61 | 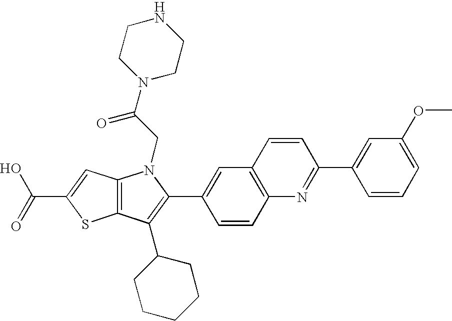 | 6-Cyclohexyl-5-[2-(3-methoxy- phenyl)-quinolin-6-yl]-4-(2-oxo-2- piperazin-1-yl-ethyl)-4H- thieno[3,2-b]pyrrole-2-carboxylic acid |
| 62 | 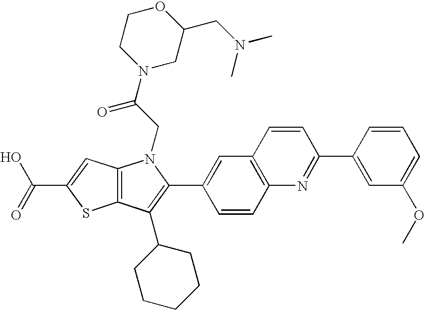 | 6-Cyclohexyl-4-[2-(2- dimethylaminomethyl-morpholin- 4-yl)-2-oxo-ethyl]-5-[2-(3- methoxy-phenyl)-quinolin-6-yl]- 4H-thieno[3,2-b]pyrrole-2- carboxylic acid |
| 63 | 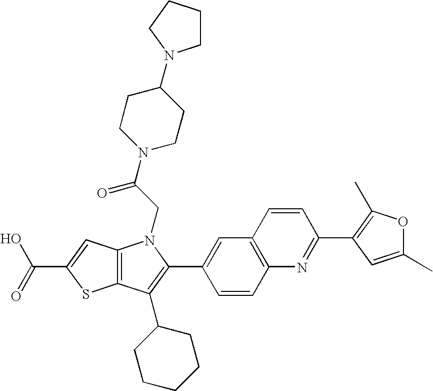 | 6-Cyclohexyl-5-[2-(2,5-dimethyl- furan-3-yl)-quinolin-6-yl]-4-[2-oxo- 2-(4-pyrrolidin-1-yl-piperidin-1-yl)- ethyl]-4H-thieno[3,2-b]pyrrole-2- carboxylic acid |
| 64 | 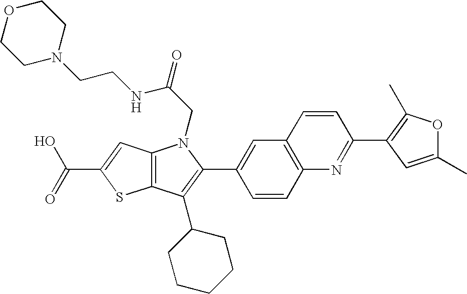 | 6-Cyclohexyl-5-[2-(2,5-dimethyl- furan-3-yl)-quinolin-6-yl]-4-[(2- morpholin-4-yl-ethylcarbamoyl)- methyl]-4H-thieno[3,2-b]pyrrole-2- carboxylic acid |
| 65 | 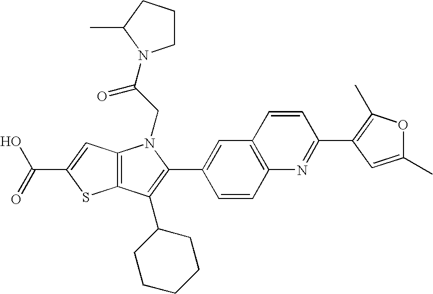 | 6-Cyclohexyl-5-[2-(2,5-dimethyl- furan-3-yl)-quinolin-6-yl]-4-[2-(2- methyl-pyrrolidin-1-yl)-2-oxo- ethyl]-4H-thieno[3,2-b]pyrrole-2- carboxylic acid |
| 66 | 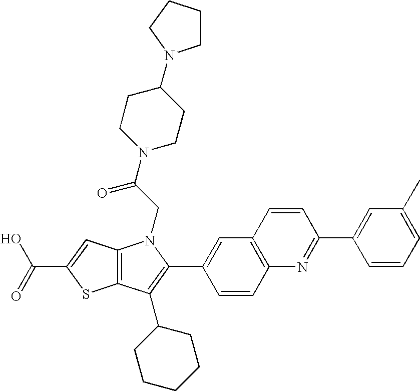 | 6-Cyclohexyl-4-[2-oxo-2-(4- pyrrolidin-1-yl-piperidin-1-yl)- ethyl]-5-(2-m-tolyl-quinolin-6-yl)- 4H-thieno[3,2-b]pyrrole-2- carboxylic acid |
| 67 | 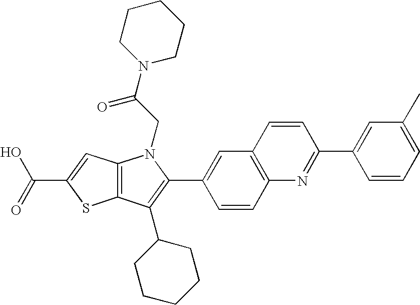 | 6-Cyclohexyl-4-(2-oxo-2-piperidin- 1-yl-ethyl)-5-(2-m-tolyl-quinolin-6- yl)-4H-thieno[3,2-b]pyrrole-2- carboxylic acid |
| 68 | 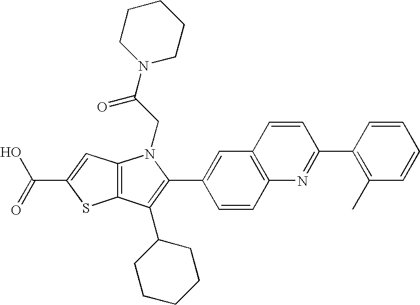 | 6-Cyclohexyl-4-(2-oxo-2-piperidin- 1-yl-ethyl)-5-(2-o-tolyl-quinolin-6- yl)-4H-thieno[3,2-b]pyrrole-2- carboxylic acid |
| 69 | 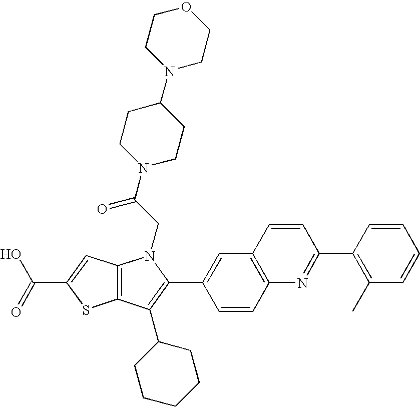 | 6-Cyclohexyl-4-[2-(4-morpholin-4- yl-piperidin-1-yl)-2-oxo-ethyl]-5-(2- o-tolyl-quinolin-6-yl)-4H- thieno[3,2-b]pyrrole-2-carboxylic acid |
| 70 | 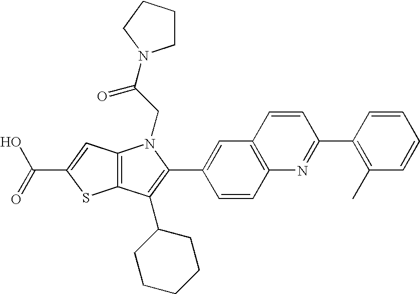 | 6-Cyclohexyl-4-(2-oxo-2- pyrrolidin-1-yl-ethyl)-5-(2-o-tolyl- quinolin-6-yl)-4H-thieno[3,2- b]pyrrole-2-carboxylic acid |
| 71 | 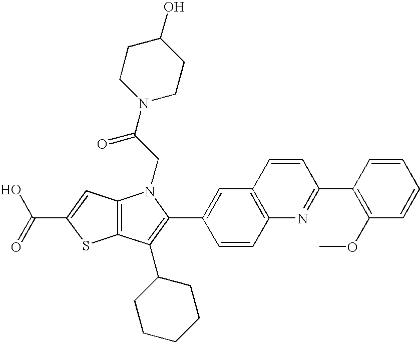 | 6-Cyclohexyl-4-[2-(4-hydroxy- piperidin-1-yl)-2-oxo-ethyl]-5-[2- (2-methoxy-phenyl)-quinolin-6-yl]- 4H-thieno[3,2-b]pyrrole-2- carboxylic acid |
| 72 | 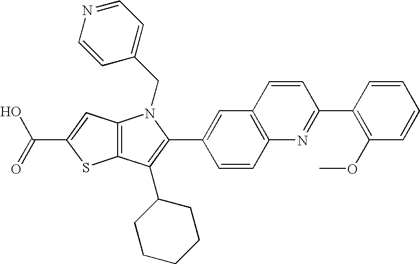 | 6-Cyclohexyl-5-[2-(2-methoxy- phenyl)-quinolin-6-yl]-4-pyridin-4- ylmethyl-4H-thieno[3,2-b]pyrrole- 2-carboxylic acid |
| 73 | 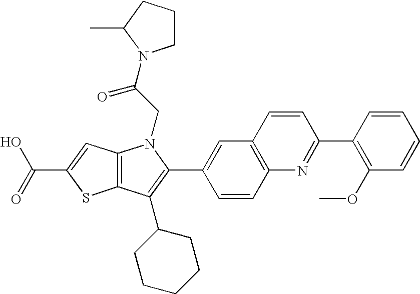 | 6-Cyclohexyl-5-[2-(2-methoxy- phenyl)-quinolin-6-yl]-4-[2-(2- methyl-pyrrolidin-1-yl)-2-oxo- ethyl]-4H-thieno[3,2-b]pyrrole-2- carboxylic acid |
| 74 | 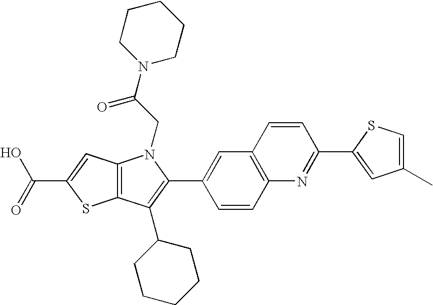 | 6-Cyclohexyl-5-[2-(4-methyl- thiophen-2-yl)-quinolin-6-yl]-4-(2- oxo-2-piperidin-1-yl-ethyl)-4H- thieno[3,2-b]pyrrole-2-carboxylic acid |
| 75 | 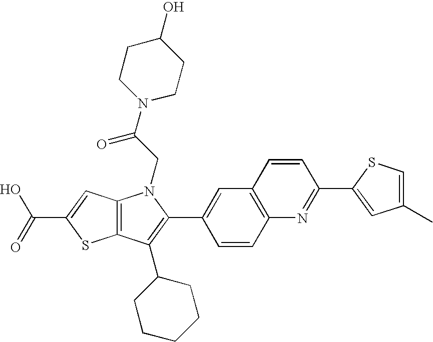 | 6-Cyclohexyl-4-[2-(4-hydroxy- piperidin-1-yl)-2-oxo-ethyl]-5-[2- (4-methyl-thiophen-2-yl)-quinolin- 6-yl]-4H-thieno[3,2-b]pyrrole-2- carboxylic acid |
| 76 | 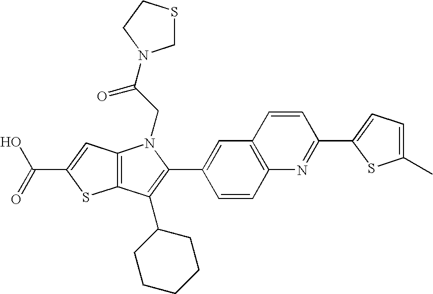 | 6-Cyclohexyl-5-[2-(5-methyl- thiophen-2-yl)-quinolin-6-yl]-4-(2- oxo-2-thiazolidin-3-yl-ethyl)-4H- thieno[3,2-b]pyrrole-2-carboxylic acid |
| 77 | 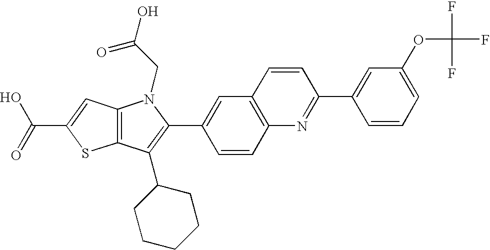 | 4-Carboxymethyl-6-cyclohexyl-5- [2-(3-trifluoromethoxy-phenyl)- quinolin-6-yl]-4H-thieno[3,2- b]pyrrole-2-carboxylic acid |
| 78 | 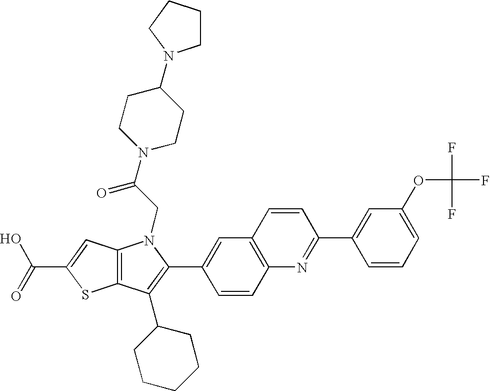 | 6-Cyclohexyl-4-[2-oxo-2-(4- pyrrolidin-1-yl-piperidin-1-yl)- ethyl]-5-[2-(3-trifluoromethoxy- phenyl)-quinolin-6-yl]-4H- thieno[3,2-b]pyrrole-2-carboxylic acid |
| 79 | 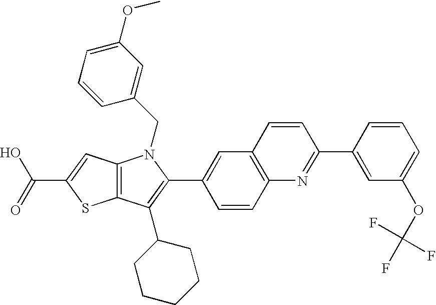 | 6-Cyclohexyl-4-(3-methoxy- benzyl)-5-[2-(3-trifluoromethoxy- phenyl)-quinolin-6-yl]-4H- thieno[3,2-b]pyrrole-2-carboxylic acid |
| 80 | 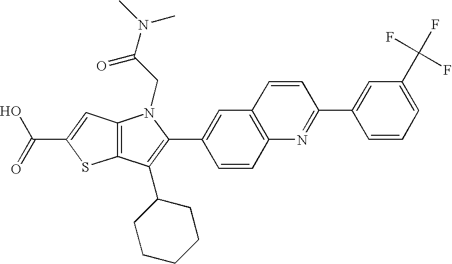 | 6-Cyclohexyl-4- dimethylcarbamoylmethyl-5-[2-(3- trifluoromethyl-phenyl)-quinolin-6- yl]-4H-thieno[3,2-b]pyrrole-2- carboxylic acid |
| 81 | 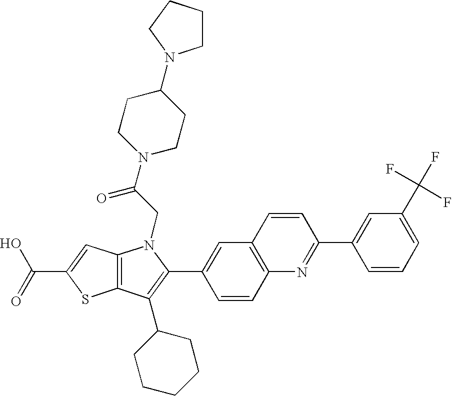 | 6-Cyclohexyl-4-[2-oxo-2-(4- pyrrolidin-1-yl-piperidin-1-yl)- ethyl]-5-[2-(3-trifluoromethyl- phenyl)-quinolin-6-yl]-4H- thieno[3,2-b]pyrrole-2- carboxylic acid |
| 82 | 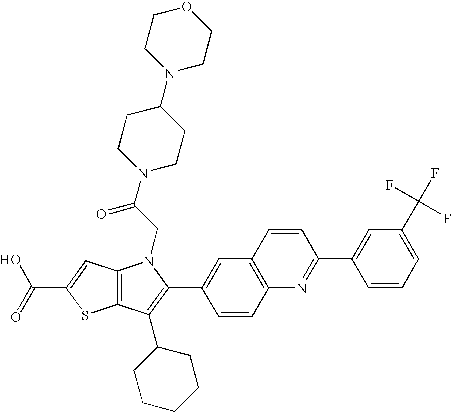 | 6-Cyclohexyl-4-[2-(4-morpholin-4- yl-piperidin-1-yl)-2-oxo-ethyl]-5-[2- (3-trifluoromethyl-phenyl)- quinolin-6-yl]-4H-thieno[3,2- b]pyrrole-2-carboxylic acid |
| 83 | 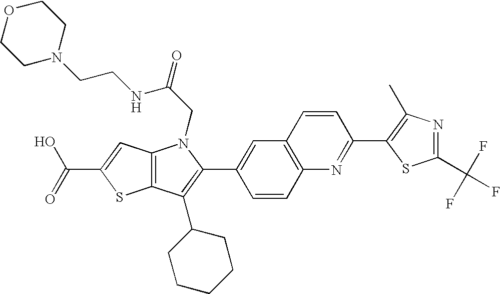 | 6-Cyclohexyl-5-[2-(4-methyl-2- trifluoromethyl-thiazol-5-yl)- quinolin-6-yl]-4-[(2-morpholin-4-yl- ethylcarbamoyl)-methyl]-4H- thieno[3,2-b]pyrrole-2-carboxylic acid |
| 84 | 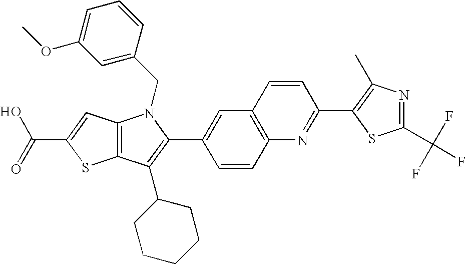 | 6-Cyclohexyl-4-(3-methoxy- benzyl)-5-[2-(4-methyl-2- trifluoromethyl-thiazol-5-yl)- quinolin-6-yl]-4H-thieno[3,2- b]pyrrole-2-carboxylic acid |
| 85 | 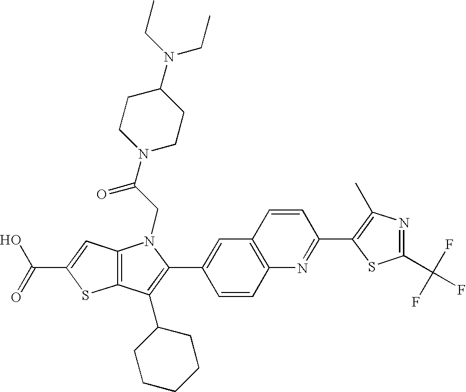 | 6-Cyclohexyl-4-[2-(4- diethylamino-piperidin-1-yl)-2- oxo-ethyl]-5-[2-(4-methyl-2- trifluoromethyl-thiazol-5-yl)- quinolin-6-yl]-4H-thieno[3,2- b]pyrrole-2-carboxylic acid |
| 86 | 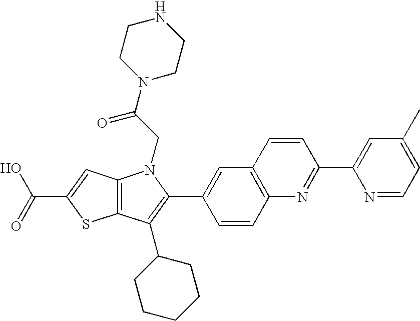 | 6-Cyclohexyl-5-[2-(4-methyl- pyridin-2-yl)-quinolin-6-yl]-4-(2- oxo-2-piperazin-1-yl-ethyl)-4H- thieno[3,2-b]pyrrole-2-carboxylic acid |
| 87 | 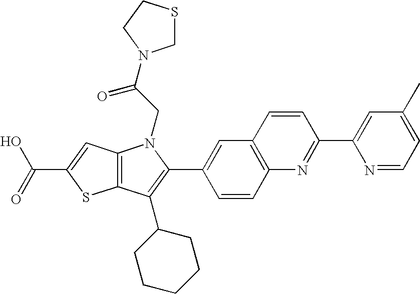 | 6-Cyclohexyl-5-[2-(4-methyl- pyridin-2-yl)-quinolin-6-yl]-4-(2- oxo-2-thiazolidin-3-yl-ethyl)-4H- thieno[3,2-b]pyrrole-2-carboxylic acid |
| 88 | 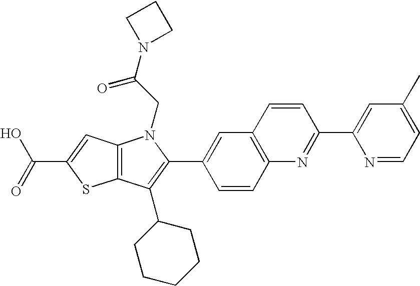 | 4-(2-Azetidin-1-yl-2-oxo-ethyl)-6- cyclohexyl-5-[2-(4-methyl-pyridin- 2-yl)-quinolin-6-yl]-4H-thieno[3,2- b]pyrrole-2-carboxylic acid |
| 89 | 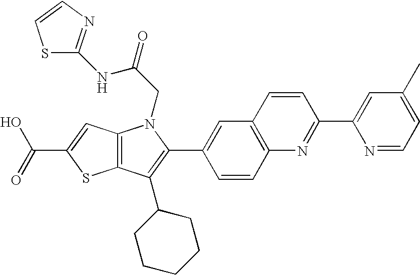 | 6-Cyclohexyl-5-[2-(4-methyl- pyridin-2-yl)-quinolin-6-yl]-4- (thiazol-2-ylcarbamoylmethyl)-4H- thieno[3,2-b]pyrrole-2-carboxylic acid |
| 90 | 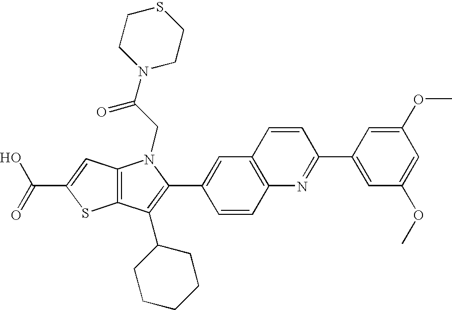 | 6-Cyclohexyl-5-[2-(3,5-dimethoxy- phenyl)-quinolin-6-yl]-4-(2-oxo-2- thiomorpholin-4-yl-ethyl)-4H- thieno[3,2-b]pyrrole-2-carboxylic acid |
| 91 | 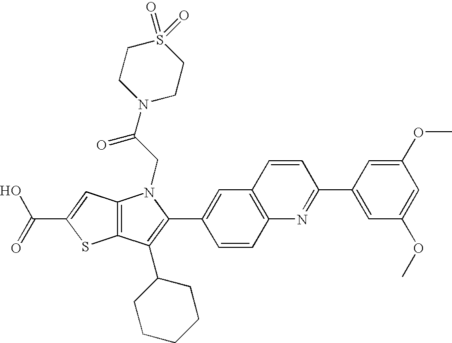 | 6-Cyclohexyl-5-[2-(3,5-dimethoxy- phenyl)-quinolin-6-yl]-4-[2-(1,1- dioxo-1-thiomorpholin-4-yl)-2-oxo- ethyl]-4H-thieno[3,2-b]pyrrole-2- carboxylic acid |
| 92 | 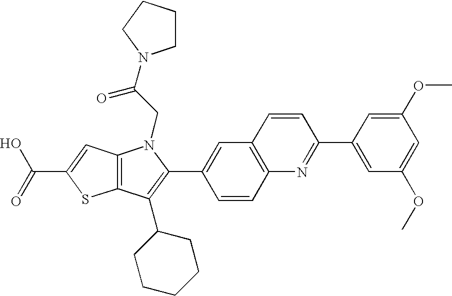 | 6-Cyclohexyl-5-[2-(3,5-dimethoxy- phenyl)-quinolin-6-yl]-4-(2-oxo-2- pyrrolidin-1-yl-ethyl)-4H- thieno[3,2-b]pyrrole-2-carboxylic acid |
| 93 | 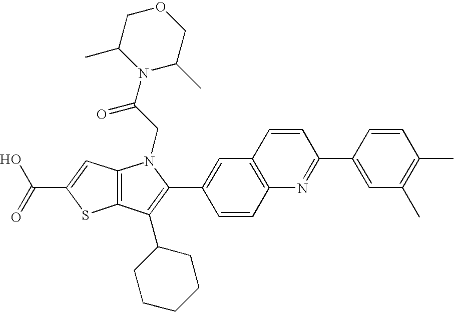 | 6-Cyclohexyl-4-[2-(3,5-dimethyl- morpholin-4-yl)-2-oxo-ethyl]-5-[2- (3,4-dimethyl-phenyl)-quinolin-6- yl]-4H-thieno[3,2-b]pyrrole-2- carboxylic acid |
| 94 | 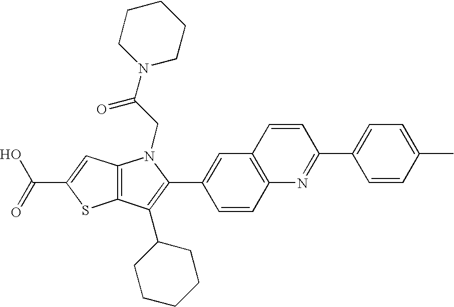 | 6-Cyclohexyl-4-(2-oxo-2-piperidin- 1-yl-ethyl)-5-(2-p-tolyl-quinolin-6- yl)-4H-thieno[3,2-b]pyrrole-2- carboxylic acid |
| 95 | 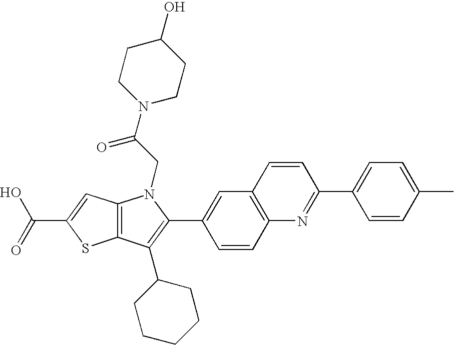 | 6-Cyclohexyl-4-[2-(4-hydroxy- piperidin-1-yl)-2-oxo-ethyl]-5-(2-p- tolyl-quinolin-6-yl)-4H-thieno[3,2- b]pyrrole-2-carboxylic acid |
| 96 | 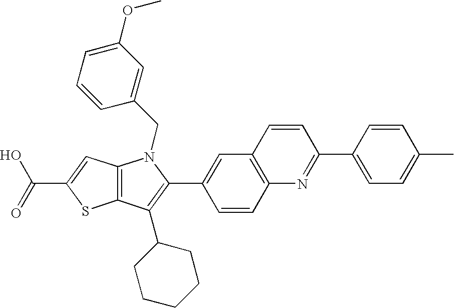 | 6-Cyclohexyl-4-(3-methoxy- benzyl)-5-(2-p-tolyl-quinolin-6-yl)- 4H-thieno[3,2-b]pyrrole-2- carboxylic acid |
| 97 | 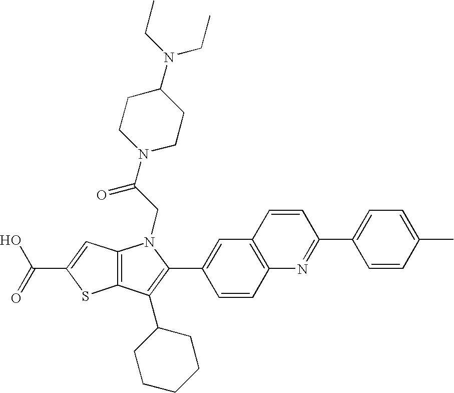 | 6-Cyclohexyl-4-[2-(4- diethylamino-piperidin-1-yl)-2- oxo-ethyl]-5-(2-p-tolyl-quinolin-6- yl)-4H-thieno[3,2-b]pyrrole-2- carboxylic acid |
| 98 | 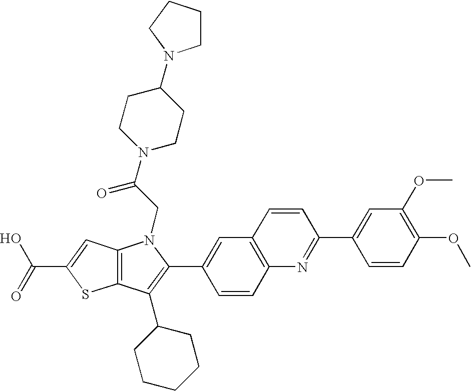 | 6-Cyclohexyl-5-[2-(3,4-dimethoxy- phenyl)-quinolin-6-yl]-4-[2-oxo-2- (4-pyrrolidin-1-yl-piperidin-1-yl)- ethyl]-4H-thieno[3,2-b]pyrrole-2- carboxylic acid |
| 99 | 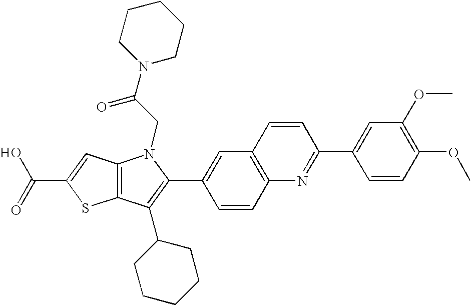 | 6-Cyclohexyl-5-[2-(3,4-dimethoxy- phenyl)-quinolin-6-yl]-4-(2-oxo-2- piperidin-1-yl-ethyl)-4H- thieno[3,2-b]pyrrole-2- carboxylic acid |
| 100 | 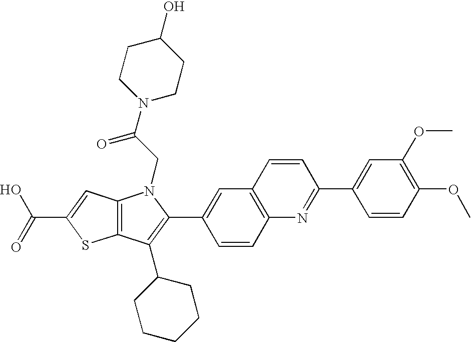 | 6-Cyclohexyl-5-[2-(3,4-dimethoxy- phenyl)-quinolin-6-yl]-4-[2-(4- hydroxy-piperidin-1-yl)-2-oxo- ethyl]-4H-thieno[3,2-b]pyrrole-2- carboxylic acid |
| 101 | 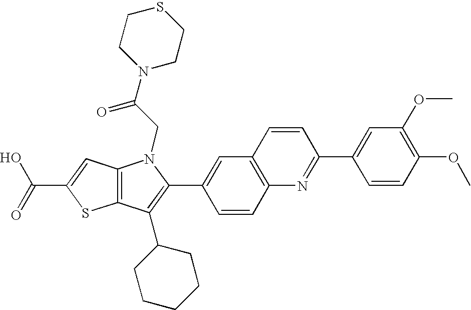 | 6-Cyclohexyl-5-[2-(3,4-dimethoxy- phenyl)-quinolin-6-yl]-4-(2-oxo-2- thiomorpholin-4-yl-ethyl)-4H- thieno[3,2-b]pyrrole-2-carboxylic acid |
| 102 | 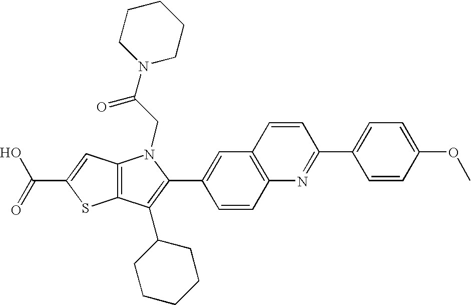 | 6-Cyclohexyl-5-[2-(4-methoxy- phenyl)-quinolin-6-yl]-4-(2-oxo-2- piperidin-1-yl-ethyl)-4H- thieno[3,2-b]pyrrole-2-carboxylic acid |
| 103 | 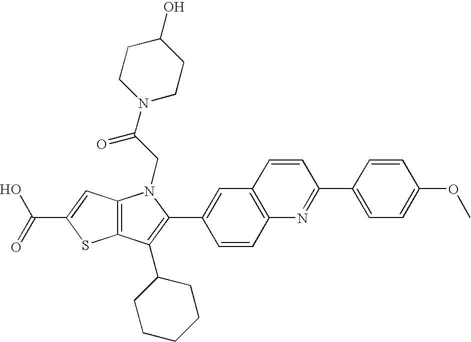 | 6-Cyclohexyl-4-[2-(4-hydroxy- piperidin-1-yl)-2-oxo-ethyl]-5-[2- (4-methoxy-phenyl)-quinolin-6-yl]- 4H-thieno[3,2-b]pyrrole-2- carboxylic acid |
| 104 | 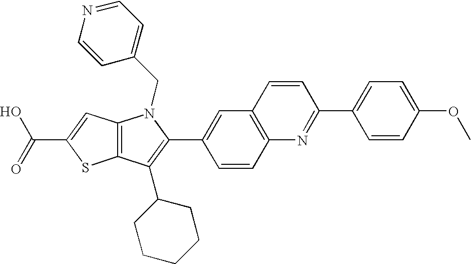 | 6-Cyclohexyl-5-[2-(4-methoxy- phenyl)-quinolin-6-yl]-4-pyridin-4- ylmethyl-4H-thieno[3,2-b]pyrrole- 2-carboxylic acid |
| 105 | 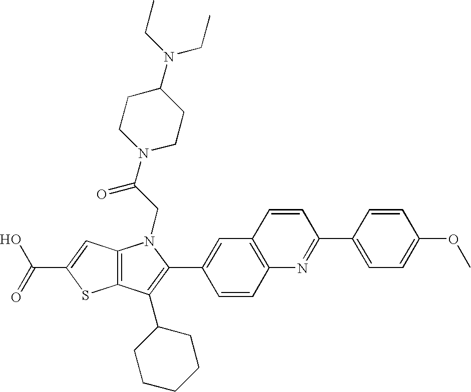 | 6-Cyclohexyl-4-[2-(4- diethylamino-piperidin-1-yl)-2- oxo-ethyl]-5-[2-(4-methoxy- phenyl)-quinolin-6-yl]-4H- thieno[3,2-b]pyrrole-2-carboxylic acid |
| 106 | 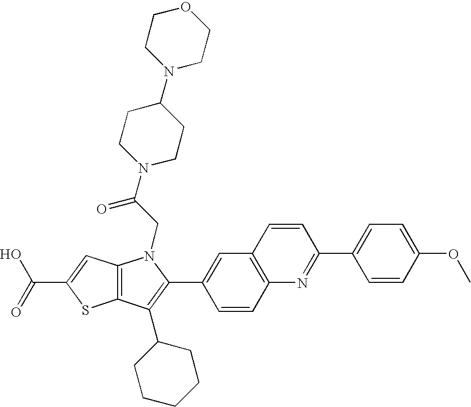 | 6-Cyclohexyl-5-[2-(4-methoxy- phenyl)-quinolin-6-yl]-4-[2-(4- morpholin-4-yl-piperidin-1-yl)-2- oxo-ethyl]-4H-thieno[3,2- b]pyrrole-2-carboxylic acid |
| 107 | 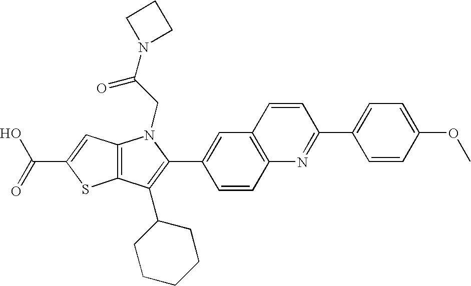 | 4-(2-Azetidin-1-yl-2-oxo-ethyl)-6- cyclohexyl-5-[2-(4-methoxy- phenyl)-quinolin-6-yl]-4H- thieno[3,2-b]pyrrole-2-carboxylic acid |
| 108 | 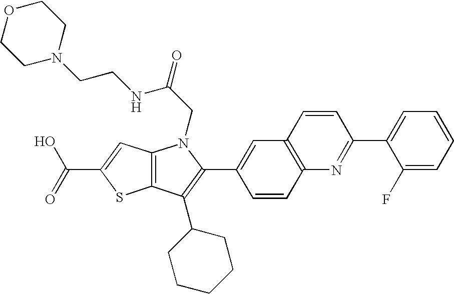 | 6-Cyclohexyl-5-[2-(2-fluoro- phenyl)-quinolin-6-yl]-4-[(2- morpholin-4-yl-ethylcarbamoyl)- methyl]-4H-thieno[3,2-b]pyrrole-2- carboxylic acid |
| 109 | 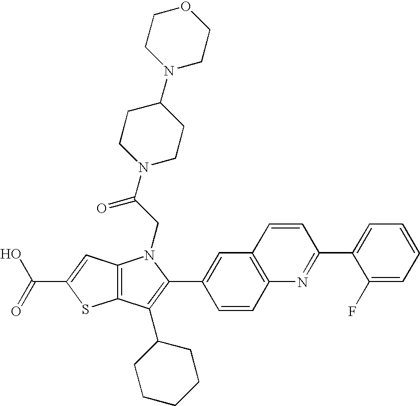 | 6-Cyclohexyl-5-[2-(2-fluoro- phenyl)-quinolin-6-yl]-4-[2-(4- morpholin-4-yl-piperidin-1-yl)-2- oxo-ethyl]-4H-thieno[3,2- b]pyrrole-2-carboxylic acid |
| 110 | 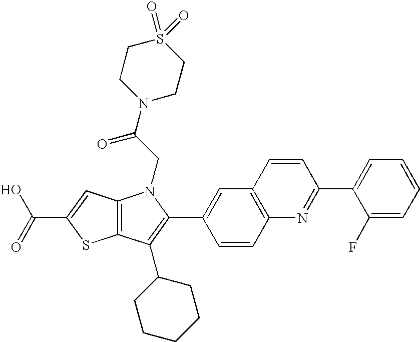 | 6-Cyclohexyl-4-[2-(1,1-dioxo-1- thiomorpholin-4-yl)-2-oxo-ethyl]-5- [2-(2-fluoro-phenyl)-quinolin-6-yl]- 4H-thieno[3,2-b]pyrrole-2- carboxylic acid |
| 111 | 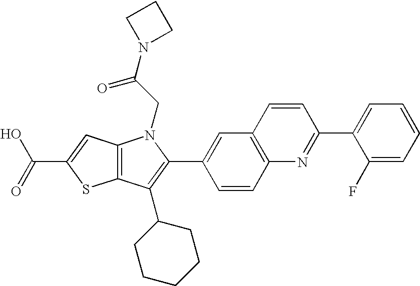 | 4-(2-Azetidin-1-yl-2-oxo-ethyl)-6- cyclohexyl-5-[2-(2-fluoro-phenyl)- quinolin-6-yl]-4H-thieno[3,2- b]pyrrole-2- carboxylic acid |
| 112 | 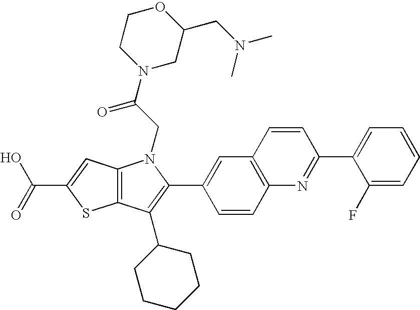 | 6-Cyclohexyl-4-[2-(2- dimethylaminomethyl-morpholin- 4-yl)-2-oxo-ethyl]-5-[2-(2-fluoro- phenyl)-quinolin-6-yl]-4H- thieno[3,2-b]pyrrole-2- carboxylic acid |
| 113 | 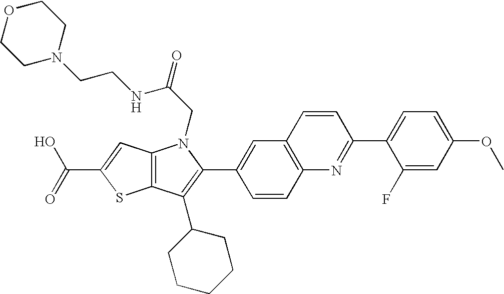 | 6-Cyclohexyl-5-[2-(2-fluoro-4- methoxy-phenyl)-quinolin-6-yl]-4- [(2-morpholin-4-yl- ethylcarbamoyl)-methyl]-4H- thieno[3,2-b]pyrrole-2- carboxylic acid |
| 114 | 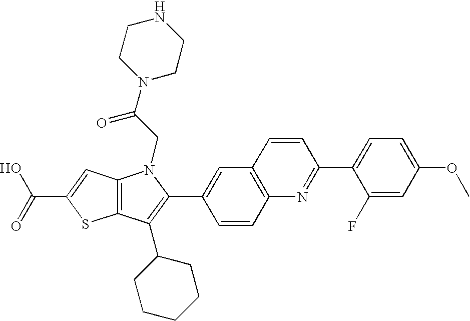 | 6-Cyclohexyl-5-[2-(2-fluoro-4- methoxy-phenyl)-quinolin-6-yl]-4- (2-oxo-2-piperazin-1-yl-ethyl)-4H- thieno[3,2-b]pyrrole-2- carboxylic acid |
| 115 | 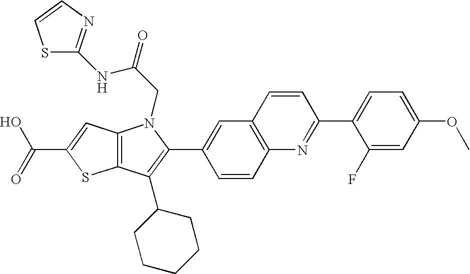 | 6-Cyclohexyl-5-[2-(2-fluoro-4- methoxy-phenyl)-quinolin-6-yl]-4- (thiazol-2-ylcarbamoylmethyl)-4H- thieno[3,2-b]pyrrole-2-carboxylic acid |
| 116 | 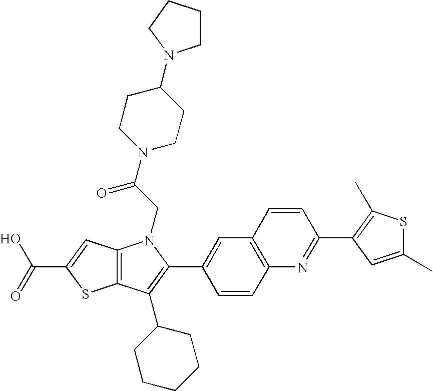 | 6-Cyclohexyl-5-[2-(2,5-dimethyl- thiophen-3-yl)-quinolin-6-yl]-4-[2- oxo-2-(4-pyrrolidin-1-yl-piperidin- 1-yl)-ethyl]-4H-thieno[3,2- b]pyrrole-2-carboxylic acid |
| 117 | 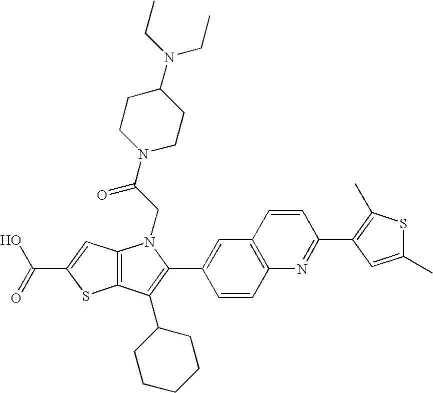 | 6-Cyclohexyl-4-[2-(4- diethylamino-piperidin-1-yl)-2- oxo-ethyl]-5-[2-(2,5-dimethyl- thiophen-3-yl)-quinolin-6-yl]-4H- thieno[3,2-b]pyrrole-2-carboxylic acid |
| 118 | 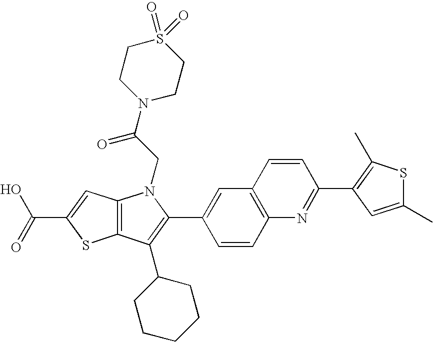 | 6-Cyclohexyl-5-[2-(2,5-dimethyl- thiophen-3-yl)-quinolin-6-yl]-4-[2- (1,1-dioxo-1-thiomorpholin-4-yl)- 2-oxo-ethyl]-4H-thieno[3,2- b]pyrrole-2-carboxylic acid |
| 119 | 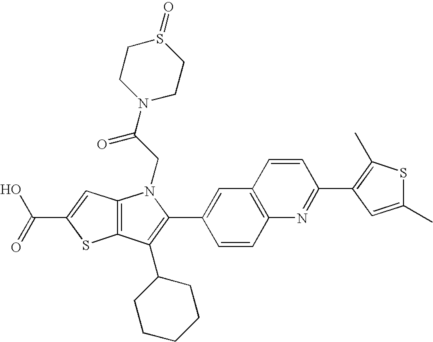 | 6-Cyclohexyl-5-[2-(2,5-dimethyl- thiophen-3-yl)-quinolin-6-yl]-4-[2- oxo-2-(1-oxo-1-thiomorpholin-4- yl)-ethyl-4H-thieno[3,2-b]pyrrole- 2-carboxylic acid |
| 120 | 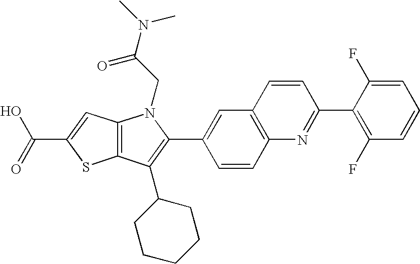 | 6-Cyclohexyl-5-[2-(2,6-difluoro- phenyl)-quinolin-6-yl]-4- dimethylcarbamoylmethyl-4H- thieno[3,2-b]pyrrole-2-carboxylic acid |
| 121 | 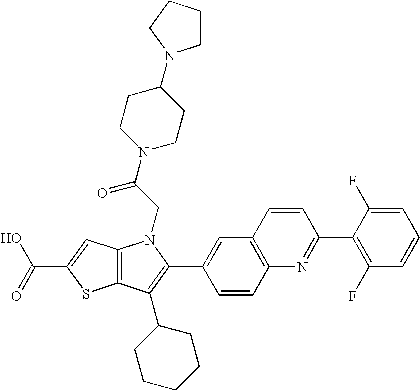 | 6-Cyclohexyl-5-[2-(2,6-difluro- phenyl)-quinolin-6-yl]-4-[2-oxo-2- (4-pyrrolidin-1-yl-piperidin-1-yl)- ethyl]-4H-thieno[3,2-b]pyrrole-2- carboxylic acid |
| 122 | 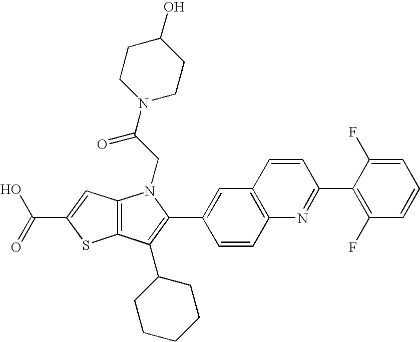 | 6-Cyclohexyl-5-[2-(2,6-difluoro- phenyl)-quinolin-6-yl]-4-[2-(4- hydroxy-piperidin-1-yl)-2-oxo- ethyl]-4H-thieno[3,2-b]pyrrole-2- carboxylic acid |
| 123 | 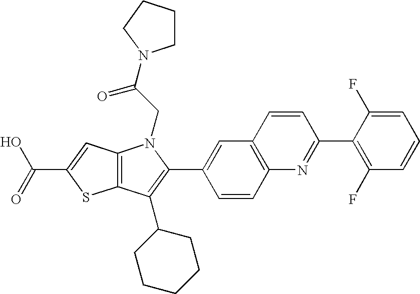 | 6-Cyclohexyl-5-[2-(2,6-difluoro- phenyl)-quinolin-6-yl]-4-[2-oxo-2- pyrrolidin-1-yl-ethyl)-4H- thieno[3,2-b]pyrrole-2-carboxylic acid |
| 124 | 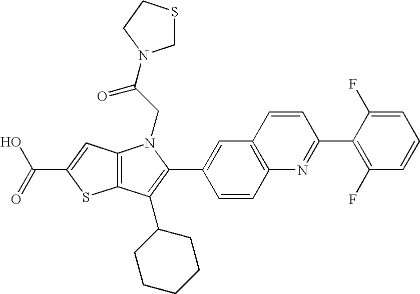 | 6-Cyclohexyl-5-[2-(2,6-difluoro- phenyl)-quinolin-6-yl]-4-(2-oxo-2- thiazolidin-3-yl-ethyl)-4H- thieno[3,2-b]pyrrole-2-carboxylic acid |
| 125 | 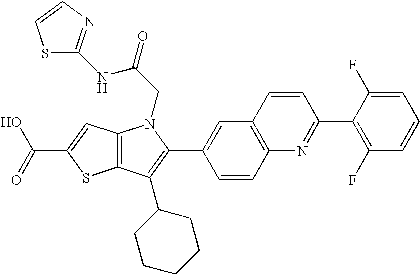 | 6-Cyclohexyl-5-[2-(2,6-difluoro- phenyl)-quinolin-6-yl]-4-(thiazol-2- ylcarbamoylmethyl)-4H- thieno[3,2-b]pyrrole-2-carboxylic acid |
| 126 | 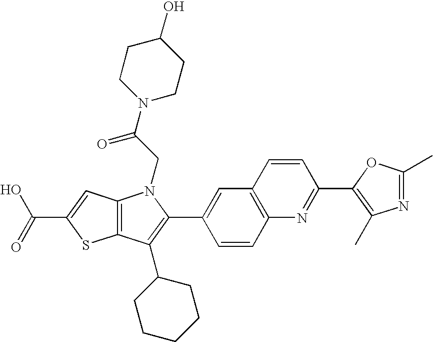 | 6-Cyclohexyl-5-[2-(2,4-dimethyl- oxazol-5-yl)-quinolin-6-yl]-4-[2-(4- hydroxy-piperidin-1-yl)-2-oxo- ethyl]-4H-thieno[3,2-b]pyrrole-2- carboxylic acid |
| 127 | 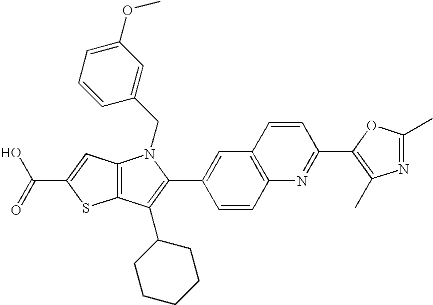 | 6-Cyclohexyl-5-[2-(2,4-dimethyl- oxazol-5-yl)-quinolin-6-yl]-4-(3- methoxy-benzyl)-4H-thieno[3,2- b]pyrrole-2-carboxylic acid |
| 128 | 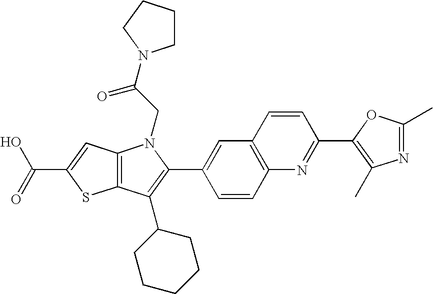 | 6-Cyclohexyl-5-[2-(2,4-dimethyl- oxazol-5-yl)-quinolin-6-yl]-4-(2- oxo-2-pyrrolidin-1-yl-ethyl)-4H- thieno[3,2-b]pyrrole-2-carboxylic acid |
| 129 | 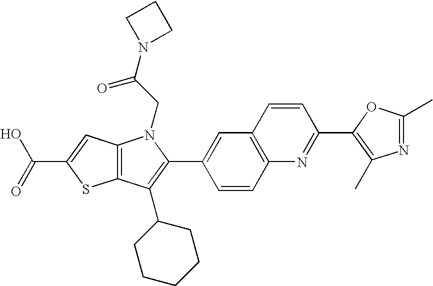 | 4-(2-Azetidin-1-yl-2-oxo-ethyl)-6- cyclohexyl-5-[2-(2,4-dimethyl- oxazol-5-yl)-quinolin-6-yl]-4H- thieno[3,2-b]pyrrole-2-carboxylic acid |
| 130 | 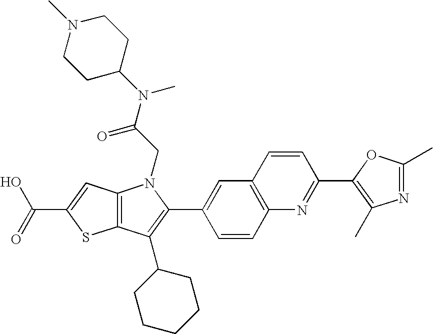 | 6-Cyclohexyl-5-[2-(2,4-dimethyl- oxazol-5-yl)-quinolin-6-yl]-4- {[methyl-(1-methyl-piperidin-4-yl)- carbamoyl]-methyl}-4H- thieno[3,2-b]pyrrole-2-carboxylic acid |
| 131 | 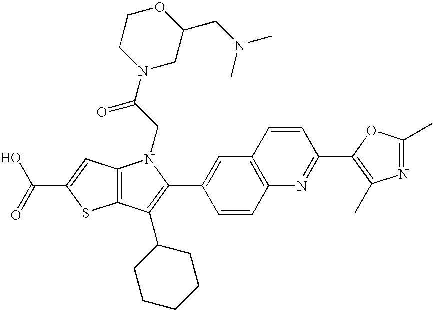 | 6-Cyclohexyl-4-[2-(2- dimethylaminomethyl-morpholin- 4-yl)-2-oxo-ethyl]-5-[2-(2,4- dimethyl-oxazol-5-yl)-quinolin-6- yl]-4H-thieno[3,2-b]pyrrole-2- carboxylic acid |
| 132 | 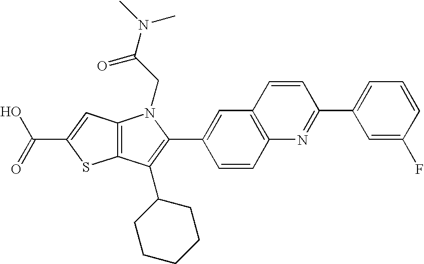 | 6-Cyclohexyl-4- dimethylcarbamoylmethyl-5-[2-(3- fluoro-phenyl)-quinolin-6-yl]-4H- thieno[3,2-b]pyrrole-2-carboxylic acid |
| 133 | 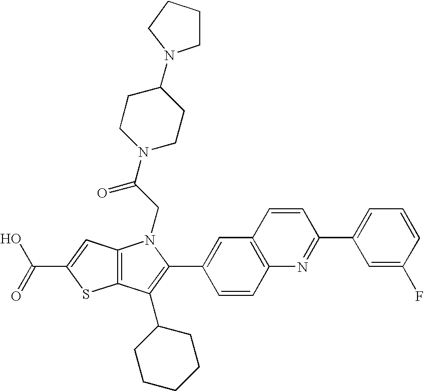 | 6-Cyclohexyl-5-[2-(3-fluoro- phenyl)-quinolin-6-yl]-4-[2-oxo-2- (4-pyrrolidin-1-yl-piperidin-1-yl)- ethyl]-4H-thieno[3,2-b]pyrrole-2- carboxylic acid |
| 134 | 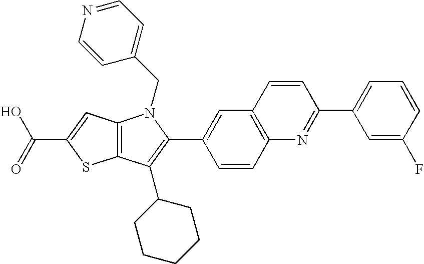 | 6-Cyclohexyl-5-[2-(3-fluoro- phenyl)-quinolin-6-yl]-4-pyridin-4- ylmethyl-4H-thieno[3,2-b]pyrrole- 2-carboxylic acid |
| 135 | 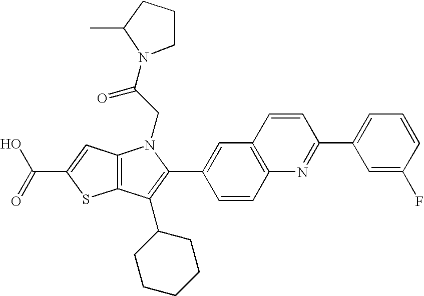 | 6-Cyclohexyl-5-[2-(3-fluoro- phenyl)-quinolin-6-yl]-4-[2-(2- methyl-pyrrolidin-1-yl)-2-oxo- ethyl]-4H-thieno[3,2-b]pyrrole-2- carboxylic acid |
| 136 | 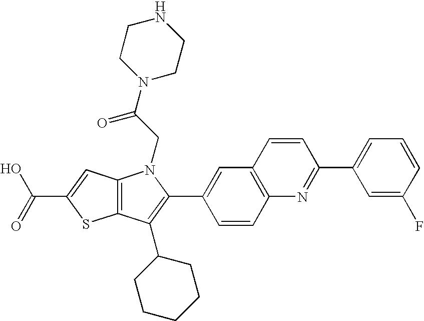 | 6-Cyclohexyl-5-[2-(3-fluoro- phenyl)-quinolin-6-yl]-4-(2-oxo-2- piperazin-1-yl-ethyl)-4H- thieno[3,2-b]pyrrole-2-carboxylic acid |
| 137 | 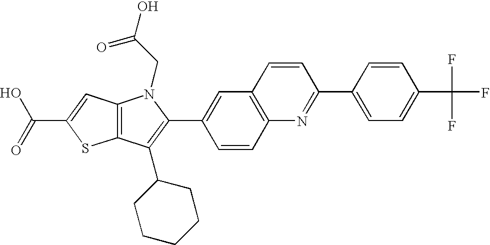 | 4-Carboxymethyl-6-cyclohexyl-5- [2-(4-trifluoromethyl-phenyl)- quinolin-6-yl]-4H-thieno[3,2- b]pyrrole-2-carboxylic acid |
| 138 | 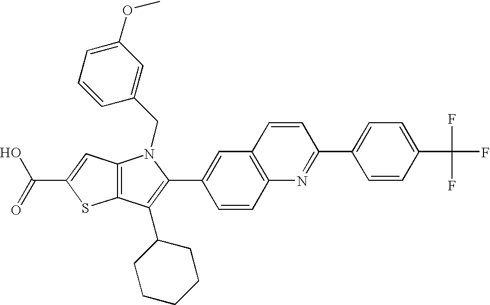 | 6-Cyclohexyl-4-(3-methoxy- benzyl)-5-[2-(4-trifluoromethyl- phenyl)-quinolin-6-yl]-4H- thieno[3,2-b]pyrrole-2-carboxylic acid |
| 139 | 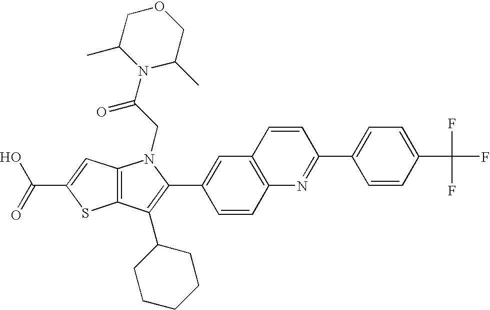 | 6-Cyclohexyl-4-[2-(3,5-dimethyl- morpholin-4-yl)-2-oxo-ethyl]-5-[2- (4-trifluoromethyl-phenyl)- quinolin-6-yl]-4H-thieno[3,2- b]pyrrole-2-carboxylic acid |
| 140 | 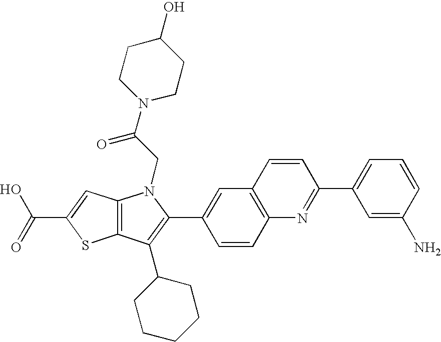 | 5-[2-(3-Amino-phenyl)-quinolin-6- yl]-6-cyclohexyl-4-[2-(4-hydroxy- piperidin-1-yl)-2-oxo-ethyl]-4H- thieno[3,2-b]pyrrole-2-carboxylic acid |
| 141 | 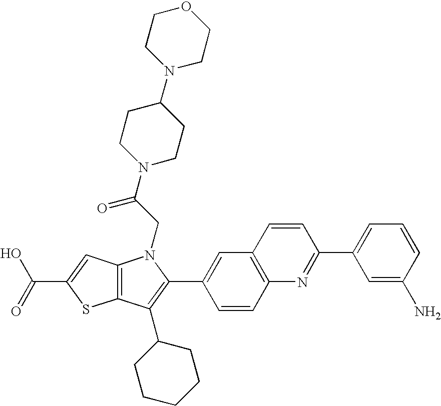 | 5-[2-(3-Amino-phenyl)-quinolin-6- yl]-6-cyclohexyl-4-[2-(4- morpholin-4-yl-piperidin-1-yl)-2- oxo-ethyl]-4H-thieno[3,2- b]pyrrole-2-carboxylic acid |
| 142 | 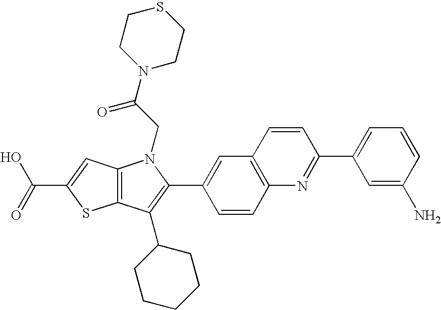 | 5-[2-(3-Amino-phenyl)-quinolin-6- yl]-6-cyclohexyl-4-(2-oxo-2- thiomorpholin-4-yl-ethyl)-4H- thieno[3,2-b]pyrrole-2-carboxylic acid |
| 143 | 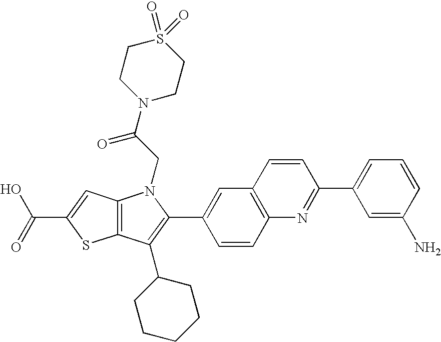 | 5-[2-(3-Amino-phenyl)-quinolin-6- yl]-6-cyclohexyl-4-[2-(1,1-dioxo-1- thiomorpholin-4-yl)-2-oxo-ethyl]- 4H-thieno[3,2-b]pyrrole-2- carboxylic acid |
| 144 | 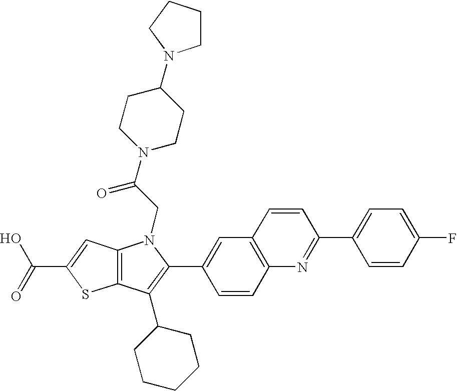 | 6-Cyclohexyl-5-[2-(4-fluoro- phenyl)-quinolin-6-yl]-4-[2-oxo-2- (4-pyrrolidin-1-yl-piperidin-1-yl)- ethyl]-4H-thieno[3,2-b]pyrrole-2- carboxylic acid |
| 145 | 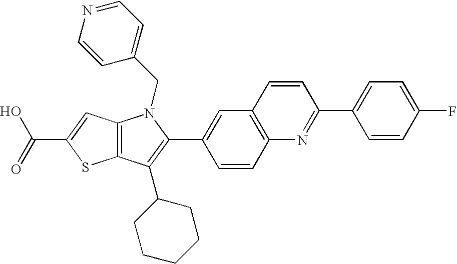 | 6-Cyclohexyl-5-[2-(4-fluoro- phenyl)-quinolin-6-yl]-4-pyridin-4- ylmethyl-4H-thieno[3,2-b]pyrrole- 2-carboxylic acid |
| 146 | 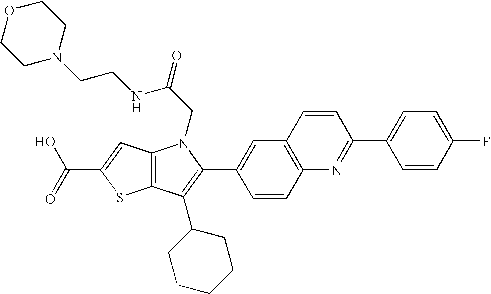 | 6-Cyclohexyl-5-[2-(4-fluoro- phenyl)-quinolin-6-yl]-4-[(2- morpholin-4-yl-ethylcarbamoyl)- methyl]-4H-thieno[3,2-b]pyrrole-2- carboxylic acid |
| 147 | 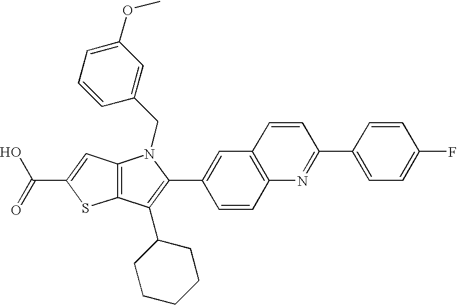 | 6-Cyclohexyl-5-[2-(4-fluoro- phenyl)-quinolin-6-yl]-4-(3- methoxy-benzyl)-4H-thieno[3,2- b]pyrrole-2-carboxylic acid |
| 148 | 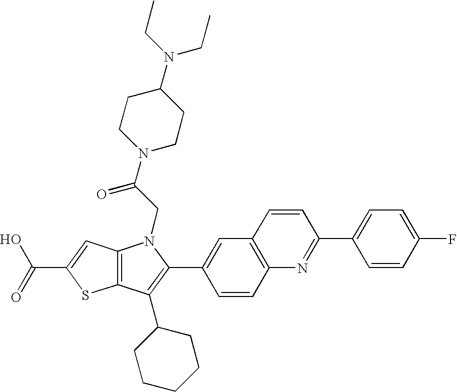 | 6-Cyclohexyl-4-[2-(4- diethylamino-piperidin-1-yl)-2- oxo-ethyl]-5-[2-(4-fluoro-phenyl)- quinolin-6-yl]-4H-thieno[3,2- b]pyrrole-2-carboxylic acid |
| 149 | 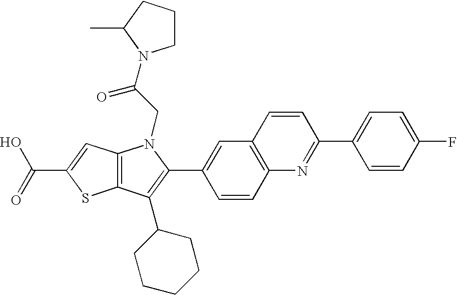 | 6-Cyclohexyl-5-[2-(4-fluoro- phenyl)-quinolin-6-yl]-4-[2-(2- methyl-pyrrolidin-1-yl)-2-oxo- ethyl]-4H-thieno[3,2-b]pyrrole-2- carboxylic acid |
| 150 | 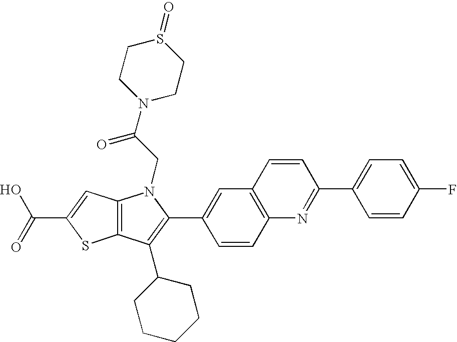 | 6-Cyclohexyl-5-[2-(4-fluoro- phenyl)-quinolin-6-yl]-4-[2-oxo-2- (1-oxo-thiomorpholin-4-yl)-ethyl]- 4H-thieno[3,2-b]pyrrole-2- carboxylic acid |
| 151 | 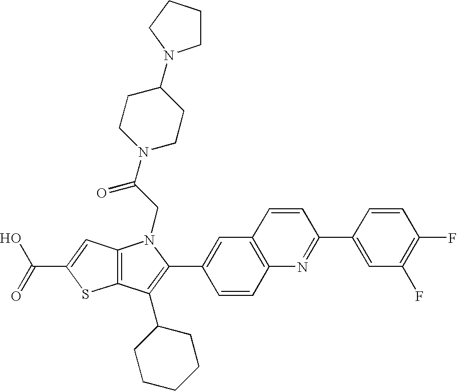 | 6-Cyclohexyl-5-[2-(3,4-difluoro- phenyl)-quinolin-6-yl]-4-[2-oxo-2- (4-pyrrolidin-1-yl-piperidin-1-yl)- ethyl]-4H-thieno[3,2-b]pyrrole-2- carboxylic acid |
| 152 | 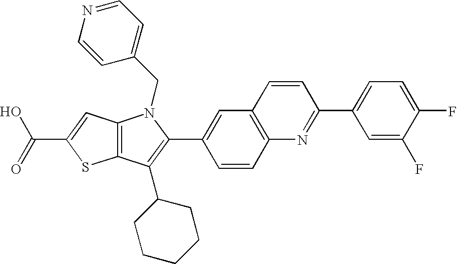 | 6-Cyclohexyl-5-[2-(3,4-difluoro- phenyl)-quinolin-6-yl]-4-pyridin-4- ylmethyl-4H-thieno[3,2-b]pyrrole- 2-carboxylic acid |
| 153 | 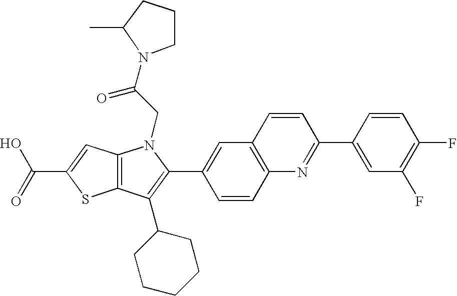 | 6-Cyclohexyl-5-[2-(3,4-difluoro- phenyl)-quinolin-6-yl]-4-[2-(2- methyl-pyrrolidin-1-yl)-2-oxo- ethyl]-4H-thieno[3,2-b]pyrrole-2- carboxylic acid |
| 154 | 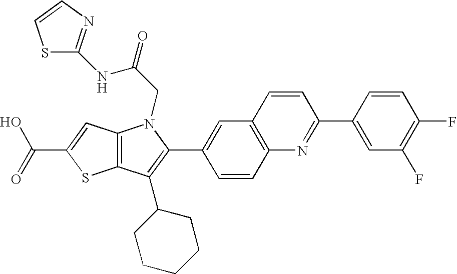 | 6-Cyclohexyl-5-[2-(3,4-difluoro- phenyl)-quinolin-6-yl]-4-(thiazol-2- ylcarbamoylmethyl)-4H- thieno[3,2-b]pyrrole-2-carboxylic acid |
| 155 | 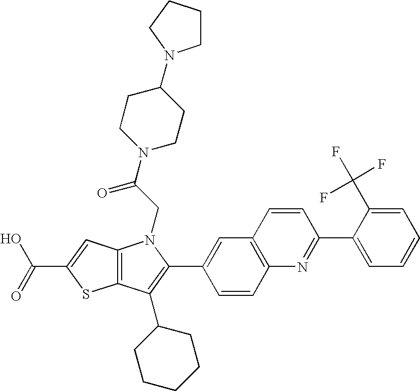 | 6-Cyclohexyl-4-[2-oxo-2-(4- pyrrolidin-1-yl-piperidin-1-yl)- ethyl]-5-[2-(2-trifluoromethyl- phenyl)-quinolin-6-yl]-4H- thieno[3,2-b]pyrrole-2-carboxylic acid |
| 156 | 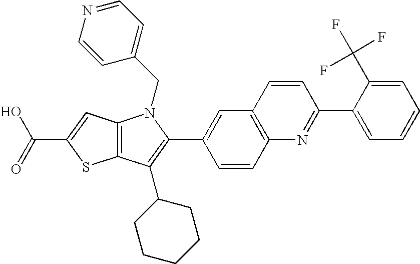 | 6-Cyclohexyl-4-pyridin-4-ylmethyl- 5-[2-(2-trifluoromethyl-phenyl)- quinolin-6-yl]-4H-thieno[3,2- b]pyrrole-2-carboxylic acid |
| 157 | 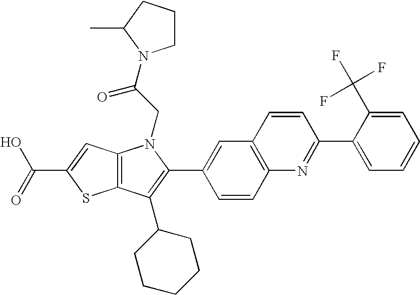 | 6-Cyclohexyl-4-[2-(2-methyl- pyrrolidin-1-yl)-2-oxo-ethyl]-5-[2- (2-trifluoromethyl-phenyl)- quinolin-6-yl]-4H-thieno[3,2- b]pyrrole-2-carboxylic acid |
| 158 | 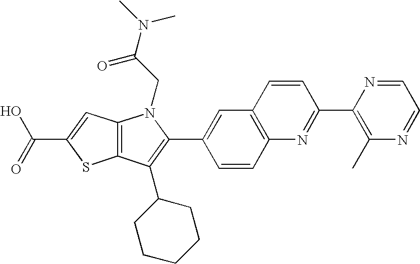 | 6-Cyclohexyl-4- dimethylcarbamoylmethyl-5-[2-(3- methyl-pyrazin-2-yl)-quinolin-6-yl]- 4H-thieno[3,2-b]pyrrole-2- carboxylic acid |
| 159 | 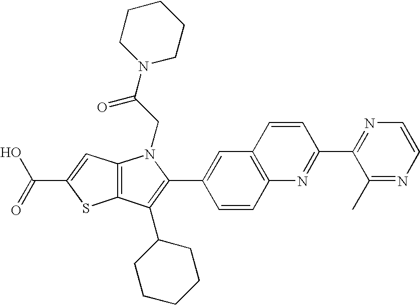 | 6-Cyclohexyl-5-[2-(3-methyl- pyrazin-2-yl)-quinolin-6-yl]-4-(2- oxo-2-piperidin-1-yl-ethyl)-4H- thieno[3,2-b]pyrrole-2-carboxylic acid |
| 160 | 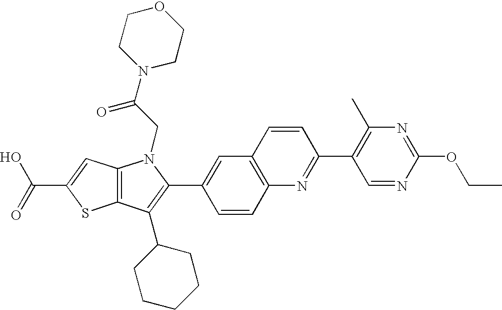 | 6-Cyclohexyl-5-[2-(2-ethoxy-4- methyl-pyrimidin-5-yl)-quinolin-6- yl]-4-(2-morpholin-4-yl-2-oxo- ethyl)-4H-thieno[3,2-b]pyrrole-2- carboxylic acid |
| 161 | 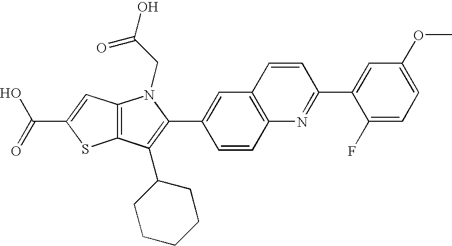 | 4-Carboxymethyl-6-cyclohexyl-5- [2-(2-fluoro-5-methoxy-phenyl)- quinolin-6-yl]-4H-thieno[3,2- b]pyrrole-2-carboxylic acid |
| 162 | 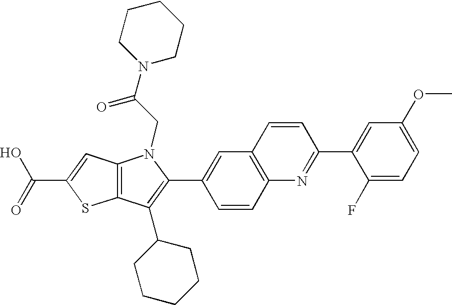 | 6-Cyclohexyl-5-[2-(2-fluoro-5- methoxy-phenyl)-quinolin-6-yl]-4- (2-oxo-2-piperidin-1-yl-ethyl)-4H- thieno[3,2-b]pyrrole-2-carboxylic acid |
| 163 | 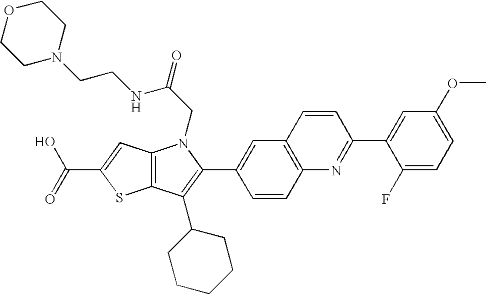 | 6-Cyclohexyl-5-[2-(2-fluoro-5- methoxy-phenyl)-quinolin-6-yl]-4- [(2-morpholin-4-yl- ethylcarbamoyl)-methyl]-4H- thieno[3,2-b]pyrrole-2-carboxylic acid |
| 164 | 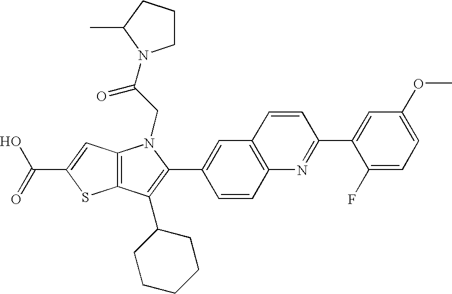 | 6-Cyclohexyl-5-[2-(2-fluoro-5- methoxy-phenyl)-quinolin-6-yl]-4- [2-(2-methyl-pyrrolidin-1-yl)-2- oxo-ethyl]-4H-thieno[3,2- b]pyrrole-2-carboxylic acid |
| 165 | 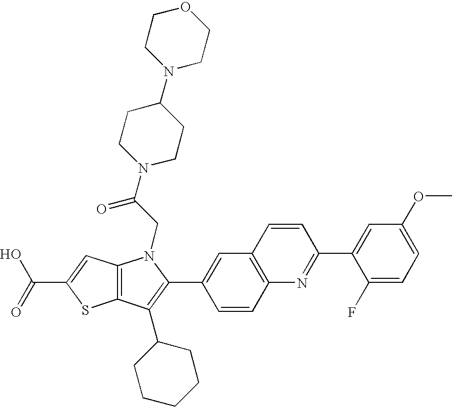 | 6-Cyclohexyl-5-[2-(2-fluoro-5- methoxy-phenyl)-quinolin-6-yl]-4- [2-(4-morpholin-4-yl-piperidin-1- yl)-2-oxo-ethyl]-4H-thieno[3,2- b]pyrrole-2-carboxylic acid |
| 166 | 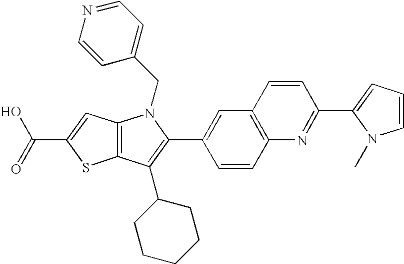 | 6-Cyclohexyl-5-[2-(1-methyl-1H- pyrrol-2-yl)-quinolin-6-yl]-4- pyridin-4-ylmethyl-4H-thieno[3,2- b]pyrrole-2-carboxylic acid |
| 167 | 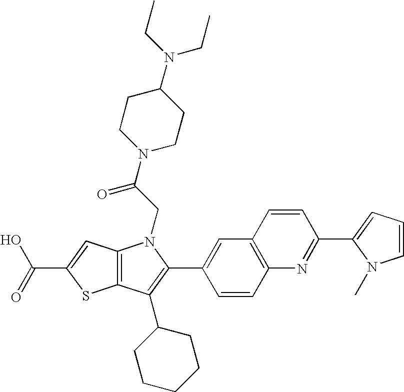 | 6-Cyclohexyl-4-[2-(4- diethylamino-piperidin-1-yl)-2- oxo-ethyl]-5-[2-(1-methyl-1H- pyrrol-2-yl)-quinolin-6-yl]-4H- thieno[3,2-b]pyrrole-2-carboxylic acid |
| 168 | 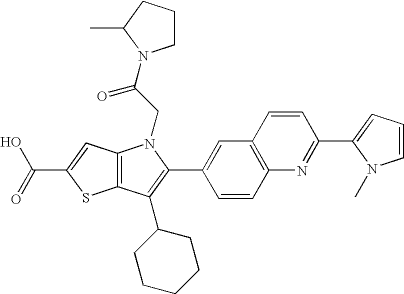 | 6-Cyclohexyl-4-[2-(2-methyl- pyrrolidin-1-yl)-2-oxo-ethyl]-5-[2- (1-methyl-1H-pyrrol-2-yl)-quinolin- 6-yl]-4H-thieno[3,2-b]pyrrole-2- carboxylic acid |
| 169 | 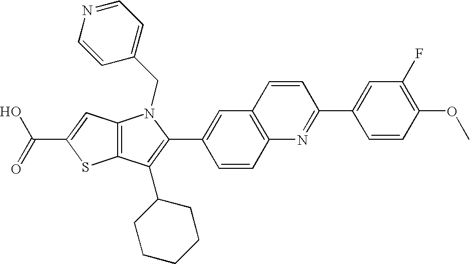 | 6-Cyclohexyl-5-[2-(3-fluoro-4- methoxy-phenyl)-quinolin-6-yl]-4- pyridin-4-ylmethyl-4H-thieno[3,2- b]pyrrole-2-carboxylic acid |
| 170 | 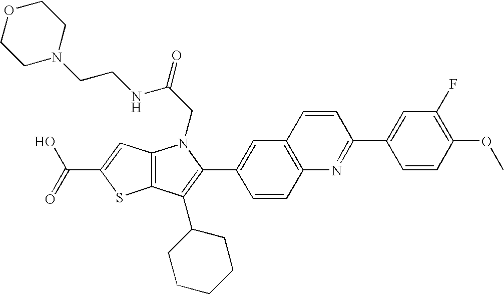 | 6-Cyclohexyl-5-[2-(3-fluoro-4- methoxy-phenyl)-quinolin-6-yl]-4- [(2-morpholin-4-yl- ethylcarbamoyl)-methyl]-4H- thieno[3,2-b]pyrrole-2-carboxylic acid |
| 171 | 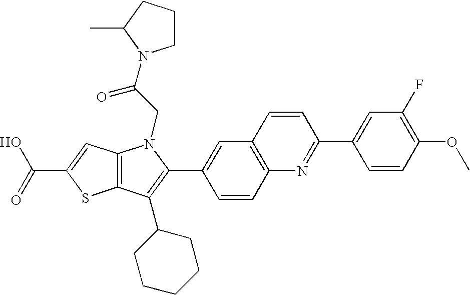 | 6-Cyclohexyl-5-[2-(3-fluoro-4- methoxy-phenyl)-quinolin-6-yl]-4- [2-(2-methyl-pyrrolidin-1-yl)-2- oxo-ethyl]-4H-thieno[3,2- b]pyrrole-2-carboxylic acid |
| 172 | 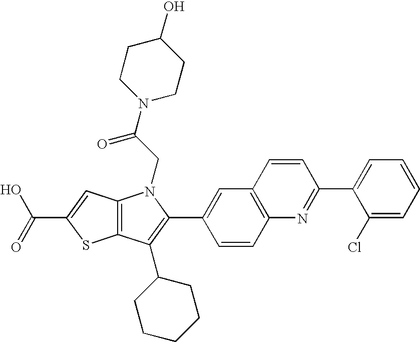 | 5-[2-(2-Chloro-phenyl)-quinolin-6- yl]-6-cyclohexyl-4-[2-(4-hydroxy- piperidin-1-yl)-2-oxo-ethyl]-4H- thieno[3,2-b]pyrrole-2-carboxylic acid |
| 173 | 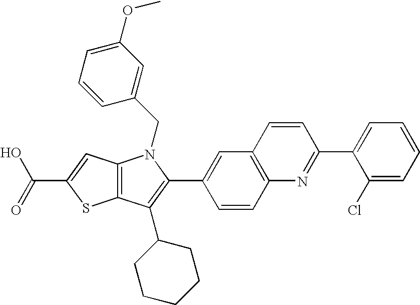 | 5-[2-(2-Chloro-phenyl)-quinolin-6- yl]-6-cyclohexyl-4-(3-methoxy- benzyl)-4H-thieno[3,2-b]pyrrole-2- carboxylic acid |
| 174 | 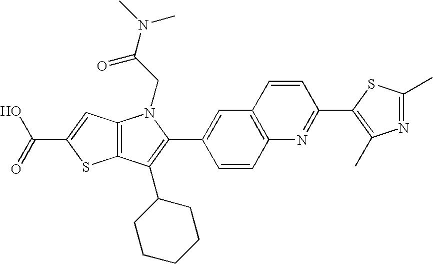 | 6-Cyclohexyl-4- dimethylcarbamoylmethyl-5-[2- (2,4-dimethyl-thiazol-5-yl)- quinolin-6-yl]-4H-thieno[3,2- b]pyrrole-2-carboxylic acid |
| 175 | 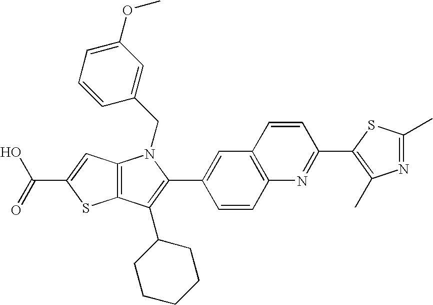 | 6-Cyclohexyl-5-[2-(2,4-dimethyl- thiazol-5-yl)-quinolin-6-yl]-4-(3- methoxy-benzyl)-4H-thieno[3,2- b]pyrrole-2-carboxylic acid |
| 176 | 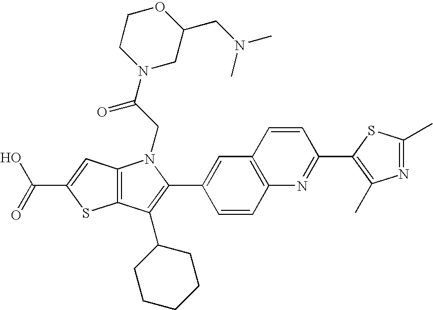 | 6-Cyclohexyl-4-[2-(2- dimethylaminomethyl-morpholin- 4-yl)-2-oxo-ethyl]-5-[2-(2,4- dimethyl-thiazol-5-yl)-quinolin-6- yl]-4H-thieno[3,2-b]pyrrole-2- carboxylic acid |
| 177 | 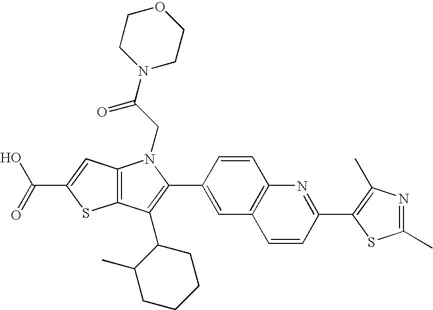 | 5-[2-(2,4-Dimethyl-thiazol-5-yl)- quinolin-6-yl]-6-(2-methyl- cyclohexyl)-4-(2-morpholin-4-yl-2- oxo-ethyl)-4H-thieno[3,2- b]pyrrole-2-carboxylic acid |
| 178 | 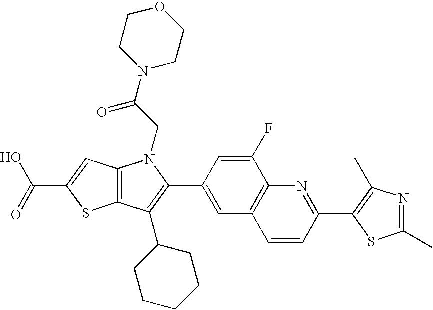 | 6-Cyclohexyl-5-[2-(2,4-dimethyl- thiazol-5-yl)-8-fluoro-quinolin-6- yl]-4-(2-morpholin-4-yl-2-oxo- ethyl)-4H-thieno[3,2-b]pyrrole-2- carboxylic acid |
| 179 | 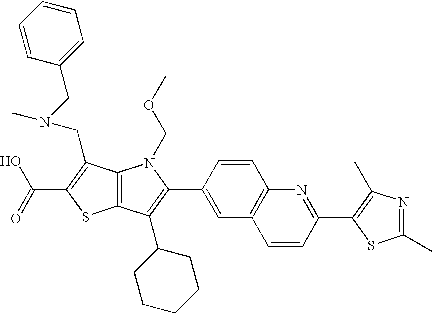 | 3-[(Benzyl-methyl-amino)-methyl]- 6-cyclohexyl-5-[2-(2,4-dimethyl- thiazol-5-yl)-quinolin-6-yl]-4- methoxymethyl-4H-thieno[3,2- b]pyrrole-2-carboxylic acid |
| 180 | 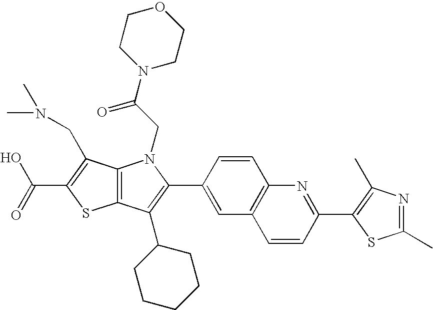 | 6-Cyclohexyl-3- dimethylaminomethyl-5-[2-(2,4- dimethyl-thiazol-5-yl)-quinolin-6- yl]-4-(2-morpholin-4-yl-2-oxo- ethyl)-4H-thieno[3,2-b]pyrrole-2- carboxylic acid |
| 181 | 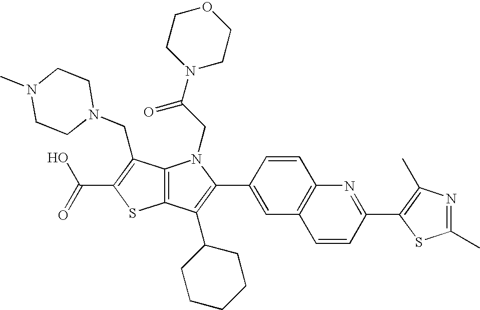 | 6-Cyclohexyl-5-[2-(2,4-dimethyl- thiazol-5-yl)-quinolin-6-yl]-3-(4- methyl-piperazin-1-ylmethyl)-4-(2- morpholin-4-yl-2-oxo-ethyl)-4H- thieno[3,2-b]pyrrole-2-carboxylic acid |
| 182 | 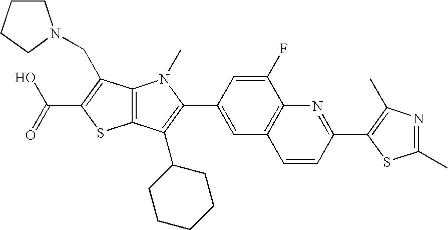 | 6-Cyclohexyl-5-[2-(2,4-dimethyl- thiazol-5-yl)-8-fluoro-quinolin-6- yl]-4-methyl-3-pyrrolidin-1- ylmethyl-4H-thieno[3,2-b]pyrrole- 2-carboxylic acid |
| 183 | 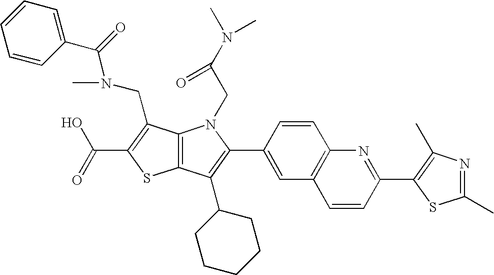 | 3-[(Benzoyl-methyl-amino)- methyl]-6-cyclohexyl-4- dimethylcarbamoylmethyl-5-[2- (2,4-dimethyl-thiazol-5-yl)- quinolin-6-yl]-4H-thieno[3,2- b]pyrrole-2-carboxylic acid |
| 184 | 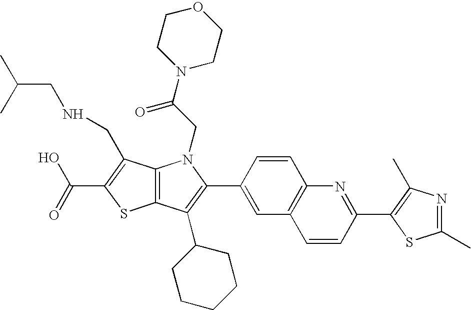 | 6-Cyclohexyl-5-[2-(2,4-dimethyl- thiazol-5-yl)-quinolin-6-yl]-3- (isobutylamino-methyl)-4-(2- morpholin-4-yl-2-oxo-ethyl)-4H- thieno[3,2-b]pyrrole-2-carboxylic acid |
| 185 | 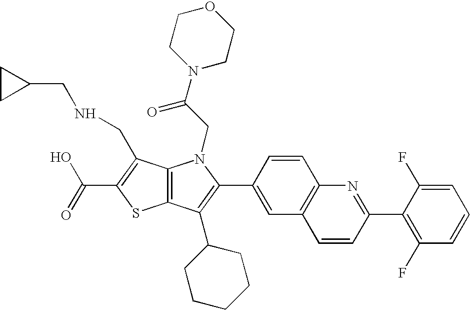 | 6-Cyclohexyl-3- [(cyclopropylmethyl-amino)- methyl]-5-[2-(2,6-difluoro-phenyl)- quinolin-6-yl]-4-(2-morpholin-4-yl- 2-oxo-ethyl)-4H-thieno[3,2- b]pyrrole-2-carboxylic acid |
| 186 | 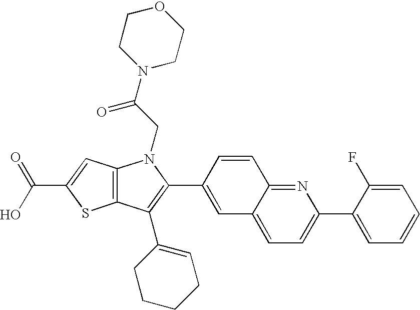 | 6-Cyclohex-1-enyl-5-[2-(2-fluoro- phenyl)-quinolin-6-yl]-4-(2- morpholin-4-yl-2-oxo-ethyl)-4H- thieno[3,2-b]pyrrole-2-carboxylic acid |
| 187 | 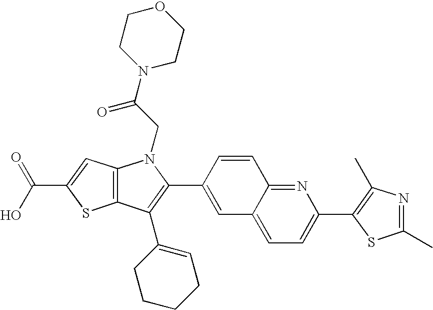 | 6-Cyclohexyl-1-enyl-5-[2-(2,4- dimethyl-thiazol-5-yl)-quinolin-6- yl]-4-(2-morpholin-4-yl-2-oxo- ethyl)-4H-thieno[3,2-b]pyrrole-2- carboxylic acid |
| 188 | 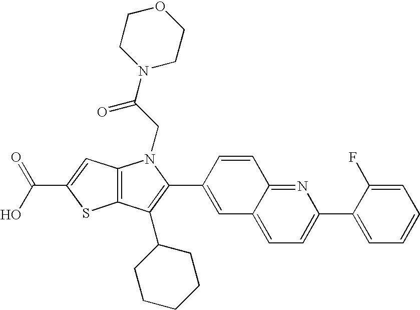 | 6-Cyclohexyl-5-[2-(2-fluoro- phenyl)-quinolin-6-yl]-4-(2- morpholin-4-yl-2-oxo-ethyl)-4H- thieno[3,2-b]pyrrole-2-carboxylic acid |
| 189 | 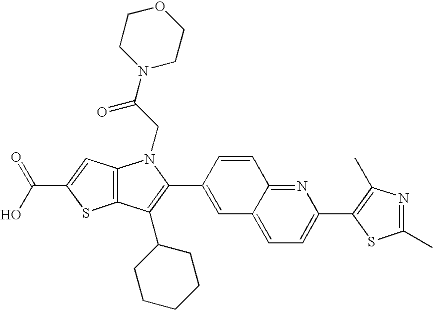 | 6-Cyclohexyl-5-[2-(2,4-dimethyl- thiazol-5-yl)-quinolin-6-yl]-4-(2- morpholin-4-yl-2-oxo-ethyl)-4H- thieno[3,2-b]pyrrole-2-carboxylic acid |
| 190 | 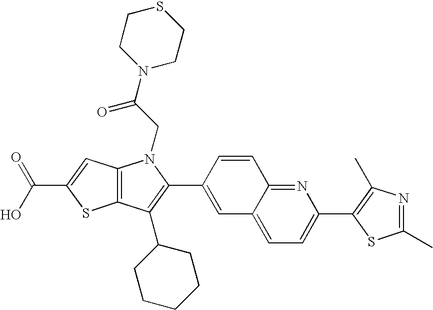 | 6-Cyclohexyl-5-[2-(2,4-dimethyl- thiazol-5-yl)-quinolin-6-yl]-4-(2- oxo-2-thiomorpholin-4-yl-ethyl)- 4H-thieno[3,2-b]pyrrole-2- carboxylic acid |
| 191 | 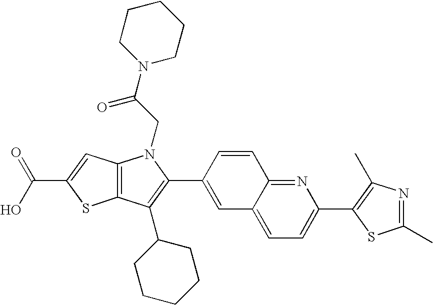 | 6-Cyclohexyl-5-[2-(2,4-dimethyl- thiazol-5-yl)-quinolin-6-yl]-4-(2- oxo-2-piperidin-1-yl-ethyl)-4H- thieno[3,2-b]pyrrole-2-carboxylic acid |

The present invention further provides metabolites of any of compounds of Formula (I), (Ia)-(Is), or of the compounds in Table 1. In some aspects, the metabolite is an oxide.

This invention is also directed to pharmaceutical compositions comprising a pharmaceutically acceptable diluent and a therapeutically effective amount of one of the compounds described herein or mixtures of one or more of such compounds.

This invention is further directed to methods for treating a viral infection mediated at least in part by a virus in the Flaviviridae family of viruses, such as HCV, in mammals which methods comprise administering to a mammal, that has been diagnosed with said viral infection or is at risk of developing said viral infection, a pharmaceutical composition comprising a pharmaceutically acceptable diluent and a therapeutically effective amount of one of the compounds described herein or mixtures of one or more of such compounds. In another aspect, present invention provides for use of the compounds of the invention for the preparation of a medicament for treating or preventing said infections. In other aspects the mammal is a human.

In yet another embodiment of the invention, methods of treating or preventing viral infections in mammals are provided where in the compounds of this invention are administered in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. Preferably, the additional agent active against HCV is interferon-alpha or pegylated interferon-alpha alone or in combination with ribavirin or viramidine.

General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

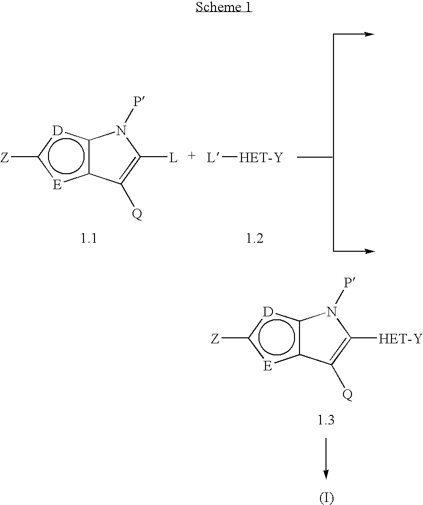

In one embodiment, the compounds of Formula (I) are prepared via a transition metal catalyzed cross-coupling reaction as shown above in Scheme 1 where L and L′ are suitable cross-coupling substituents, P′ is hydrogen, a nitrogen protecting group, or R, and Z, D, E, R, Q, HET, and Y are as previously defined. Typically, one of L or L′ is a Sn, B, Zr, or Zn based metal (e.g. —BOH₂, Sn(CH₃)₃, etc.) and the other of L or L′ is a leaving group such as halogen or sulfonate. Suitable halogens and sulfonates include Cl, Br, I, —OSO₂CF₃, and —OSO₂CH₃. Suitable transition metal catalysts include Pd and Ni based catalysts (e.g. Pd(PPh₃)₂Cl₂, Pd[P(Ph₃)]₄, etc.). In one embodiment, one of 1.1 or 1.2 has L is —B(OH)₂ and is prepared by treating a compound of 1.1 or 1.2 where L or L′ is halogen with an excess of bis(neopentylglycolato)diboron in the presence of a catalytic amount of triphenylphosphine palladium(II) dichloride. The resulting boronic acid is the coupled with the other of 1.1 or 1.2 where L is halogen or a sulfonate under Suzuki coupling conditions to form a compound of Formula (I) or an intermediate 1.3. Suitable coupling conditions include reaction of 1.1 and 1.2 in refluxing methanol containing Pd[P(Ph)₃]₄ and NaHCO₃ for 10 to 20 hours. When P′ is H or a protecting group, removal of the protecting group followed by functionalization of the resulting NH group yields compound (I). An specific example of this transformation is shown in Scheme 5.

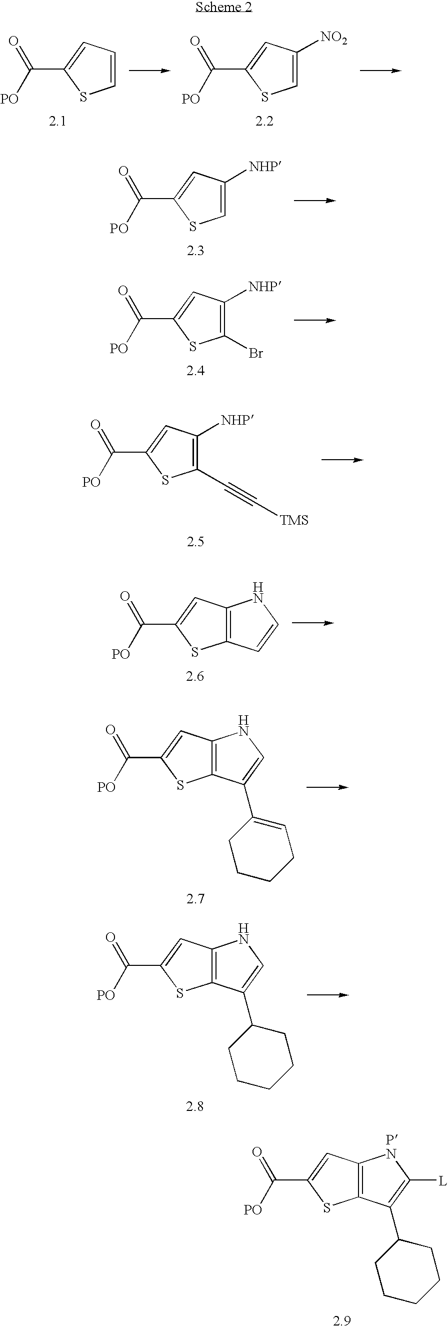

In one embodiment, compound 1.1 can be synthesized as shown in Scheme 2 where for illustrative purposes D is CH, E is S, Z is COOP, Q is cyclohexyl, P is a hydroxy protecting group such as alkyl, P′ is a nitrogen protecting group, and L is halogen. Thiophene 2.1 is treated with a mixture of nitric and sulfuric acid to form nitro compound 2.2. Reduction of the nitro group followed by protection of the resulting amine with a protecting group P′ such as t-butyloxycarbonyl affords compound 2.3. Thiophene 2.3 can be treated with a halogenating agent such as N-bromosuccinimide (NBS) to form bromide 2.4. Exposure of 2.4 with trimethylsilylacetylene, CuI, and PdCl₂(PPh₃)₂ gives acetylene 2.5 that is then treated with n-Bu₄NF and exposed to microwave radiation to form 2.6. Compound 2.6 is next reacted with cyclohexanone and sodium ethoxide in ethanol under refluxing conditions to form cyclohexene 2.7 that is then reduced to cyclohexane 2.8 with H₂ and Pd(OH)₂/C or with a reducing agent such as triethylsilane. Compound 2.8 can then be functionalized to introduce group R, or the ring nitrogen can be protected followed treatment with a halogenating agent such as NBS to form the coupling partner 2.9.

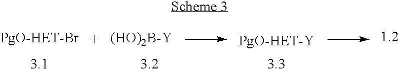

The L′-HET-Y group 1.2 described in Scheme 1 can be prepared by conventional procedures well known in the art. Scheme 3 illustrates one generic method for preparing suitable HET-Y groups for use in such convergent synthesis. Scheme 3 employs a bromo and hydroxyl substituted aryl or heteroaryl compound 3.1, which is optionally further substituted with one or more X groups (not shown). If necessary, the hydroxyl group can be protected by conventional protecting groups, Pg, which are well known in the art. Compound 3.3 is formed by reacting 3.1 under conventional Suzuki conditions with the boronic acid 3.2, which can be prepared in the manner described in Scheme 1 above from the corresponding Y—Br compound. When Pg is not hydrogen, the protecting group can be removed by conventional procedures. The resulting hydroxyl group of compound 3.3 can next be converted under conventional conditions to compound 1.2 for use in the coupling step of Scheme 1.

Scheme 4 below illustrates the preparation of quinolinyl HET-Y group having a bromo group suitable for Suzuki coupling with compound 1.1. It is understood that this quinolinyl group is depicted for illustrative purpose only.

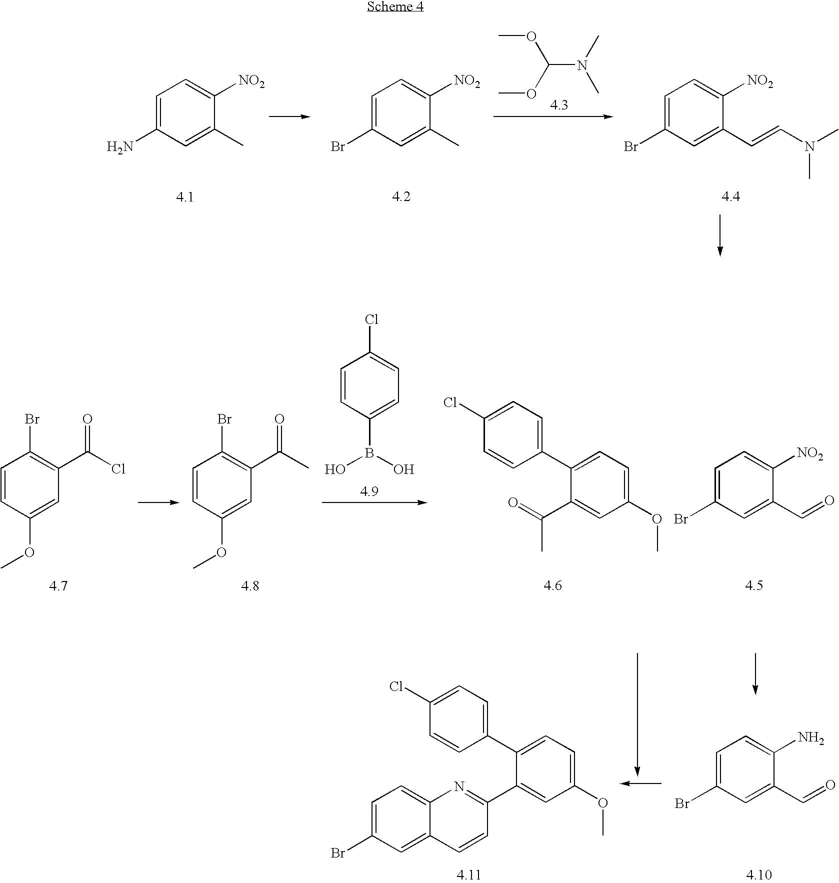

In Scheme 4, commercially available amino 2-methyl-4-nitrobenzene, compound 4.1, is converted to the corresponding bromo-2-methyl-nitrobenzene, compound 4.2, under conventional conditions using an equimolar amounts of sodium nitrite, an excess of HBr and a catalytic amount of cupric bromide. The reaction is preferably conducted by combining compound 4.1 with an excess of aqueous hydrogen bromide (e.g., 48% HBr) in an inert solvent at a temperature of from about −10 to 10° C. An equimolar amount of sodium nitrite dissolved in water is slowly added to the reaction mixture while maintaining the reaction temperature. A catalytic amount of solid cuprous bromide is then added to the reaction mixture and the reaction mixture is allowed to warm to slightly less than room temperature. The reaction is monitored until nitrogen evolution ceases indicating reaction completion. Afterwards, the resulting product, bromo-2-methyl-nitrobenzene, compound 4.2, can be isolated by conventional techniques such as evaporation, extraction, precipitation, filtration, chromatography, and the like; or, alternatively, used in the next step without purification and/or isolation.

Suitable examples of compound 4.1 include commercially available variants such as 2-nitro-3-methylaniline, 4-methyl-3-nitroaniline (both commercially available from Aldrich Chemical Company, Milwaukee, Wis., USA) as well as 3-methyl-4-nitroaniline (commercially available from Lancaster Synthesis Inc.).

Compound 4.2 is next converted to (E)-2-(bromo-2-nitrophenyl)vinyl dimethylamine, compound 4.4, by reaction with an excess of N,N-dimethylformamide dimethylacetal, compound 4.3. The reaction is typically conducted in a suitable solvent such as DMF under an inert atmosphere. Preferably, the reaction is conducted at an elevated temperature of from about 100° C. to about 160° C. The reaction is continued until it is substantially complete which typically occurs within about 1 to 6 hours. After reaction completion, the resulting product can be isolated by conventional techniques such as evaporation, extraction, precipitation, filtration, chromatography, and the like; or, alternatively, used in the next step without purification and/or isolation.

Oxidation of (E)-2-(bromo-2-nitrophenyl)vinyl dimethylamine, compound 4.4, proceeds via contact with a large excess of sodium periodate to provide for bromo-2-nitrobenzaldehyde. This reaction is typically conducted in an inert diluent such as an aqueous mixture of tetrahydrofuran, dioxane, and the like. Preferably, the reaction is conducted at an ambient conditions and is continued until it is substantially complete which typically occurs within about 0.5 to 6 hours. After reaction completion, the resulting product, bromo 2-nitrobenzaldehyde, compound 4.5, can be isolated by conventional techniques such as evaporation, extraction, precipitation, filtration, chromatography, and the like; or, alternatively, used in the next step without purification and/or isolation.

Conventional reduction of compound 4.5 provides for the corresponding bromo 2-aminobenzaldehyde, compound 4.10.

Separately, bromo-5-methoxybenzoyl chloride, compound 4.7 (available from Maybridge), is converted to the corresponding bromo-3-acetyl-methoxybenzene, compound 4.8, by reaction with dimethyl zinc. The reaction is typically conducted in a suitable inert diluent such as benzene, toluene, xylene and the like. Preferably, the dimethyl zinc is present in the solvent prior to addition of compound 4.7 as dimethyl zinc is pyroforic. Preferably, the reaction is initially conducted at a temperature of from about −10 to about 10° C. and then allowed to slowly proceed to room temperature. The reaction is continued until it is substantially complete which typically occurs within about 0.2 to 2 hours. After reaction completion, the resulting product, bromo-3-acetyl-methoxy-benzene (compound 4.8) can be isolated by conventional techniques such as evaporation, extraction, precipitation, filtration, chromatography, and the like; or, alternatively, used in the next step without purification and/or isolation.

Alternatively, bromo-5-methoxybenzoyl chloride, compound 4.7, can be prepared from the corresponding commercially available bromo-5-methoxybenzoic acid such as 2-bromo-5-methoxybenzoic acid (available from Aldrich Chemical Company, Milwaukee, Wis., USA) by conversion into an acid halide. The acid halide can be prepared by contacting the carboxylic acid with an inorganic acid halide, such as thionyl chloride, phosphorous trichloride, phosphorous tribromide or phosphorous pentachloride, or preferably, with oxalyl chloride under conventional conditions. Generally, this reaction is conducted using about 1 to 5 molar equivalents of the inorganic acid halide or oxalyl chloride, either neat or in an inert solvent, such as dichloromethane or carbon tetrachloride, at temperature in the range of about 0° C. to about 80° C. for about 1 to about 48 hours. A catalyst, such as DMF, may also be used in this reaction.

Commercially available chlorophenyl boronic acid, compound 4.9, is coupled with compound 4.8 via conventional Suzuki conditions to provide for chlorophenyl substituted 3-acetyl methoxybenzene, compound 4.6. 2-, 3- And 4-chlorophenyl boronic acids are commercially available from Aldrich Chemical Company, supra.

Compound 4.6 is then coupled with compound 4.10 under condensation conditions to provide for 2-biaryl-6-bromoquinoline, compound 4.11. This reaction is preferably conducted by combining approximately stoichiometric amounts of both compounds 4.6 and 4.10 in a suitable inert diluent such as ethanol, isopropanol and the like in the presence of a suitable base such as potassium hydroxide under an inert atmosphere. Preferably, the reaction is conducted at a temperature of from about 70° C. to about 100° C. and proceeds until it is substantially complete which typically occurs within about 2 to 16 hours. After reaction completion, the resulting product, compound 4.11, can be isolated by conventional techniques such as evaporation, extraction, precipitation, filtration, chromatography, and the like; or, alternatively, used in the next step without purification and/or isolation.

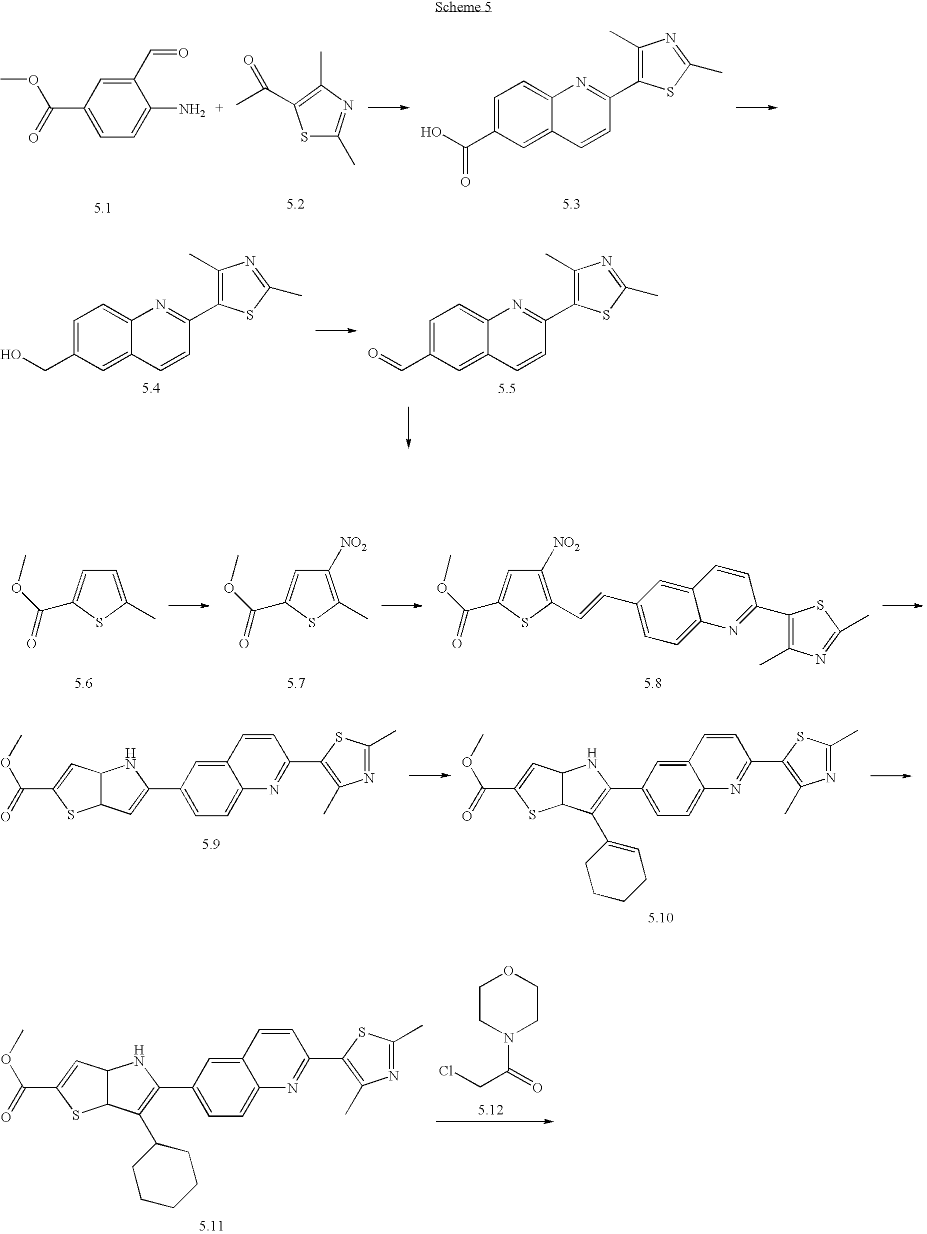

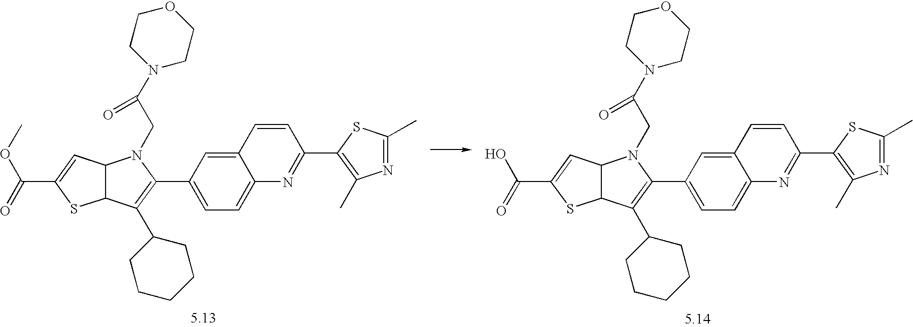

In addition to the synthesis described in Scheme 1, compounds of Formula (I) can also be prepared by other methods. Scheme 5 shows one such method where for illustrative purposes Z is COOH, D is CH, E is S, Q is cyclohexyl, and the R^{a}, and HET-Y groups are as depicted in compound 5.14. Compound 5.1 is condensed with the commercially available (Aldrich) 5.2 using the Friedlander conditions to form quinoline 5.3. An example of such conditions is given in Example 2 below. Compound 5.3 can be converted to the corresponding alcohol 5.4 using known methods such as with lithium aluminum hydride followed by re-oxidation to aldehyde 5.5 using Dess-Martin reagent. Commercially available thiophene 5.6 is converted to 5.7 by treatment with nitric acid/sulfuric acid. Compounds 5.7 and 5.5 are then refluxed together in MeOH in the presence of catalytic amount of pyrrolidine to form the nitro-olefin 5.8. Compound 5.8 is next refluxed with triethyl phosphite to afford thieno-pyrrole derivative 5.9. The cyclohexyl ring is introduced as in Scheme 2 by heating 5.9 with cyclohexanone in the presence of acetic acid, acetic anhydride, and phosphoric acid to give 5.10. Reduction of compound 5.10 with triethylsilane gives 5.11. The acetamido moiety is introduced by reacting 5.11 with the commercially available 5.12 in DMF using standard alkylating conditions to form 5.13 which is saponified with aqueous LiOH to give the desired product 5.14.

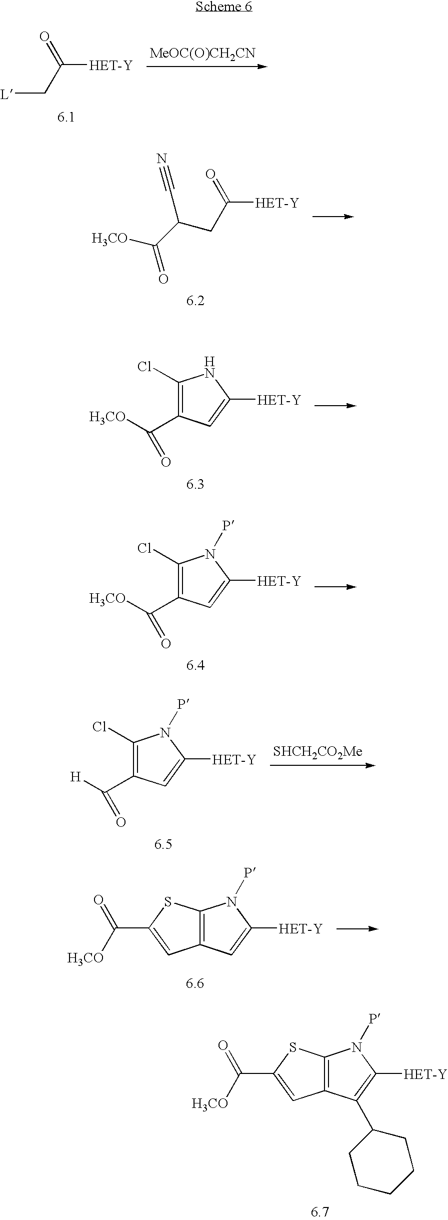

In another embodiment, compounds of Formula (I) are synthesized as shown in Scheme 6 where for illustrative purposes D is S, E is CH, Z is COOP, Q is cyclohexyl, P is a hydroxy protecting group such as alkyl, P′ is a nitrogen protecting group, L′ is a leaving group such as halogen, and HET and Y are previously defined. Compound 6.1 is reacted with methyl cyanoacetate in the presence of a base such as diisopropylethyl amine to form alkylated product 6.2. Exposure of 6.2 to HCl gas affords the pyrrole 6.3 which can then be converted to a protected pyrrole such as 6.4 where P′ is benzyl by reaction with benzyl bromide and NaH. Ester 6.4 is next converted to aldehyde 6.5 such as by a two step procedure of reduction of 6.4 with diisobutylaluminum hydride to the corresponding alcohol followed by oxidation to aldehyde 6.5 with an oxidizing agent such as (n-Pr)₄N RuO₄/N-methylmorpholine N-oxide. Reaction of aldehyde 6.5 with methyl thioglycolate and potassium tert-butoxide in THF gives compound 6.6, which can be functionalized to give 6.7 in a similar manner as described in Scheme 5 to introduce the cyclohexyl moiety. Likewise, the P′ protecting group can be removed from 6.7 and the R group can be introduced as described in Scheme 5 to give a compound of Formula (I).

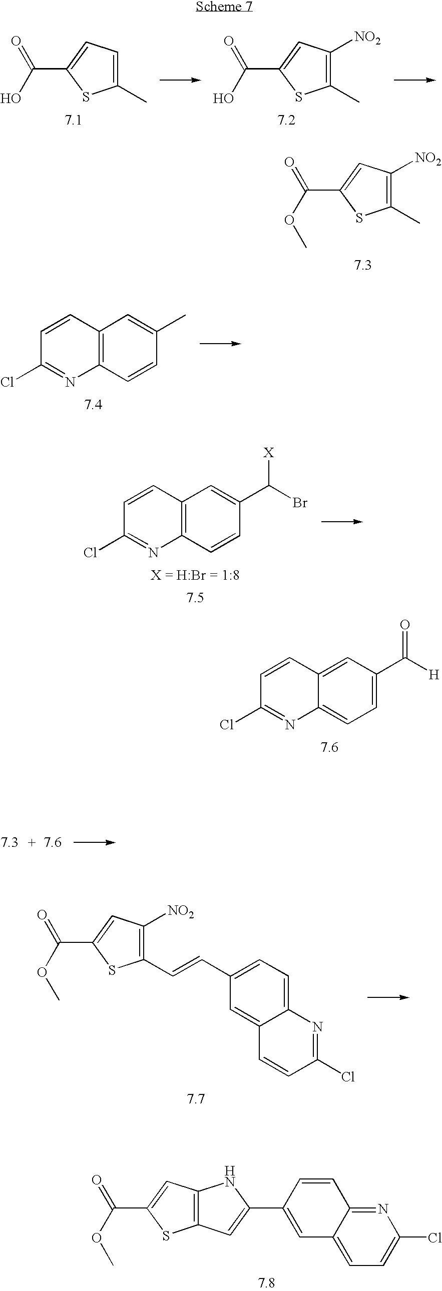

Scheme 7 illustrates the synthesis of intermediate 7.8 formed from coupling nitro compound 7.3 with aldehyde 7.6. Nitration of thiophene 7.1 under suitable nitration conditions such as by addition to a solution of acetic anhydride and nitric acid forms acid 7.2 that is then esterified to give intermediate ester 7.3. Coupling partner 7.6 is prepared starting from 2-chloro-6-methylquinoline 7.4 that is halogenated upon treatment with a suitable halogenating reagent such as NBS (N-bromosuccinimide) to give a mixture of mono and dibromides 7.5. The mixture is then refluxed in an aqueous solvent such as 50% aq. ethanol in the presence of an amine such as hexamethylenetetramine to give, following an acidic work-up, aldehyde 7.6. Nitro compound 7.3 and aldehyde 7.6 are reluxed together in an alcoholic solvent such as methanol to which is added a catalytic amount of an amine such as pyrrolidine to give olefin 7.7, that is then treated with triethyl phosphite to form the cyclized thienopyrrole 7.8. Details of the preparation of 7.8 is given in Example 4.

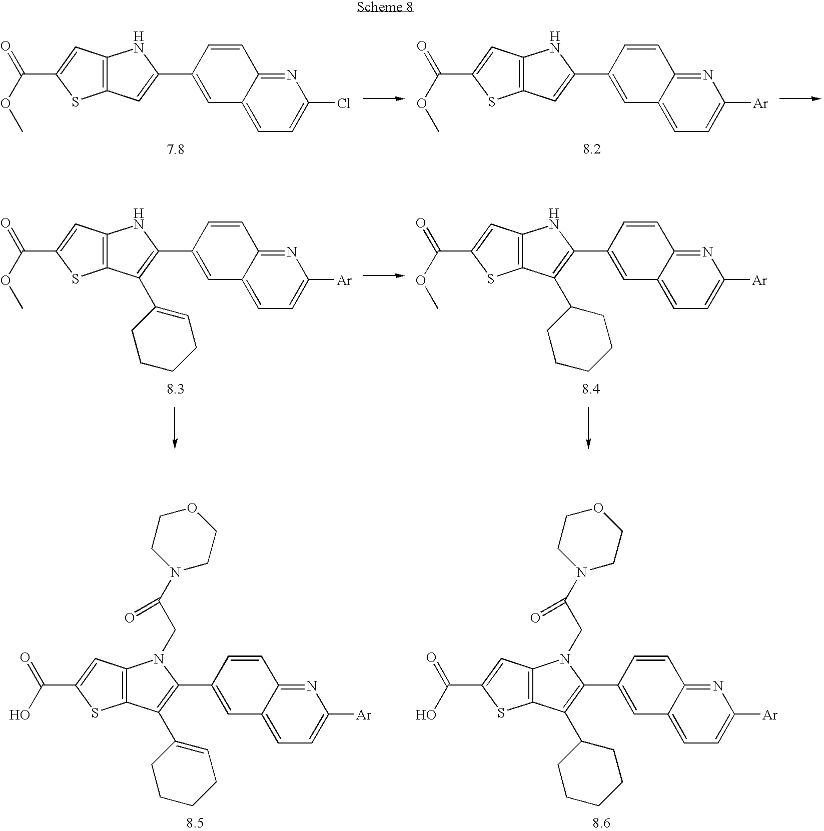

Scheme 8 illustrates the use of intermediate 7.8 to prepare compounds 8.2-8.6 following the methods described in Scheme 5.

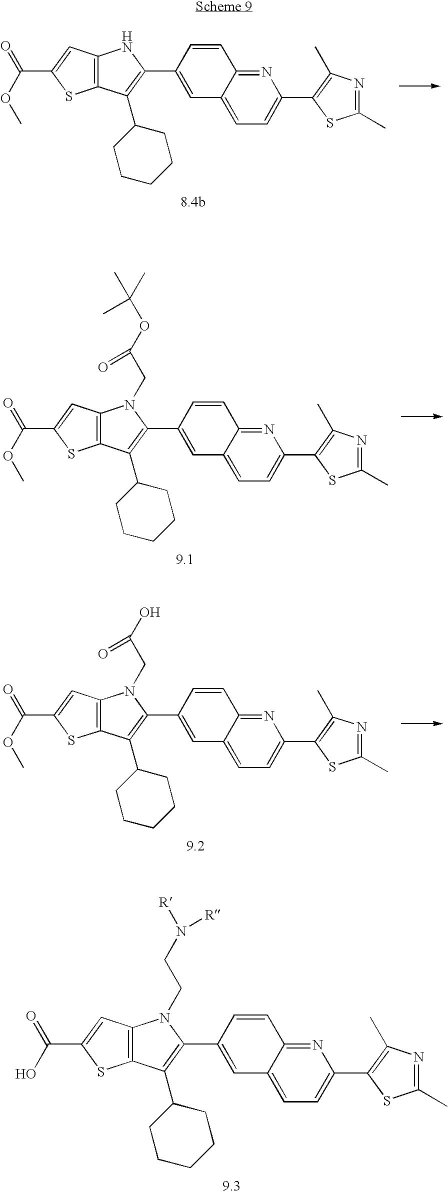

Scheme 8 illustrates the preparation of compounds such as 9.3 following the methods described in the Schemes above. An example of the synthesis of compound 9.3 wherein R′ and R″ together form a cyclic group is given in Examples 9 and 10.

Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.01-25 mg/kg/day, more preferably from about 0.1 to 10 mg/kg/day. Thus, for administration to a 70 kg person, the dosage range would most preferably be about 7-70 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

In still other embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In another embodiments, the compound having anti-HCV activity is said agent active against HCV is interferon-alpha or pegylated interferon-alpha alone or in combination with Ribavirin or viramidine.

### EXAMPLES

In the examples below and the synthetic schemes above, the following abbreviations have the following meanings. If an abbreviation is not defined, it has its generally accepted meaning.

| | |
|---|---|
| μL = | microliters |
| μM = | micromolar |
| μg = | micrograms |
| NMR = | nuclear magnetic resonance |
| br = | broad |
| d = | doublet |
| δ = | chemical shift |
| dd = | doublet of doublets |
| DMEM = | Dulbeco's Modified Eagle's Medium |
| DMF = | N,N-dimethylformamide |
| DMSO = | dimethylsulfoxide |
| DTT = | dithiothreotol |
| EDTA = | ethylenediaminetetraacetic acid |
| ESI = | electrospray ionization |
| g = | gram |
| h or hr = | hours |
| HCV = | hepatitus C virus |
| HPLC = | high performance liquid chromatography |
| Hz = | hertz |
| IPTG = | isopropyl-β-D-thiogalactopyranoside |
| IU = | International Units |
| IC₅₀ = | inhibitory concentration at 50% inhibition |
| J = | coupling constant (given in Hz unless otherwise |
| | indicated) |
| m = | multiplet |
| M = | molar |
| M+ H⁺ = | parent mass spectrum peak plus H⁺ |
| mg = | milligram |
| mL = | milliliter |
| mM = | millimolar |
| mmol = | millimole |
| MS = | mass spectrum |
| nm = | nanometer |
| nM = | nanomolar |
| ng = | nanogram |
| NTA = | nitrilotriacetic acid |
| NTP = | nucleoside triphosphate |
| PCR = | Polymerase chain reaction |
| ppm = | parts per million |
| psi = | pounds per square inch |
| Rp-HPLC = | reversed phase high performance liquid |
| | chromatography |
| s = | singlet |
| t = | triplet |
| TC₅₀ = | Toxic concentration at 50% cell toxicity |
| tetrakis or tetrakis = | tetrakis(triphenylphosphine)palladium(0) |
| palladium | |
| TFA = | trifluoroacetic acid |
| THF = | tetrahydrofuran |
| Tris = | Tris(hydroxymenthyl)aminomethane |
| UTP = | uridine triphosphate |

Set forth in the examples below are compounds and intermediates useful for making compounds of the present invention. An overview of the synthetic protocols employed to prepare these compounds is set forth above.

### Example 1

### 6-bromo-2-(4′-chloro-4-methoxy-biphenyl-2-yl)-quinoline 4.11

### Step 1. 4-bromo-2-methyl-1-nitro-benzene (4.2)

To an ice cold solution of 10.0 g (65.7 mmol) 3-methyl-4-nitro-phenylamine in 200 mL acetone, was added 21 mL (197.2 mmol) 48% HBr. 4.54 g (65.7 mmol) NaNO₂ was dissolved in 20 mL water and was added dropwise to the amine solution at a rate to keep the temperature under 5° C. The mixture was stirred at this temperature for an additional 10 minutes then 1.5 g (10 mmol) solid CuBr was added portion-wise at a rate to keep the temperature under 15° C. The reaction was complete when no more nitrogen evaluated (about 15 minutes). The reaction mixture was evaporated to dryness; the residue was dissolved in a mixture of 500 mL water and 750 mL ethyl acetate. The organic phase was separated, washed with water (2×), saturated NaCl (2×) and was dried (Na₂SO₄). It was then evaporated to dryness to give the crude product as a yellow solid which was purified by filtering through 400 mL silica gel pad using toluene elution;

Yield: 10.45 g (73%);

¹H-NMR (CDCl₃): δ (ppm) 7.87 (d, 1H, J=8.7 Hz), 7.51-7.46 (m, 2H), 2.61 (s, 3H).

### Step 2. [(E)-2-(5-bromo-2-nitro-phenyl)-vinyl]-dimethyl-amine (4.4)

A mixture of 9.26 g (42.9 mmol) of compound 4.2, 14.3 mL (107.2 mmol) N,N-dimethylformamide dimethylacetal 4.3 and 11 mL DMF was heated under a slow argon flow at 145° C. (bath) for two hours. The reaction mixture was then evaporated to dryness. The dark pink product crystallized upon standing; MS: 271.01 & 273.01 (M+H⁺); ¹H-NMR (DMSO-d₆): δ (ppm) 7.88 (d, 1H), 7.68 (dd, 1H), 7.58 (d, 1H), 7.05 (d, 1H), 5.59 (d, 1H), 2.90 (s, 6H).

### Step 3. 5-bromo-2-nitro-benzaldehyde (4.5)

Compound 4.4 (11.63 g (42.9 mmol)) was dissolved in 500 mL 1:1 mixture of THF and water. To this solution 34.3 g (160 mmol) NaIO₄ was added and the mixture was stirred at room temperature for 1 hr while the dark solution became pale yellow with a heavy precipitate. The solid material was filtered off, washed twice with 100 mL ethyl acetate and the organic phases were pooled and evaporated to dryness. The residue was filtered through a 400 mL silicagel pad using toluene for elution to get 7.08 g (71%) of the title compound; ¹H-NMR (DMSO-d₆): δ (ppm) 10.10 (s, 1H), 8.09-7.99 (m, 3H).

### Step 4. 2-amino-5-bromo-benzaldehyde (4.10)

Compound 4.10 was synthesized from 5.45 g (23.7 mmol) of compound 4.5 using the procedure of L. I. Smith and J. W. Opie (Org. Synth. Coll. Vol. 3, 56) in 55% yield (2.6 g); MS: 199.97 & 201.97 (M+H⁺); ¹H-NMR (CDCl₃): δ (ppm) 9.75 (s, 1H), 7.71 (s, 1H), 7.39 (d, 1H, J=9.3 Hz), 7.22 (s, 2H), 6.72 (d, 1H, J=9.3 Hz).

### Step 5. 1-(2-bromo-5-methoxy-phenyl)-ethanone (4.8)

To an ice cold solution of 8.75 g (35 mmol) 2-bromo-5-methoxy-benzoyl chloride in 40 mL toluene, 9.63 mL (19.25 mmol) of a 2M toluene solution of dimethylzinc was added under argon atmosphere (dimethylzinc is pyrophoric—contact with air should be avoided!). The ice bath was removed and the mixture slowly warmed up to room temperature. Once the reaction starts it proceeds rapidly resulting in a turbid solution. The reaction was complete in 30 minutes. It was then cooled back to 0° C. and was quenched by adding 10 mL ethanol. The mixture was evaporated to dryness, the residue was dissolved in a mixture of 50 mL 1M HCl and 100 mL ethyl acetate. The organic phase was separated and washed with 50 mL water (2×), brine (2×) and was dried (Na₂SO₄). The final solution was evaporated and the oil dried overnight in high vacuum to give 7.96 g (99%) of the title compound as a colorless liquid; ¹H-NMR (CDCl₃): δ (ppm) 7.46 (d, 1H), 6.96 (d, 1H), 6.83 (dd, 1H), 3.80 (s, 3H), 2.63 (s, 3H).

### Step 6. 1-(4′-chloro-4-methoxy-biphenyl-2-yl)-ethanone (4.6)

A mixture of compound 4.8 (6.0 g, 26.19 mmol), 4-chlorobenzeneboronic acid (4.51 g, 28.81 mmol) and Pd(PPh₃)₄ (0.303 g, 0.262 mmol) in toluene (250 mL), MeOH (60 mL) and 2 M NaHCO₃ (25 mL) was stirred under argon at 80° C. for 16 h. After removal of the solvent, the dry residue was dissolved in CHCl₃ (150 mL) and filtered. The solvent was evaporated and the residue was purified by chromatography using CHCl₃-MeOH (70:1) as eluent to give the title compound (6.33 g, 93%); ¹H NMR (CDCl₃): 7.36 (d, 2H, J=8.4 Hz), 7.27-7.21 (m, 4H), 7.02 (d, 1H, J=2.7 Hz), 3.86 (s, 3H), 2.05 (s, 3H). MS (ESI) 261.07 (M+H).

### Step 7. 6-bromo-2-(4′-chloro-4-methoxy-biphenyl-2-yl)-quinoline (4.11)

Compound 4.11 (100 mg (0.5 mmol)) and compound 4.6 (130 mg (0.5 mmol)) were dissolved in 5 mL ethanol, 800 μL 10% KOH (1.5 mmol) was added and the mixture was kept in a 90° C. bath under argon overnight. The solvent was evaporated and the residue triturated with water. The semi solid compound 4.11 was purified on a 400 mL silica gel pad using toluene for elution to give 2.03 g (44%) yellow gummy material; MS: 424.03 & 426.03 (M+H⁺); ¹H-NMR (DMSO-d₆): δ (ppm) 8.20 (d, 1H, J=2.1 Hz), 8.10 (d, 1H, J=9.0 Hz), 7.93-7.83 (m, 2H), 7.40 (d, 1H, J=8.4 Hz), 7.26-7.23 (m, 3H0, 7.16-7.03 (m, 4H), 3.85 (s, 3H).

### Example 2

### Step 1. 6-Bromo-2-(2,4-dimethyl-thiazol-5-yl)-quinoline

To a solution of KOH (10.32 (85%) g, 156.27 mmol) in anhydrous EtOH (700 mL) was added 2-amino-5-bromobenzaldehyde (10.42 g, 52.09 mmol) and 5-acetyl-2,4-dimethylthiazole (8.16 mL, 60.42 mmol). The mixture was stirred under Ar at 78° C. for 16 h and then cooled down in an ice-bath. It was neutralized to pH 7 with 5 N HCl and then evaporated to about 60 mL. Water (500 mL) was added. The precipitate formed were collected by filtration, washed thoroughly with water, and dried to give 6-bromo-2-(2,4-dimethyl-thiazol-5-yl)-quinoline (15.62 g, 94%).

### Step 2. 2-(2,4-Dimethyl-thiazol-5-yl)-quinoline-6-boronic acid

A mixture of 6-bromo-2-(2,4-dimethyl-thiazol-5-yl)-quinoline (15 g, 46.99 mmol), bis(neopentylglucolato)diboron (31.83 g, 141 mmol), bis(triphenylphosphine)-palladium (II) chloride (1.65 g, 2.35 mmol), and potassium acetate (13.81 g, 141 mmol) in anhydrous DMSO (260 mL) was stirred under Ar at 90° C. for 2 h then was cooled down to room temperate. The mixture was poured into water (1.2 L) and the precipitate were collected by filtration, washed with water, and dried. To the dried solid was added EtOAc (600 mL) and the insoluble solid was filtered off. The filtrate was evaporated and the product was adsorbed onto silica gel and purified by a short silica pad eluting with EtOAc-hexane (5:2) to give 2-(2,4-dimethyl-thiazol-5-yl)-quinoline-6-boronic acid (16.4 g, still containing about 30% bis(neopentyl glucolato)diboron indicated by NMR-94% yield).

### Example 3

### 2-(2,4-Dimethyl-thiazol-5-yl)-8-fluoro-quinoline-6-boronic acid

### Step 1. 4-Amino-3-fluoro-boronic acid

A mixture of commercially available 4-bromo-2-fluoroaniline (500 mg, 2.6 mmol), potassium acetate (764 mg, 7.8 mmol), [P(Ph₃)]₂Pd(II)Cl₂ (18 mg, 0.026 mmol) and bis(neopentylglycolato)diboron (1.76 g, 7.8 mmol) in 13 mL DMSO was heated at 60° C. under argon overnight. The reaction mixture was diluted with ethyl acetate, washed with water and brine, dried (sodium sulfate), and concentrated. The crude product was purified using RP-HPLC to give 4-amino-3-fluoro-boronic acid.

### Step 2. 4-Amino-3-fluoro-5-iodo-boronic acid

4-Amino-3-fluoro-boronic acid is treated with N-iodosuccinimide in acetic acid. The reaction mixture is diluted with ethyl acetate, washed with water and brine, dried (sodium sulfate), and concentrated to give 4-amino-3-fluoro-5-iodo-boronic acid.

### Step 3. 4-Amino-3-fluoro-5-fromyl-boronic acid

4-Amino-3-fluoro-5-iodo-boronic acid is dissolved in THF while CO is bubbled through the reaction vessel. Tetrakis(triphenylphosphino)palladium is added and the reaction heated to 50° C. Tributyltin hydride is added. The reaction mixture is diluted with ethyl acetate, washed with water and brine, dried (sodium sulfate), concentrated, and purified to give 4-amino-3-fluoro-5-formyl-boronic acid.

### Step 4. 2-(2,4-Dimethyl-thiazol-5-yl)-8-fluoro-quinoline-6-boronic acid

A mixture of compound 4-Amino-3-fluoro-5-formyl-boronic acid, 5-acetyl-2,4-dimethylthiazole, and 10% KOH/ethanol in ethanol is refluxed overnight. The reaction is concentrated, triturated with water, and purified to give 2-(2,4-dimethyl-thiazol-5-yl)-8-fluoro-quinoline-6-boronic acid.

### Example 4

### 5-(2-Chloro-quinolin-6-yl)-4H-thieno[3,2-b]pyrrole-2-carboxylic acid methyl ester (7.8)

### Step 1. 5-Methyl-4-nitro-thiophene-2-carboxylic acid (7.2)

Acetic anhydride (17 mL, 176 mmol, 5 equiv) was cooled to −78° C. in a dry ice/acetone bath and slowly was added fuming nitric acid (6 mL, 113 mmol, 3.2 equiv), and the mixture was warmed to −20° C. 5-Methyl-thiophene-2-carboxylic acid 7.1 (5 g, 35.2 mmol, 1 equiv) was slowly added in small portions (RAPID EXHOTHERM). The temperature fluctuated between −20° C. and +10° C. then stabilized to −20° C. The reaction mixture was stirred at −20° C. for 10 min. Then the reaction mixture was quenched with ice-water to give a precipitate which was collected by filtration and washed with ice-water. The pink solid was recrystallized from EtOH/H₂O. The collected crystals were washed with ice-water, air dried, and dried in vacuo to yield 5-methyl-4-nitro-thiophene-2-carboxylic acid 7.2 (3.24 g, 50%) as a pink-brown solid. The reaction was repeated in 15 gram-scale (yield 9.82 g, 50%). MS: 188.70 (M+H⁺); ¹H-NMR (DMSO-d₆): δ (ppm) 13.77 (bs, 1H), 8.00 (s, 1H), 2.79 (s, 3H).

### Step 2. 5-Methyl-4-nitro-thiophene-2-carboxylic acid methyl ester (7.3)

Compound 7.2 (10 g, 53.4 mmol, 1 equiv) in MeOH (100 mL) was treated with sulfuric acid (10 mL, 19 mmol, 3.5 equiv) and heated to reflux for 1 day. After the reaction mixture was cooled to ambient temperature, the solvent was evaporated. The residue was dissolved in EtOAc and quenched with saturated NaHCO₃, then the layers were separated. The organic layer was washed with brine, dried (Na₂SO₄), filtered, concentrated, and dried in vacuo to yield 5-methyl-4-nitro-thiophene-2-carboxylic acid methyl ester 7.3 (9.95 g, 93%) as a pale-brown solid. MS: 202.00 (M+H⁺); ¹H-NMR (DMSO-d₆): δ (ppm) 8.06 (s, 1H), 3.84 (s, 3H), 2.78 (s, 3H).

### Step 3. 2-Chloro-6-bromomethyl-quinoline and 2-chloro-6-dibromomethyl-quinoline (7.5)

To a solution of 2-chloro-6-methylquinoline 7.4 (2 g, 11.3 mmol, 1 equiv) in benzene (13 mL) under argon was added NBS (4 g, 23 mmol, 2 equiv) followed by benzoyl peroxide (0.365 g, 1.13 mmol, 0.10 equiv). The mixture was heated to reflux for 4 h. After cooling to RT, the solvent was evaporated, and the residue was dissolved in DCM, and washed with saturated NaHCO₃. The organic layer was dried (Na₂SO₄), filtered, and concentrated. The crude product was purified by ISCO (DCM:Hex=4:1) to yield 2-chloro-6-bromomethyl-quinoline and 2-chloro-6-dibromomethyl-quinoline 7.5 (3 g, 80%) as a white solid which consisted of a 1:8 ratio of monobromo-quinoline:dibromo-quinoline as judged by HPLC. TLC gradient DCM:Hex=4:1. 2-Chloro-6-bromomethyl-quinoline: MS: 255.65 & 257.65 (M+H⁺); 2-chloro-6-dibromomethyl-quinoline: MS: 333.80 & 335.80 & 337.80 (M+H⁺).

### Step 4. 2-Chloro-quinoline-6-carbaldehyde (7.6)

The 18 mixture of brominated quinolines 7.5 (3 g, 9.24 mmol, 1 equiv) and hexamethylenetetramine (3.89 g, 28 mmol, 3 equiv) were heated to reflux in 50% aqueous ethanol (16 mL) for 1 h. After cooling to RT, water (10 mL) was added followed by the slow addition of 12N HCl (1.50 mL) over 5 min. The reaction mixture was heated to reflux for 0.5 h then cooled to RT. The reaction mixture was added to brine and extracted with DCM 4×. The collected organics were washed with brine 2×, dried (Na₂SO₄), filtered, and concentrated. The solid was dried in vacuo to yield 2-chloro-quinoline-6-carbaldehyde 7.6 (1.63 g, 92%) as a white solid which was used without further purification. MS: 192.00 (M+H⁺); ¹H-NMR (DMSO-d₆): δ (ppm) 10.17 (s, 1H), 8.69 (m, 1H), 8.68 (d, 1H, J=8.4 Hz), 8.21 (dd, 1H, J=9.0 Hz and 1.8 Hz), 8.09 (dd, 1H, J=8.4 Hz and 0.60 Hz), 7.75 (d, 1H, J=8.4 Hz).

### Step 5. 5-[(E)-2-(2-Chloro-quinolin-6-yl)-vinyl]-4-nitro-thiophene-2-carboxylic acid methyl ester (7.7)

A solution of compound 7.3 (1.71 g, 8.51 mmol, 1 equiv) in MeOH (35 mL) was treated with compound 7.6 (1.63 g, 8.51 mmol, 1 equiv). The reaction mixture was heated to reflux until a solution was obtained. Then a catalytic amount of pyrrolidine (70 μL, 0.0605 g, 0.851 mmol, 0.10 equiv) was added. The reaction mixture was heated to reflux over-night. After cooling to RT, evaporation of the solvent gave a residue that was purified by ISCO (gradient Hex:EtOAc=100:0 to 0:100) to afford 5-[(E)-2-(2-chloro-quinolin-6-yl)-vinyl]-4-nitro-thiophene-2-carboxylic acid methyl ester 7.7 (2.62 g, 82%) as an orange-red solid. TLC gradient Hex:EtOAc=1:1. MS: 375.70 (M+H⁺); ¹H-NMR (DMSO-d₆): δ (ppm) 8.50 (d, 1H, J=8.7 Hz), 8.34 (bs, 1H), 8.18 (m, 1H), 8.16 (bs, 1H), 8.00 (d, 1H, J=9.0 Hz), 7.80 (d, 1H, J=16.5 Hz), 7.65 (d, 1H, J=8.7 Hz), 7.30 (d, 1H, J=17.4 Hz), 3.89 (s, 3H).

### Step 6. 5-(2-Chloro-quinolin-6-yl)-4H-thieno[3,2-b]pyrrole-2-carboxylic acid methyl ester (7.8)

A solution of compound 7.7 (2.62 g, 7.00 mmol, 1 equiv) in triethyl phosphite (7 mL) was heated to reflux (160° C.) for 2 h. After cooling to RT, the solvents [P(OEt)₃ bp 153-157° C.; OP(OEt)₃ bp 215° C.] were evaporated under high vacuum with the water bath maintained at 70° C. The residue was taken up in EtOAc and precipitated with n-hexane. The solid was collected by filtration and washed with 5% EtOAc/n-hexane. After air-drying for a few minutes, the solid was dried in vacuo to afford target compound 7.8 (960 mg, 40%) as a brown-yellow solid. TLC gradient Hex:EtOAc=1:1. MS: 343.00 (M+H⁺); ¹H-NMR (DMSO-d₆): δ (ppm) 12.3 (bs, 1H), 8.39 (m, 2H), 8.24 (d, 1H, J=8.7 Hz), 8.00 (d, 1H, J=8.7 Hz), 7.71 (s, 1H), 7.61 (d, 1H, J=9.0 Hz), 7.16 (s, 1H), 3.82 (s, 3H).

### Example 5

### 6-Cyclohex-1-enyl-5-[2-(2-fluoro-phenyl)-quinolin-6-yl]-4-(2-morpholin-4-yl-2-oxo-ethyl)-4H-thieno[3,2-b]pyrrole-2-carboxylic acid (compound 186)

### Step 1. 5-[2-(2-Fluoro-phenyl)-quinolin-6-yl]-4H-thieno[3,2-b]pyrrole-2-carboxylic acid methyl ester (8.2a)

A microwave reaction vessel was charged with 387 mg (1.13 mmol) compound 7.8 (Example 4), 237 mg (1.69 mmol, 1.5 eq) 2-fluorophenyl boronic acid and 65 mg (0.057 mmol, 0.05 eq) Pd(PPh₃)₄. To this was added 12 mL dioxane and 4 mL 1M aqueous K₃PO₄. The reaction vessel was sealed, and subsequently degassed and purged with Ar (2×). The reaction mixture was then heated by microwave to 120° C. for 10 min. HPLC analysis confirmed complete consumption of compound 7.8. The reaction mixture was allowed to cool to room temperature, during which time a precipitate formed. The precipitate was collected by filtration, washed with cold H₂O and dried under vacuum to afford 399 mg (88%) of 5-[2-(2-fluoro-phenyl)-quinolin-6-yl]-4H-thieno[3,2-b]pyrrole-2-carboxylic acid methyl ester 8.2a as a yellow powder. MS: 403.1 (M+H⁺).

### Step 2. 6-Cyclohex-1-enyl-5-[2-(2-fluoro-phenyl)-quinolin-6-yl]-4H-thieno[3,2-b]pyrrole-2-carboxylic acid methyl ester (8.3a)

A microwave reaction vessel was charged with 245 mg (0.61 mmol) compound 8.2a, 947 μL (9.15 mmol, 15 eq) cyclohexanone, 500 μL acetic anhydride, 500 μL 85% H₃PO₄ and 4 mL acetic acid. The reaction vessel was sealed and heated by microwave to 180° C. for 75 min. HPLC analysis confirmed complete consumption of compound 8.2a. The reaction mixture was poured into 50 mL NH₄OH (conc., aq.) at 0° C. The aqueous mixture was further diluted with H₂O and extracted with ethyl acetate (3×). The combined extracts were then washed with HCl (1M, aq.), NaHCO₃ (sat., aq.) and brine. The organic phase was then dried over Na₂SO₄, filtered and concentrated to afford 6-cyclohex-1-enyl-5-[2-(2-fluoro-phenyl)-quinolin-6-yl]-4H-thieno[3,2-b]pyrrole-2-carboxylic acid methyl ester 8.3a. The crude residue was dried on vacuum and used without further purification. MS: 483.1 (M+H⁺).

### Step 3. 6-Cyclohex-1-enyl-5-[2-(2-fluoro-phenyl)-quinolin-6-yl]-4-(2-morpholin-4-yl-2-oxo-ethyl)-4H-thieno[3,2-b]pyrrole-2-carboxylic acid (compound 186)

A reaction vessel was charged with 75 mg (0.16 mmol) compound 8.3a and dissolved with 8 mL DMF. 11 mg (0.31 mmol, 2 eq) NaH (67% in mineral oil) was then added and the reaction mixture was allowed to stir at room temperature. After 15 min 36 μL (0.31 mmol, 2 eq) of 2-chloro-1-morpholin-4-yl-ethanone was added in 1 portion and the reaction mixture was allowed to continue stirring at room temperature. After 3 h, HPLC and LC-MS analysis confirmed complete consumption of compound 8.3a. The reaction mixture was quenched by adding 0.1 mL H₂O, poured into a 50 mL flask and concentrated. Cold H₂O was then added to the crude residue to precipitate the methyl ester as a dark powder. The solids were collected by centrifuge and washed once more with H₂O. The methyl ester was then transferred to a reaction vial and dissolved with 3 mL THF, 1 mL MeOH and 1 mL LiOH (1M, aq.). The reaction mixture was then heated to 50° C. and carefully monitored by HPLC and LC-MS analysis. Upon complete conversion, the reaction mixture was neutralized with 0.5 mL HCl (2M, aq.) and concentrated. The crude residue was then dissolved with DMF and acidified with TFA. The mixture was then filtered and purified by reverse-phase HPLC to afford 35 mg (37%) of 6-cyclohex-1-enyl-5-[2-(2-fluoro-phenyl)-quinolin-6-yl]-4-(2-morpholin-4-yl-2-oxo-ethyl)-4H-thieno[3,2-b]pyrrole-2-carboxylic acid (compound 186) as an orange powder. MS: 596.2 (M+H⁺); ¹H NMR (DMSO-d6): δ (ppm) 8.55 (d, J=8.1, 1H), 8.17 (d, J=8.7, 1H), 8.09 (td, J=7.9, 1.7, 1H), 8.03-7.99 (m, 2H), 7.91 (s, 1H), 7.73 (dd, J=8.4, 1.7, 1H), 7.65-7.58 (m, 1H), 7.48-7.41 (m, 2H), 5.81-5.78 (m, 1H), 5.00 (s, 2H), 3.48-3.33 (m, 8H), 2.12 (br s, 2H), 1.99 (br s, 2H), 1.54 (br s, 4H).

### Example 6

### 6-Cyclohex-1-enyl-5-[2-(2,4-dimethyl-thiazol-5-yl)-quinolin-6-yl]-4-(2-morpholin-4-yl-2-oxo-ethyl) -4H-thieno[3,2-b]pyrrole-2-carboxylic acid (compound 187)

### Step 1. 5-[2-(2,4-Dimethyl-thiazol-5-yl)-quinolin-6-yl]-4H-thieno[3,2-b]pyrrole-2-carboxylic acid methyl ester (8.2b)

A microwave reaction vessel was charged with 500 mg (1.46 mmol) compound 7.8 (Example 4), 436 mg (1.82 mmol, 1.25 eq) 2,4-dimethyl-thiazole-5-boronic acid pinacol ester and 84 mg (0.073 mmol, 0.05 eq) Pd(PPh₃)₄. To this was added 12 mL dioxane and 4 mL K₃PO₄ (1M, aq.). The reaction vessel was sealed, and subsequently degassed and purged with Ar (2×). The reaction mixture was then heated by microwave to 120° C. for 10 min. HPLC analysis confirmed complete consumption of compound 7.8. The reaction mixture was allowed to cool to room temperature, during which time a precipitate formed. The precipitate was collected by centrifuge, washed with cold H₂O and dried under vacuum to afford 506 mg (81%) of 5-[2-(2,4-dimethyl-thiazol-5-yl)-quinolin-6-yl]-4H-thieno[3,2-b]pyrrole-2-carboxylic acid methyl ester 8.2b as a yellow powder. MS: 420.1 (M+H⁺).

### Step 2. 6-Cyclohex-1-enyl-5-[2-(2,4-dimethyl-thiazol-5-yl)-quinolin-6-yl]-4H-thieno[3,2-b]pyrrole-2-carboxylic acid methyl ester (8.3b)

A microwave reaction vessel was charged with 200 mg (0.48 mmol) compound 8.2b, 740 μL (7.16 mmol, 15 eq) cyclohexanone, 400 μL acetic anhydride, 400 μL 85% H₃PO₄ and 4 mL acetic acid. The reaction vessel was sealed and heated by microwave to 150° C. for 100 min. HPLC analysis confirmed complete consumption of compound 8.2b. The reaction mixture was poured into 50 mL NH₄OH (conc., aq.) at 0° C. The aqueous mixture was further diluted with H₂O and extracted with ethyl acetate (3×). The combined extracts were then washed with HCl (1M, aq.), NaHCO₃ (sat., aq.) and brine. The organic phase was then dried over Na₂SO₄, filtered and concentrated to afford 6-cyclohex-1-enyl-5-[2-(2,4-dimethyl-thiazol-5-yl)-quinolin-6-yl]-4H-thieno[3,2-b]pyrrole-2-carboxylic acid methyl ester 8.3b. The crude residue was dried on vacuum and used without further purification. MS: 500.1 (M+H⁺).

### Step 3. 6-Cyclohex-1-enyl-5-[2-(2,4-dimethyl-thiazol-5-yl)-quinolin-6-yl]-4-(2-morpholin-4-yl-2-oxo-ethyl)-4H-thieno[3,2-b]pyrrole-2-carboxylic acid (Compound 187)

A reaction vessel was charged with 56 mg (0.11 mmol) compound 8.3b and dissolved with 4 mL DMF. 9 mg (0.22 mmol, 2 eq) NaH (60% in mineral oil) was then added and the reaction mixture was allowed to stir at room temperature. After 15 min 26 μL (0.22 mmol, 2 eq) of 2-chloro-1-morpholin-4-yl-ethanone was added in 1 portion and the reaction mixture was allowed to continue stirring at room temperature. After 6 h, HPLC and LC-MS analysis confirmed complete consumption of compound 8.3b. The reaction mixture was quenched by adding 0.1 mL H₂O, poured into a 50 mL flask and concentrated. Cold H₂O was then added to the crude residue to precipitate the methyl ester as a dark powder. The solids were collected by centrifuge and washed once more with H₂O. The methyl ester was then transferred to a reaction vial and dissolved with 3 mL THF, 1 mL MeOH and 1 mL LiOH (1M, aq.). The reaction mixture was then heated to 50° C. and carefully monitored by HPLC and LC-MS analysis. Upon complete conversion, the reaction mixture was neutralized with 0.5 mL HCl (2M, aq.) and concentrated. The crude residue was then dissolved with DMF and acidified with TFA. The mixture was then filtered and purified by reverse-phase HPLC to afford 15 mg (22%) of 6-cyclohex-1-enyl-5-[2-(2,4-dimethyl-thiazol-5-yl) -quinolin-6-yl]-4-(2-morpholin-4-yl-2-oxo-ethyl)-4H-thieno[3,2-b]pyrrole-2-carboxylic acid (Compound 187) as an orange powder. MS: 613.2 (M+H⁺); ¹H NMR (DMSO-d6): δ (ppm) 8.51 (d, J=8.7, 1H), 8.04 (d, J=8.7, 1H), 7.97-7.90 (m, 3H), 7.68 (dd, J=8.7, 2.0, 1H), 5.78 (br s, 1H), 4.99 (s, 2H), 3.46-3.32 (m, 8H), 2.76 (s, 3H), 2.71 (s, 3H), 2.11 (br s, 2H), 1.97 (br s, 2H), 1.53 (br s, 4H).

### Example 7

### 6-Cyclohexyl-5-[2-(2-fluoro-phenyl)-quinolin-6-yl]-4-(2-morpholin-4-yl-2-oxo-ethyl)-4H-thieno[3,2-b]pyrrole-2-carboxylic acid (compound 188)

### Step 1. 6-Cyclohexyl-5-[2-(2-fluoro-phenyl)-quinolin-6-yl]-4H-thieno[3,2-b]pyrrole-2-carboxylic acid methyl ester (8.4a)

A microwave reaction vessel was charged with 235 mg (0.49 mmol) compound 8.3a (Example 5, Step 2), 116 μL (0.73 mmol, 1.5 eq) triethylsilane and 5 mL TFA. The reaction vessel was sealed and heated by microwave to 70° C. for 5 min. LC-MS analysis confirmed complete consumption of compound 8.3a. The reaction mixture was poured into a 50 mL and concentrated to afford 6-cyclohexyl-5-[2-(2-fluoro-phenyl)-quinolin-6-yl]-4H-thieno[3,2-b]pyrrole-2-carboxylic acid methyl ester 8.4a as a red powder. The crude residue was dried on vacuum and used without further purification. MS: 485.1 (M+H⁺).

### Step 2. 6-Cyclohexyl-5-[2-(2-fluoro-phenyl)-quinolin-6-yl]-4-(2-morpholin-4-yl-2-oxo-ethyl) -4H-thieno[3,2-b]pyrrole-2-carboxylic acid (compound 188)

A reaction vessel was charged with 307 mg (0.63 mmol) compound 8.4a and dissolved with 20 mL DMF. 50 mg (1.26 mmol, 2 eq) NaH (60% in mineral oil) was then added and the reaction mixture was allowed to stir at room temperature. After 15 min 146 μL (1.26 mmol, 2 eq) of 2-chloro-1-morpholin-4-yl-ethanone was added in 1 portion and the reaction mixture was allowed to continue stirring at room temperature. After 75 min, HPLC and LC-MS analysis confirmed complete consumption of compound 8.4a. The reaction mixture was quenched by adding 0.5 mL H₂O, poured into a 50 mL flask and concentrated. Cold H₂O was then added to the crude residue to precipitate the methyl ester as a dark powder. The solids were collected by centrifuge and washed once more with H₂O. The methyl ester was then transferred to a reaction vial and dissolved with 6 mL THF, 2 mL MeOH and 2 mL LiOH (1M, aq.). The reaction mixture was then heated to 50° C. and carefully monitored by HPLC and LC-MS analysis. Upon complete conversion, the reaction mixture was neutralized with 1 mL HCl (2M, aq.) and concentrated. The crude residue was then dissolved with DMF and acidified with TFA. The mixture was then filtered and purified by reverse-phase HPLC to afford 107 mg (28%) of 6-cyclohexyl-5-[2-(2-fluoro-phenyl)-quinolin-6-yl]-4-(2-morpholin-4-yl-2-oxo-ethyl)-4H-thieno[3,2-b]pyrrole-2-carboxylic acid (compound 188) as an orange powder. MS: 598.2 (M+H⁺); ¹H NMR (DMSO-d6): δ (ppm) 8.58 (d, J=8.3, 1H), 8.22 (d, J=8.6, 1H), 8.11-7.99 (m, 3H), 7.86 (s, 1H), 7.73 (dd, J=8.6, 2.0, 1H), 7.65-7.58 (m, 1H), 7.48-7.42 (m, 2H), 5.01 (s, 2H), 3.51-3.37 (m, 8H), 2.59 (m, 1H), 2.53-1.25 (m, 10H).

### Example 8

### 6-Cyclohexyl-5-[2-(2,4-dimethyl-thiazol-5-yl)-quinolin-6-yl]-4-(2-morpholin-4-yl-2-oxo-ethyl) -4H-thieno[3,2-b]pyrrole-2-carboxylic acid (compound 189)

### Step 1. 6-Cyclohexyl-5-[2-(2,4-dimethyl-thiazol-5-yl)-quinolin-6-yl]-4H-thieno[3,2-b]pyrrole-2-carboxylic acid methyl ester (8.4b)

A 100 mL round bottom flask was charged with 360 mg (0.72 mmol) compound 8.3b (Example 6, Step 2), 172 μL (1.08 mmol, 1.5 eq) triethylsilane and 7 mL TFA. The reaction mixture was capped and allowed to stir at room temperature for 1 h. LC-MS analysis confirmed complete consumption of compound 8.3b. The reaction mixture was concentrated to afford 6-cyclohexyl-5-[2-(2,4-dimethyl-thiazol-5-yl)-quinolin-6-yl]-4H-thieno[3,2-b]pyrrole-2-carboxylic acid methyl ester 8.4b. The crude residue was dried on vacuum and used without further purification. MS: 502.1 (M+H⁺).

### Step 2. 6-Cyclohexyl-5-[2-(2,4-dimethyl-thiazol-5-yl)-quinolin-6-yl]-4-(2-morpholin-4-yl-2-oxo-ethyl) -4H-thieno[3,2-b]pyrrole-2-carboxylic acid (compound 189)

A reaction vessel was charged with 361 mg (0.72 mmol) compound 8.4b and dissolved with 20 mL DMF. 58 mg (1.44 mmol, 2 eq) NaH (60% in mineral oil) was then added and the reaction mixture was allowed to stir at room temperature. After 15 min 167 μL (1.44 mmol, 2 eq) of 2-chloro-1-morpholin-4-yl-ethanone was added in 1 portion and the reaction mixture was allowed to continue stirring at room temperature. After 60 min, HPLC and LC-MS analysis confirmed complete consumption of compound 8.4b. The reaction mixture was quenched by adding 0.5 mL H₂O, poured into a 50 mL flask and concentrated. Cold H₂O was then added to the crude residue to precipitate the methyl ester as a dark powder. The solids were collected by centrifuge and washed once more with H₂O. The methyl ester was then transferred to a reaction vial and dissolved with 6 mL THF, 2 mL MeOH and 2 mL LiOH (1M, aq.). The reaction mixture was then heated to 50° C. and carefully monitored by HPLC and LC-MS analysis. Upon complete conversion, the reaction mixture was neutralized with 1 mL HCl (2M, aq.) and concentrated. The crude residue was then dissolved with DMF and acidified with TFA. The mixture was then filtered and purified by reverse-phase HPLC to afford 178 mg (39%) of 6-cyclohexyl-5-[2-(2,4-dimethyl-thiazol-5-yl)-quinolin-6-yl]-4-(2-morpholin-4-yl-2-oxo-ethyl)-4H-thieno[3,2-b]pyrrole-2-carboxylic acid (Compound 189) as an orange powder. MS: 615.2 (M+H⁺); ¹H NMR (DMSO-d6): δ (ppm) 8.53 (d, J=8.3, 1H), 8.08 (d, J=8.6, 1H), 7.96-7.93 (m, 2H), 7.86 (s, 1H), 7.68 (dd, J=8.6, 1.8, 1H), 5.00 (s, 2H), 3.51-3.36 (m, 8H), 2.76 (s, 3H), 2.71 (s, 3H), 1.86-1.20 (m, 10H).

### Example 9

### 6-Cyclohexyl-5-[2-(2,4-dimethyl-thiazol-5-yl)-quinolin-6-yl]-4-(2-oxo-2-thiomorpholin-4-yl-ethyl)-4H-thieno[3,2-b]pyrrole-2-carboxylic acid (compounds 44 and 190)

### Step 1. 4-tert-Butoxycarbonylmethyl-6-cyclohexyl-5-[2-(2,4-dimethyl-thiazol-5-yl)-quinolin-6-yl]-4H-thieno[3,2-b]pyrrole-2-carboxylic acid methyl ester (9.1)

A reaction vessel was charged with 191 mg (0.38 mmol) compound 8.4b (Example 8, Step 1) and dissolved with 15 mL DMF. 30 mg (0.76 mmol, 2 eq) NaH (60% in mineral oil) was then added and the reaction mixture was allowed to stir at room temperature. After 15 min 112 μL (0.76 mmol, 2 eq) of 2-tert-butylbromoacetate was added in 1 portion and the reaction mixture was allowed to continue stirring at room temperature. The reaction was monitored by HPLC and LC-MS analysis. Upon complete conversion of compound 8.4b, the reaction mixture was quenched by adding 0.5 mL H₂O, poured into a 50 mL flask and concentrated. Cold H₂O was then added to the crude residue to precipitate the methyl ester as a dark powder. The solids were collected by centrifuge and washed once more with H₂O. Crude 4-tert-butoxycarbonylmethyl-6-cyclohexyl-5-[2-(2,4-dimethyl-thiazol-5-yl)-quinolin-6-yl]-4H-thieno[3,2-b]pyrrole-2-carboxylic acid methyl ester 9.1 was then dried under vacuum and used without further purification. MS: 616.1 (M+H⁺).

### Step 2. 4-Carboxymethyl-6-cyclohexyl-5-[2-(2,4-dimethyl-thiazol-5-yl)-quinolin-6-yl]-4H-thieno[3,2-b]pyrrole-2-carboxylic acid methyl ester (9.2)

A 50 mL flask was charged with 234 mg (0.38 mmol) compound 9.1 and dissolved with 5 mL 4M HCl in dioxane. 250 μL (5% v/v) anisole was then added and the reaction mixture was allowed to stir at room temperature. After HPLC and LC-MS analysis indicated complete conversion of compound 9.1, the reaction mixture was concentrated. Crude 4-carboxymethyl-6-cyclohexyl-5-[2-(2,4-dimethyl-thiazol-5-yl)-quinolin-6-yl]-4H-thieno[3,2-b]pyrrole-2-carboxylic acid methyl ester 9.2 was then dried briefly under vacuum and used without further purification. MS: 560.1 (M+H⁺).

### Step 3. 6-Cyclohexyl-5-[2-(2,4-dimethyl-thiazol-5-yl)-quinolin-6-yl]-4-(2-oxo-2-thiomorpholin-4-yl-ethyl)-4H-thieno[3,2-b]pyrrole-2-carboxylic acid (compound 190)

A reaction vessel was charged with 107 mg (0.19 mmol) compound 9.2 and dissolved with 3 mL DMF. 86 mg (0.23 mmol, 1.2 eq) HBTU and 73 μL (0.42 mmol, 2.2 eq) DIEA was then added and the reaction mixture was allowed to stir at room temperature. After 15 min 24 μL (0.24 mmol, 1.25 eq) of thiomorpholine was added in 1 portion and the reaction mixture was allowed to continue stirring at 35° C. After HPLC and LC-MS analysis confirmed complete consumption of compound 9.2, the reaction mixture was concentration by speed-vacuum. Cold H₂O was then added to the crude residue to precipitate the methyl ester. The solids were collected by centrifuge and washed once more with H₂O. The methyl ester was then transferred to a reaction vial and dissolved with 3 mL THF, 1 mL MeOH and 1 mL LiOH (1M, aq.). The reaction mixture was then heated to 50° C. and carefully monitored by HPLC and LC-MS analysis. Upon complete conversion, the reaction mixture was neutralized with 0.5 mL HCl (2M, aq.) and concentrated. The crude residue was then dissolved with DMF and acidified with TFA. The mixture was then filtered and purified by reverse-phase HPLC to afford 24 mg (20%) of 6-cyclohexyl-5-[2-(2,4-dimethyl-thiazol-5-yl) -quinolin-6-yl]-4-(2-oxo-2-thiomorpholin-4-yl-ethyl)-4H-thieno[3,2-b]pyrrole-2-carboxylic acid (compound 190) as an orange powder. MS: 631.2 (M+H⁺); ¹H NMR (DMSO-d6): δ (ppm) 8.53 (d, J=8.8, 1H), 8.08 (d, J=8.8, 1H), 7.95-7.87 (m, 3H), 7.68 (dd, J=8.5, 2.0, 1H), 5.00 (s, 2H), 3.67-3.57 (m, 4H), 2.76 (s, 3H), 2.71 (s, 3H), 2.47-2.35 (m, 4H), 1.86-1.21 (m, 10H).

### Example 10

### 6-Cyclohexyl-5-[2-(2,4-dimethyl-thiazol-5-yl)-quinolin-6-yl]-4-(2-oxo-2-piperidin-1-yl-ethyl) -4H-thieno[3,2-b]pyrrole-2-carboxylic acid (compounds 35 and 191)

A reaction vessel was charged with 107 mg (0.19 mmol) compound 9.2 (Example 9, Step 2) and dissolved with 3 mL DMF. 86 mg (0.23 mmol, 1.2 eq) HBTU and 73 μL (0.42 mmol, 2.2 eq) DIEA was then added and the reaction mixture was allowed to stir at room temperature. After 15 min 24 μL (0.24 mmol, 1.25 eq) of piperidine was added in 1 portion and the reaction mixture was allowed to continue stirring at 35° C. After HPLC and LC-MS analysis confirmed complete consumption of compound 9.2, the reaction mixture was concentration by speed-vacuum. Cold H₂O was then added to the crude residue to precipitate the methyl ester. The solids were collected by centrifuge and washed once more with H₂O. The methyl ester was then transferred to a reaction vial and dissolved with 3 mL THF, 1 mL MeOH and 1 mL LiOH (1M, aq.). The reaction mixture was then heated to 50° C. and carefully monitored by HPLC and LC-MS analysis. Upon complete conversion, the reaction mixture was neutralized with 0.5 mL HCl (2M, aq.) and concentrated. The crude residue was then dissolved with DMF and acidified with TFA. The mixture was then filtered and purified by reverse-phase HPLC to afford 24 mg (21%) of 6-cyclohexyl-5-[2-(2,4-dimethyl-thiazol-5-yl)-quinolin-6-yl]-4-(2-oxo-2-piperidin-1-yl-ethyl)-4H-thieno[3,2-b]pyrrole-2-carboxylic acid (compound 191) as an orange powder. MS: 613.2 (M+H⁺); ¹H NMR (DMSO-d6): δ (ppm) 8.52 (d, J=8.5, 1H), 8.07 (d, J=8.5, 1H), 7.95-7.92 (m, 2H), 7.82 (s, 1H), 7.69 (d, J=8.2, 1H), 5.96 (s, 2H), 3.38-3.26 (m, 4H), 2.76 (s, 3H), 2.71 (s, 3H), 1.86-1.24 (m, 16H).

Prophetic compounds 1-34, 36-43, and 45-185 in Table I can be similarly prepared according to the general synthetic methods and examples described above.

### Biological Examples

### Biological Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application Ser. No. 60/004,383, filed on September 1995, described an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1 (Supp 4) 18-24.

Screens that measure reductions in kinase activity from HCV drugs were disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs were disclosed in U.S. Pat. No. 5,861,267 to Su et al., U.S. Pat. No. 5,739,002 to De Francesco et al., and U.S. Pat. No. 5,597,691 to Houghton et al.

### Biological Example 2

### Replicon Assay

A cell line, ET (Huh-lucubineo-ET) was used for screening of compounds for inhibiting HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T12801; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They are all available through Life Technologies (Bethesda, Md.). The cells were plated at 0.5-1.0×10⁴ cells/well in the 96 well plates and incubated for 24 hrs before adding test compound. The compounds were added to the cells to achieve a final concentration of 0.1 nM to 50 μm and a final DMSO concentration of 0.5%. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data was plotted relative to no compound control. Under the same condition, cytotoxicity of the compounds were determined using cell proliferation reagent, WST-1 (Roche, Germany). The compounds showing antiviral activities, but no significant cytotoxicities were chosen to determine EC₅₀ and TC₅₀, the effective concentration and toxic concentration at which 50% of the maximum inhibition is observed. For these determinations, a 10 point, 2-fold serial dilution for each compound was used, which spans a concentration range of 1000 fold. EC₅₀ and similarly the TC₅₀ values were calculated by fitting % inhibition at each concentration to the following equation:
% inhibition=100%/[(EC₅₀/[I])^{b}+1]
where b is Hill's coefficient.

In some aspects, the compounds of Formula (I) will have an EC₅₀ of equal to or less than 50 μM when tested according to the assay of Example 2. In other aspects the EC₅₀ is equal to or less than 10 μM. In still other aspects the EC₅₀ is equal to or less than 5 μM.

When tested at 6.25 μM, compounds 186-191 where found respectively to have the following % inhibition values: 75, 63, 99, 100, 98, and 97.

### Biological Example 3

### Cloning and Expression of Recombinant HCV-NS5b

The coding sequence of NS5b protein was cloned by PCR from pFKI₃₈₉luc/NS3-3′/ET as described by Lohmann, V., et al. (1999) Science 285, 110-113 using the primers shown on page 266 of WO 2005/012288.

The cloned fragment is missing the C terminus 21 amino acid residues. The cloned fragment was inserted into an IPTG-inducible expression plasmid that provides an epitope tag (His)₆ at the carboxy terminus of the protein.

The recombinant enzyme was expressed in XL-1 cells and after induction of expression, the protein was purified using affinity chromatography on a nickel-NTA column. Storage condition is 10 mM Tris-HCl pH 7.5, 50 mM NaCl, 0.1 mM EDTA, 1 mM DTT, 20% glycerol at −20° C.

### Biological Example 4

### HCV-NS5b Enzyme Assay

The polymerase activity was assayed by measuring incorporation of radiolabeled UTP into a RNA product using a biotinylated, heteropolymeric template, which includes a portion of the HCV genome. Typically, the assay mixture (50 μL) contains 10 mM Tris-HCl (pH 7.5), 5 mM MgCl₂, 0.2 mM EDTA, 10 mM KCl, 1 unit/μL RNAsin, 1 mM DTT, 10 μM each of NTP, including [³H]-UTP, and 10 ng/μL heteropolymeric template. Test compounds were initially dissolved in 100% DMSO and further diluted in aqueous buffer containing 5% DMSO. Typically, compounds were tested at concentrations between 1 nM and 100 μM. Reactions were started with addition of enzyme and allowed to continue at 37° C. for 2 hours. Reactions were quenched with 8 μL of 100 mM EDTA and reaction mixtures (30 μL) were transferred to streptavidin-coated scintillation proximity microtiter plates (FlashPlates) and incubated at 4° C. overnight. Incorporation of radioactivity was determined by scintillation counting.

### Formulation Examples

The following are representative pharmaceutical formulations containing a compound of Formula (I).

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. to 100 | mL |

distilled water q.s. to 100 mL

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

1. A compound of Formula (I) or a pharmaceutically acceptable salt or tautomer thereof: 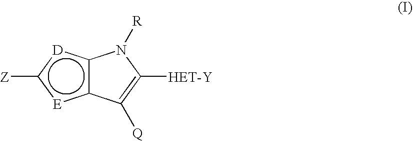
wherein:
Y is selected from the group consisting of aryl, heteroaryl, substituted aryl, and substituted heteroaryl;
HET is selected from the group consisting of a 6-membered arylene ring, a 6-membered heteroarylene ring containing 1, 2, or 3 heteroatoms selected from N, O, or S, and a bicyclic ring having the formula 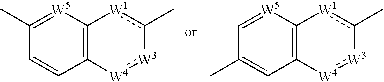
wherein HET is optionally substituted with (X)ₜ, X is selected from the group consisting of alkyl, substituted alkyl, alkoxy, substituted alkoxy, amino, substituted amino, halo, hydroxy, and nitro; t is an integer equal to 0, 1 or 2; W¹, W⁴, and W⁵ are independently N or CH; W³ is N, CH, or is a bond provided that no more than one nitrogen in the bicyclic ring is optionally oxidized to form an N-oxide; and each dashed line independently represents a single or double bond between the two adjoining atoms, provided that when one of dashed lines is a single bond, the adjoining atoms are each substituted with 1 or 2 hydrogen atoms to satisfy its valency;
one of D or E is C—R^{a} and the other of D or E is S;
R^{a} and R are independently selected from the group consisting of hydrogen, alkyl and substituted alkyl;
Q is selected from the group consisting of cycloalkyl, substituted cycloalkyl, cycloalkenyl, substituted cycloalkenyl, heterocyclic, substituted heterocyclic, aryl, substituted aryl, heteroaryl, and substituted heteroaryl; and
Z is selected from the group consisting of
(a) carboxy and carboxy ester;
(b) —C(X⁴)NR⁸R⁹, wherein X⁴ is ═O, ═NH, or ═N-alkyl, R⁸ and R⁹ are independently selected from the group consisting of hydrogen, alkyl, substituted alkyl alkenyl, substituted alkenyl, alkynyl, substituted alkynyl, aryl, substituted aryl, heteroaryl, substituted heteroaryl, heterocyclic and substituted heterocyclic or, alternatively, R⁸ and R⁹ together with the nitrogen atom pendent thereto, form a heterocyclic, a substituted heterocyclic, a heteroaryl or a substituted heteroaryl ring group;
(c) —C(X³)NR²¹S(O)₂R⁴, wherein X³ is selected from ═O, ═NR²⁴, and ═S, wherein R²⁴ is hydrogen, alkyl, or substituted alkyl; R⁴ is selected from alkyl, substituted alkyl, aryl, substituted aryl, heteroaryl, substituted heteroaryl, heterocyclic, substituted heterocyclic, and NR²²R²³ wherein R²¹, R²² and R²³ are independently hydrogen, alkyl, substituted alkyl cycloalkyl, or substituted cycloalkyl; or alternatively, R²¹ and R²² or R²² and R²³ together with the atoms bound thereto join together to form an optionally substituted heterocyclic group;
(d) —C(X²)—N(R³)CR²R^{2′}C(═O)R¹, wherein X² is selected from ═O, ═S, and ═NR¹¹, where R¹¹ is hydrogen or alkyl R¹ is selected from —OR⁷ and —NR⁸R⁹ where R⁷ is selected from hydrogen, alkyl substituted alkyl alkenyl, substituted alkenyl, alkynyl, substituted alkynyl, aryl, substituted aryl, heteroaryl, substituted heteroaryl, heterocyclic and substituted heterocyclic; R⁸ and R⁹ are as defined above;
R² and R^{2′} are independently selected from hydrogen, alkyl, substituted alkyl alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic;
or, alternatively, R² and R^{2′} as defined are taken together with the carbon atom pendent thereto to form a cycloalkyl, substituted cycloalkyl, heterocyclic or substituted heterocyclic group,
or, still further alternatively, one of R² or R^{2′} is hydrogen, alkyl or substituted alkyl, and the other is joined, together with the carbon atom pendent thereto, with either the R⁷ and the oxygen atom pendent thereto or R⁸ and the nitrogen atom pendent thereto to form a heterocyclic or substituted heterocyclic group;
R³ is selected from hydrogen and alkyl or, when R² and R^{2′} are not taken together to form a ring and when R² or R^{2′} and R⁷ or R⁸ are not joined to form a heterocyclic or substituted heterocyclic group, then R³, together with the nitrogen atom pendent thereto, may be taken together with one of R² and R^{2′} to form a heterocyclic or substituted heterocyclic ring group;
(e) —C(X²)—N(R³)CR²⁵R²⁶R²⁷, wherein X² and R³ are defined above, and R²⁵, R²⁶ and R²⁷ are independently selected from the group consisting of alkyl, substituted alkyl, aryl, substituted aryl, heterocyclic, substituted heterocyclic, heteroaryl and substituted heteroaryl, or R²⁵ and R²⁶ together with the carbon atom pendent thereto form a cycloalkyl, substituted cycloalkyl, heterocyclic or substituted heterocyclic group; and
(f) a carboxylic acid isostere wherein said isostere is not as defined in (a)-(e).

2. A compound of claim 1 wherein Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized.

3. A compound of claim 2 wherein Y is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphen-2-yl, 4-methoxybiphen-2-yl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

4. A compound of claim 2 wherein said substituted phenyl is substituted with one to three substitutents selected from the group consisting of halo, heteroaryl, hydroxy, nitro, cyano, alkyl, substituted alkyl, alkenyl, alkoxy, substituted alkoxy, acyl, acylamino, aminoacyl, amino, substituted amino, carboxy, and carboxy ester.

5. A compound of claim 2 wherein Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl.

6. A compound of claim 5 wherein Y is substituted with one to three substitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester.

7. A compound of claim 6 wherein Y is 2,4-dimethylthiazol-5-yl.

8. A compound of claim 1 wherein Q is cycloalkyl or cycloalkenyl.

9. A compound of claim 8 wherein Q is cyclohexyl or cyclohexenyl.

10. A compound of claim 1 wherein Z is carboxy, carboxy ester, carboxylic acid isostere, —C(O)NR⁸R⁹, or —C(O)NHS(O)₂R⁴, wherein R⁸ and R⁹ are as defined in claim 1 and R⁴ is alkyl or aryl.

11. A compound of claim 10 wherein Z is carboxy, methyl carboxylate, ethyl carboxylate, 6-(β-D-glucuronic acid) ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl.

12. A compound of claim 11 wherein Z is carboxy.

13. A compound of claim 1 wherein D is CH and E is S.

14. A compound of claim 1 wherein R is substituted alkyl, wherein said substituted alkyl is selected from the group consisting of aminoalkyl, substituted aminoalkyl, arylalkyl, substituted arylalkyl, heteroarylalkyl, substituted heteroarylalkyl, heterocyclylalkyl, substituted heterocyclylalkyl, —CH₂COOH, and —CH₂CONR¹²R¹³, wherein R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms.

15. A compound of claim 1 wherein R is —CH₂CONR¹²R¹³ and at least one of R¹² or R¹³ is alkyl, substituted alkyl, or heteroaryl.

16. A compound of claim 15 wherein at least one of R¹² or R¹³ is methyl, carboxymethyl, 2-hydroxyethyl, 2-morpholin-4-ylethyl, or tetrazoyl-5-yl.

17. A compound of claim 14 wherein R is —CH₂CONR¹²R¹³ and R¹² and R¹³ and the nitrogen atom to which they are attached form a substituted or unsubstituted heterocyclic ring.

18. A compound of claim 17 wherein R¹² and R¹³ and the nitrogen atom to which they are attached form a substituted or unsubstituted morpholino, substituted or unsubstituted piperidinyl, or a substituted or unsubstituted pyrrolidinyl ring.

19. A compound of claim 18 wherein said substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

20. The compound of claim 14 wherein R is selected from N,N-dimethylamino-carbonylmethyl, [N-(4-hydroxy-1,1-dioxidotetrahydro-3-thienyl)amino]-carbonylmethyl, (cyclopropylmethylamino)-carbonylmethyl, (prop-2-yn-1-ylamino)-carbonylmethyl, (2-(morpholino)eth-1-ylamino)-carbonylmethyl, (phenylsulfonylamino)-carbonylmethyl, [N-benzylamino]-carbonylmethyl, (N-(4-methylsulfonyl-benzyl)amino)-carbonylmethyl, (tryptophanyl)-carbonylmethyl, (tyrosine)-carbonylmethyl, (N-(1-carboxyprop-1-ylamino)-carbonylmethyl, (N-(2-carboxyeth-1-yl)-amino)-carbonylmethyl, (N-(4-carboxybenzyl)-amino)-carbonylmethyl, N-[3-(N′-(4-(acrylic acid)-phenyl)carboxamido)pyrrolidin-3-yl]amino-carbonylmethyl, N-[4-(N′-(4-(acrylic acid)-phenyl)carboxamido)piperidin-4-yl]amino-carbonylmethyl, [2-(N,N-dimethylamino)eth-1-ylamino]-carbonylmethyl, [(1-(5-methyl-4H-1,2,4-triazol-3-yl)ethyl)amino]-carbonylmethyl, (1-methyl-1-[N-(1-methyl-2-carboxy-1H-indol-5-yl)aminocarbonyl]eth-1-ylamino-carbonylmethyl, [N-(1-methylpyrrolidin-3-yl-ethyl)-amino]-carbonylmethyl, (1-methyl-1-[N-(4-(acrylic acid)phenyl)aminocarbonyl]eth-1-ylamino-carbonylmethyl, (1-methyl-1-[N-(4-(2-carboxy-furan-5-yl)phenyl)aminocarbonyl]eth-1-ylamino-carbonylmethyl, (1-methyl-1-[N-(4-(4-carboxy-thiazol-2-yl)phenyl)aminocarbonyl]eth-1-ylamino-carbonylmethyl, (2-(4-methylpiperazin-1-yl)eth-1-ylamino)-carbonylmethyl, [(1-methylpyrrolidin-3-yl)methylamino]-carbonylmethyl, [N-(1-methylpiperidin-3-yl-methyl)-amino]-carbonylmethyl, (1-piperidin-1-ylcyclopentyl)methylamino]-carbonylmethyl, (1-(acetyl)-pyrrolidin-2-ylmethyl)amino)-carbonylmethyl, [(2-(N,N-dimethylamino)-carbonyl)methylamino]-carbonylmethyl, [N-(1,1-dioxidotetrahydro-3-thienyl)methylamino]-carbonylmethyl, (N-methyl-N-cyclohexyl-amino)-carbonylmethyl, (N-methyl-N-carboxymethyl-amino)-carbonylmethyl, [N-methyl-N-benzyl-amino]-carbonylmethyl, (N-methyl-N—(N′,N′-dimethylaminoacetyl)-amino)-carbonylmethyl, [N-methyl-N-phenyl-amino]-carbonylmethyl, (N-methyl-N-isopropyl-amino)-carbonylmethyl, (N-methyl-N—(N′-methylpiperidin-4-yl)amino)-carbonylmethyl, [N-methyl-N-(1-methylpiperidin-4-yl)amino]-carbonylmethyl, [N-methyl-N-(1-methylpiperidin-4-yl-methyl)-amino]-carbonylmethyl, [N-methyl-N-(1-methylpiperidin-3-yl-methyl)-amino]-carbonylmethyl, [N-methyl-N-(1-methylpyrazin-2-yl-methyl)-amino]-carbonylmethyl, [N-methyl-N-(5-methyl-1H-imidazol-2-ylmethyl)-amino]-carbonylmethyl, (N-methyl-N-[2-(hydroxy)eth-1-yl]amino)-carbonylmethyl, (N-methyl-N-[2-(N′,N′-dimethylamino)eth-1-yl]amino)-carbonylmethy, N-methyl-N-[2-(N′,N′-diethylamino)eth-1-yl]amino)-carbonylmethyl, (N-methyl-N-[2-(pyridin-2-yl)eth-1-yl]amino)-carbonylmethyl, (N-methyl-N-[2-(pyridin-4-yl)eth-1-yl]amino)-carbonylmethyl, [N-methyl-N-(1-(1,3-thiazol-2-yl)ethyl)-amino]-carbonylmethyl, (N-methyl-N-[3-(N′,N′-dimethylamino)prop-1-yl]amino)-carbonylmethyl, (N-methyl-N-(1-carboxy-2-methylprop-1-yl)-amino)-carbonylmethyl, (N-ethyl-N-propyl-amino)-carbonylmethyl, (N-ethyl-N-[2-(methoxy)eth-1-yl]amino)-carbonylmethyl, (N-ethyl-N-[2-(N′,N′-diethylamino)eth-1-yl]amino)-carbonylmethyl, [7-methyl-2,7-diazaspiro[4.4]non-2-yl]-carbonylmethyl, (5-methyl-2,5-diazabicyclo[2.2.1]heptyl-2-yl)-carbonylmethyl, (4-methyl-1,4-diazepan-1-yl)-carbonylmethyl, (piperidinyl)-carbonylmethyl, (4-carboxy-piperidinyl)-carbonylmethyl, (3-carboxypiperidinyl)-carbonylmethyl, (4-hydroxypiperidinyl)-carbonylmethyl, (4-(2-hydroxyeth-1-yl)piperidin-1-yl)-carbonylmethyl, [4-(N,N-dimethylamino) -piperidin-1-yl]-carbonylmethyl, (3-(N,N-dimethylamino) -methylpiperidin-1-yl)-carbonylmethyl, (2-(2-(N,N-dimethylamino)-eth-1-yl)piperidin-1-yl)-carbonylmethyl, [4-(4-methyl-4H-1,2,4-triazol-3-yl)piperidin-1-yl]-carbonylmethyl, (4-pyrrolidinyl-piperidinyl)-carbonylmethyl, (3-pyrrolidinyl-piperidinyl)-carbonylmethyl, [4-(N,N-diethylamino)-piperidin-1-yl]-carbonylmethyl, (4-(azetidin-1-yl)-piperidin-1-yl)-carbonylmethyl, (4-(piperidin-1-yl) -piperidin-1-yl)-carbonylmethyl, (hexahydropyrrolo[1,2-a]pyrazin-2(1H)-yl)-carbonylmethyl, [(2-(N,N-dimethylamino)-methyl)morpholino]-carbonylmethyl, (3,5-dimethylmorpholino)-carbonylmethyl, (thiomorpholino)-carbonylmethyl, morpholino-carbonylmethyl, (pyrrolidinyl)-carbonylmethyl, (2-carboxy-pyrrolidin-1-yl)-carbonylmethyl, (2-(carboxy)-4-hydroxy-pyrrolidin-1-yl)-carbonylmethyl, (2-carboxamide-pyrrolidin-1-yl)-carbonylmethyl, (2-(N,N-dimethylaminocarbonyl) -pyrrolidin-1-yl)-carbonylmethyl, (3-(N′,N′-dimethylamino) -pyrrolidin-1-yl)-carbonylmethyl, (3-(N′,N′-diethylamino)-pyrrolidin-1-yl)-carbonylmethyl, (3-(pyridin-3-yl) -pyrrolidin-1-yl)-carbonylmethyl, (2-pyidin-4-ylpyrrolidin-1-yl)-carbonylmethyl, piperazin-1-yl-carbonylmethyl, (4-methylpiperazinyl)-carbonylmethyl, (4-(carboxymethyl)-piperazin-1-yl)-carbonylmethyl, (4-(2-hydroxyeth-1-yl)piperazin-1-yl)-carbonylmethyl, (4-(isopropyl) piperazin-1-yl)-carbony1-yl)-carbonylmethyl, (4-(N′,N′-dimethylaminoacetyl)-piperazin-1-yl)-carbonylmethyl, lmethyl, (4-(2-methoxyeth-1-yl)piperazin-1-yl)-carbonylmethyl, (4-(ethyl)piperazin-and (4-(6-methoxypyridin-2-yl)piperazin-1-yl)-carbonylmethyl.

21. A compound of claim 1 wherein t is 0.

22. A compound of claim 1 wherein t is 1 and X is amino, nitro, methyl or halo.

23. A compound according to claim 1 wherein HET is 1,4-phenylene optionally substituted with (X)ₜ where X and t are as defined in claim 1.

24. A compound of claim 23 wherein t is 0.

25. A compound of claim 1 wherein HET is 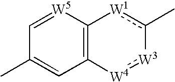
optionally substituted with (X)ₜ where X, t, W¹, W³, W⁴, and W⁵ are as defined in claim 1.

26. A compound of claim 25 wherein W¹ is nitrogen.

27. A compound of claim 26 where HET is selected from the group consisting of 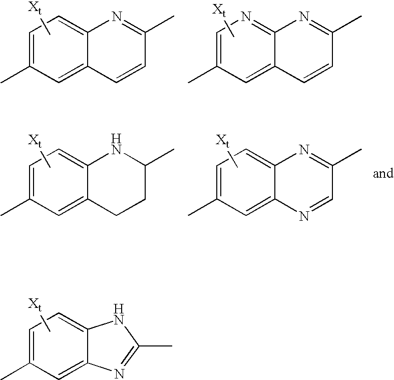
where X and t are as defined in claim 1.

28. A compound having Formula (Ia) or a pharmaceutically acceptable salt or tautomer thereof: 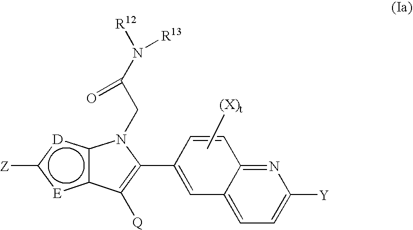
wherein:
Y is selected from the group consisting of substituted aryl and substituted heteroaryl;
X is independently selected from the group consisting of amino, nitro, alkyl, haloalkyl, and halo;
t is an integer equal to 0, 1 or 2;
Q is selected from the group consisting of cyclohexyl and cyclopentyl;
R¹² and R¹³ are independently selected from hydrogen, alkyl, substituted alkyl, alkenyl, substituted alkenyl, alkynyl, substituted alkynyl, alkoxy, substituted alkoxy, —(CH₂)₀₋₃R¹⁶, and —NR¹⁷R¹⁸, or R¹² and R¹³ and the nitrogen atom to which they are attached form a substituted or unsubstituted heterocyclic ring provided that both R¹² and R¹³ are not both hydrogen; wherein R¹⁶ is aryl, heteroaryl, or heterocyclic; and R¹⁷ and R¹⁸ are independently hydrogen or alkyl or R¹⁷ and R¹⁸ together with the nitrogen atom to which they are attached join to form a heterocyclic ring with 4 to 7 ring atoms;
one of A or B is C—R^{a} and the other of A or B is S;
R^{a} is selected from the group consisting of hydrogen, alkyl, and substituted alkyl; and
Z is selected from the group consisting of carboxy, carboxy ester, and a carboxylic acid isostere.

29. A compound of claim 28 wherein Y is selected from the group consisting of substituted biphenyl, substituted phenyl, substituted 6-membered heteroaryl ring optionally fused to a phenyl ring and having one, two, or three heteroatoms independently selected from the group consisting of N, O, or S wherein the heteroatoms N or S are optionally oxidized, and substituted 5-membered heteroaryl ring optionally fused to a phenyl ring and having one, two, or three heteroatoms independently selected from the group consisting of N, O, or S wherein the heteroatoms N or S are optionally oxidized.

30. A compound of claim 29 wherein Y is selected from the group consisting of 4′-chloro-4-methoxybiphen-2-yl, biphen-2-yl, biphen-4-yl, 4-amino-4′-chlorobiphen-2-yl, 4′-aminomethyl-4-methoxybiphen-2-yl, 4-carbamoyl-4′-methoxybiphen-2-yl, 4-carbamoyl-4′-fluorobiphen-2-yl, 4-carbamoyl-4′-methoxybiphen-2-yl, 4-carbamoyl-4′-nitrobiphen-2-yl, 4-(carbamoylmethyl-carbamoyl)biphen-2-yl, 4-(carbamoylmethylcarbamoyl)-4′-chlorobiphen-2-yl, 4-carboxy-4′-chlorobiphen-2-yl, 3-carboxy-4′-methoxybiphen-2-yl, 4-carboxy-4′-methoxybiphen-2-yl, 4′-carboxy-4-(pyrrolidin-1-ylcarbonyl)biphen-2-yl, 4-carboxymethoxybiphen-2-yl, 4-carboxymethoxy-4′-chlorobiphen-2-yl, 4′-chlorobiphen-2-yl, 4′-chloro-4-chlorobiphen-2-yl, 4′-chloro-4-(dimethylaminoethylcarbamoylbiphen-2-yl, 4′-chloro-4-(2-ethoxyethoxy)biphen-2-yl, 3′-chloro-4′-fluoro-4-methoxybiphen-2-yl, 4′-chloro-4-fluorobiphen-2-yl, 4′-chloro-4-hydroxybiphen-2-yl, 3′-chloro-4-methoxybiphen-2-yl, 4′-chloro-4-methylcarbamoylbiphen-2-yl, 4′-chloro-4-(2-methoxyethoxy)biphen-2-yl, 4′-chloro-4-nitrobiphen-2-yl, 4′-chloro-4-(2-oxo-2-pyrrolidin-1-ylethoxy)biphen-2-yl, 4′-chloro-4-(pyrrolidin-1-ylcarbonyl)biphen-2-yl, 4′-chloro-4-(3-pyrrolidin-1-ylpropoxy)biphen-2-yl, 4′-cyano-4-methoxybiphen-2-yl, 3′,4′-dichloro-4-methoxybiphen-2-yl, 4,4′-dimethoxybiphen-2-yl, 3′,4′-dimethoxy-4-(pyrrolidin-1-ylcarbonyl)biphen-2-yl, 4′-dimethylamino-4-methoxybiphen-2-yl, 4-(2-dimethylaminoethylcarbamoyl)biphen-2-yl, 4′-ethoxy-4-methoxybiphen-2-yl, 4′-fluoro-4-methoxybiphen-2-yl, 4-hydroxybiphen-2-yl, 4-methoxybiphen-2-yl, 4-methoxy-4′-hydroxybiphen-2-yl, 4-(2-methoxyethoxy)biphen-2-yl, 4-methoxy-4′-methylbiphen-2-yl, 4-methoxy-3′-nitrobiphen-2-yl, 4-methoxy-4′-nitrobiphen-2-yl, 4-methylcarbamoylbiphen-2-yl, 3′-methyl-4-methoxybiphen-2-yl, 4′-nitro-4-(pyrrolidin-1-ylcarbonyl)biphen-2-yl, 4-(2-oxo-2-pyrrolidin-1-ylethoxy)biphen-2-yl, 4-(3-pyrrolidin-1-ylpropoxy)biphen-2-yl, and 4′-trifluoromethyl-4-methoxybiphen-2-yl.

31. A compound of claim 29 wherein said substituted phenyl is substituted with one to three substitutents selected from the group consisting of halo, heteroaryl, hydroxy, nitro, cyano, alkyl, substituted alkyl, alkenyl, alkoxy, substituted alkoxy, acyl, acylamino, aminoacyl, amino, substituted amino, carboxy, and carboxy ester.

32. A compound of claim 29 wherein Y is selected from the group consisting of substituted quinolyl, substituted benzofuryl, substituted thiazolyl, substituted furyl, substituted thienyl, substituted pyridinyl, substituted pyrazinyl, substituted oxazolyl, substituted isoxazolyl, substituted pyrrolyl, substituted imidazolyl, substituted pyrrolidinyl, substituted pyrazolyl, substituted isothiazolyl, substituted 1,2,3-oxadiazolyl, substituted 1,2,3-triazolyl, substituted 1,3,4-thiadiazolyl, substituted pyrimidinyl, substituted 1,3,5-triazinyl, substituted indolizinyl, substituted indolyl, substituted isoindolyl, substituted indazolyl, substituted benzothienyl, substituted benzthiazolyl, substituted purinyl, substituted quinolizinyl, substituted quinolinyl, substituted isoquinolinyl, substituted cinnolinyl, substituted phthalazinyl, substituted quinazolinyl, substituted quinoxalinyl, substituted 1,8-naphthyridinyl, and substituted pteridinyl.

33. A compound of claim 32 wherein Y is substituted with one to three substitutents independently selected from the group consisting of alkyl, haloalkyl, halo, hydroxy, nitro, cyano, alkoxy, substituted alkoxy, acyl, acylamino, aminoacyl, amino, substituted amino, carboxy, and carboxy ester.

34. A compound of claim 33 wherein Y is 2,4-dimethylthiazol-5-yl.

35. A compound of claim 28 wherein Q is cyclohexyl or cyclohexenyl.

36. A compound of claim 35 wherein Z is methyl carboxylate, ethyl carboxylate, 6-(β-D-glucuronic acid) ester, 1H-tetrazol-5-yl, 5-oxo-4,5-dihydro-1,2,4-oxadiazol-3-yl, N-2-cyano-ethylamide, N-2-(1H-tetrazol-5-yl)ethylamide, methylsulfonylaminocarbonyl, trifluoromethylsulfonylaminocarbonyl, or phenylsulfonylaminocarbonyl.

37. A compound of claim 36 wherein Z is carboxy.

38. A compound of claim 28 wherein at least one of R¹² or R¹³ is alkyl, substituted alkyl, or heteroaryl.

39. A compound of claim 38 wherein at least one of R¹² or R¹³ is methyl, carboxymethyl, 2-hydroxyethyl, 2-morpholin-4-ylethyl, or tetrazoyl-5-yl.

40. A compound of claim 28 wherein R¹² and R¹³ and the nitrogen atom to which they are attached form a substituted or unsubstituted heterocyclic ring.

41. A compound of claim 40 wherein R¹² and R¹³ and the nitrogen atom to which they are attached form a substituted or unsubstituted morpholino, substituted or unsubstituted piperidinyl, or a substituted or unsubstituted pyrrolidinyl ring.

42. A compound of claim 41 wherein said substituted or unsubstituted morpholino, piperidinyl, or pyrrolidinyl ring is selected from the group consisting of morpholino, 4-pyrrolidin-1-yl-piperidinyl, piperidinyl, 4-hydroxypiperidinyl, 4-carboxypiperidinyl, 4-dimethylaminopiperidinyl, 4-diethylaminopiperidinyl, 2-methylpyrrolidinyl, 4-morpholin-4-yl-piperidinyl, 3,5-dimethyl-morpholin-4-yl, 4-methylpiperidinyl.

43. A compound of claim 28 wherein R¹² and R¹³ and the nitrogen atom to which they are attached together form a group selected from N,N-dimethylamino, N-(4-hydroxy-1,1-dioxidotetrahydro-3-thienyl)amino, cyclopropylmethylamino, prop-2-yn-1-ylamino, 2-(morpholino)eth-1-ylamino, phenylsulfonylamino, N-benzylamino, N-(4-methylsulfonyl-benzyl)amino, tryptophanyl, tyrosine, N-1-carboxyprop-1-ylamino, N-(2-carboxyeth-1-yl)-amino, N-(4-carboxybenzyl)-amino, N-[3-(N′-(4-(acrylic acid)-phenyl)carboxamido)pyrrolidin-3-yl]amino, N-[4-(N′-(4-(acrylic acid)-phenyl)carboxamido)piperidin-4-yl]amino, 2-(N,N-dimethylamino) eth-1-ylamino, (1-(5-methyl-4H-1,2,4-triazol-3-yl)ethyl)amino, 1-methyl-1-[N-(1-methyl-2-carboxy-1H-indol-5-yl)aminocarbonyl]eth-1-ylamino, N-(1-methylpyrrolidin-3-yl-ethyl)-amino, 1-methyl-1-[N-(4-(acrylic acid)phenyl)aminocarbonyl]eth-1-ylamino, 1-methyl-1-[N-(4-(2-carboxy-furan-5-yl)phenyl)aminocarbonyl]eth-1-ylamino, 1-methyl-1-[N-(4-(4-carboxy-thiazol-2-yl)phenyl)aminocarbonyl]eth-1-ylamino, 2-(4-methylpiperazin-1-yl)eth-1-ylamino, (1-methylpyrrolidin-3-yl)methylamino, N-(1-methylpiperidin-3-yl-methyl)-amino, (1-piperidin-1-ylcyclopentyl)methylamino, 1-(acetyl)-pyrrolidin-2-ylmethyl)amino, (2-(N,N-dimethylamino)-carbonyl)methylamino, N-(1,1-dioxidotetrahydro-3-thienyl)methylamino, N-methyl-N-cyclohexyl-amino, N-methyl-N-carboxymethyl-amino, N-methyl-N-benzyl-amino, N-methyl-N—(N′,N′-dimethylaminoacetyl)-amino, N-methyl-N-phenyl-amino, N-methyl-N-isopropyl-amino, N-methyl-N—(N′-methylpiperidin-4-yl)amino, N-methyl-N-(1-methylpiperidin-4-yl)amino, N-methyl-N-(1-methylpiperidin-4-yl-methyl)-amino, N-methyl-N-(1-methylpiperidin-3-yl-methyl)-amino, N-methyl-N-(1-methylpyrazin-2-yl-methyl)-amino, N-methyl-N-(5-methyl-1H-imidazol-2-ylmethyl)-amino, N-methyl-N-[2-(hydroxy)eth-1-yl]amino, N-methyl-N-[2-(N′,N′-dimethylamino) eth-1-yl]amino, N-methyl-N-[2-(N′,N′-diethylamino)eth-1-yl]amino, N-methyl-N-[2-(pyridin-2-yl)eth-1-yl]amino, N-methyl-N-[2-(pyridin-4-yl)eth-1-yl]amino, N-methyl-N-(1-(1,3-thiazol-2-yl)ethyl)-amino, N-methyl-N-[3-(N′,N′-dimethylamino)prop-1-yl]amino, N-methyl-N-(1-carboxy-2-methylprop-1-yl)-amino, N-ethyl-N-propyl-amino, N-ethyl-N-[2-(methoxy)eth-1-yl]amino, N-ethyl-N-[2-(N′,N′-diethylamino)eth-1-yl]amino, 7-methyl-2,7-diazaspiro[4.4]non-2-yl, 5-methyl-2,5-diazabicyclo[2.2.1]heptyl-2-yl, 4-methyl-1,4-diazepan-1-yl, piperidinyl, 4-carboxy-piperidinyl, 3-carboxypiperidinyl, 4-hydroxypiperidinyl, 4-(2-hydroxyeth-1-yl)piperidin-1-yl, 4-(N,N-dimethylamino)-piperidin-1-yl, 3-(N,N-dimethylamino) -methylpiperidin-1-yl, 2-(2-(N,N-dimethylamino)-eth-1-yl)piperidin-1-yl, 4-(4-methyl-4H-1,2,4-triazol-3-yl)piperidin-1-yl, 4-pyrrolidinyl-piperidinyl, 3-pyrrolidinyl-piperidinyl, 4-(N,N-diethylamino)-piperidin-1-yl, 4-(azetidin-1-yl)-piperidin-1-yl, 4-(piperidin-1-yl)-piperidin-1-yl, hexahydropyrrolo[1,2-a]pyrazin-2(1H)-yl, (2-(N,N-dimethylamino)-methyl)morpholino, 3,5-dimethylmorpholino, thiomorpholino, morpholino, pyrrolidinyl, 2-carboxy-pyrrolidin-1-yl, 2-(carboxy)-4-hydroxy-pyrrolidin-1-yl, 2-carboxamide-pyrrolidin-1-yl, 2-(N,N-dimethylaminocarbonyl) -pyrrolidin-1-yl, 3-(N′,N′-dimethylamino)-pyrrolidin-1-yl, 3-(N′,N′-diethylamino) -pyrrolidin-1-yl, 3-(pyridin-3-yl)-pyrrolidin-1-yl, 2-pyidin-4-ylpyrrolidin-1-y, piperazin-1-yl, 4-methylpiperazinyl, 4-(carboxymethyl)-piperazin-1-yl, 4-(2-hydroxyeth-1-yl) piperazin-1-yl, 4-(isopropyl)piperazin-1-yl, 4-(2-methoxyeth-1-yl)piperazin-1-yl, 4-(ethyl)piperazin-1-yl, 4-(N′,N′-dimethylaminoacetyl)-piperazin-1-yl, and 4-(6-methoxypyridin-2-yl) piperazin-1-yl.

44. A compound of claim 28 wherein t is 0.

45. A compound of claim 28 wherein t is 1 and X is amino, nitro, methyl or halo.

46. A compound of claim 28 wherein D is CH and E is S.

47. A compound selected from Table 1 or a tautomer, pharmaceutically acceptable salt, or partial salt thereof.

48. A pharmaceutical composition comprising a pharmaceutically acceptable carrier and a therapeutically effective amount of a compound of any one of claims 1, 28, or 47.

49. A method for treating or preventing a viral infection in a mammal mediated at least in part by a virus in the Flaviviridae family of viruses, comprising administering to said mammal a composition of claim 48.

50. The method of claim 49 wherein the viral infection is mediated by hepatitis C virus.

51. The method of claim 49 in combination with a therapeutically effective amount of one or more agents active against hepatitis C virus.

52. The method of claim 51 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.
